(19) United States
(12) Patent Application Publication
(10) Pub. No.: US 2023/0265444 A1
(43) Pub. Date: Aug. 24, 2023

(54) GENE-EDITING IN CANNABIS PLANT

(71) Applicant: CANOPY GROWTH CORPORATION, Smiths Falls (CA)

(72) Inventor: Daniel Kevin FOWLER, Arvada, CO (US)

(21) Appl. No.: 18/174,103

(22) Filed: Feb. 24, 2023

Related U.S. Application Data
(60) Provisional application No. 63/313,673, filed on Feb. 24, 2022.
(60) Provisional application No. 63/315,739, filed on Mar. 2, 2022.
(60) Provisional application No. 63/320,926, filed on Mar. 17, 2022.
(60) Provisional application No. 63/397,983, filed on Aug. 15, 2022.

(51) Int. Cl. C12N 15/82; C12N 15/11

(52) U.S. Cl. CPC C12N 15/8202; C12N 15/11; C12N 2310/20

(57) ABSTRACT

The present technology is in the field of molecular biology and plant biology, particularly as it pertains to gene editing in plants of the genus Cannabis. More specifically, there are provided systems and methods for targeted DNA modifications and gene editing in Cannabis plant, including systems and methods for making such modifications using a CRISPR/Cas-system nuclease complex, kits comprising the CRISPR/Cas-system nuclease complex, and constructs encoding the same.

### CROSS-REFERENCE TO RELATED APPLICATIONS

This application claims the benefit of and priority to U.S. provisional patent application No. 63/313,673, filed on Feb. 25, 2022; to U.S. provisional patent application No. 63/315,739, filed on Mar. 2, 2022; to U.S. provisional patent application No. 63/320,926, filed on Mar. 17, 2022; and to U.S. provisional patent application No. 63/397,983, filed on Aug. 15, 2022; the content of all of which is herein incorporated in entirety by reference.

### ELECTRONIC FILE—SEQUENCE LISTING

This application hereby incorporates by reference the material of the electronic Sequence Listing filed concurrently herewith. The material in the electronic Sequence Listing is submitted as an XML document (.xml) file entitled “GENE-EDITING IN CANNABIS PLANT.xml” created on Feb. 22, 2023, which has a file size of 156,863 bytes, and is herein incorporated by reference in its entirety.

### FIELD OF TECHNOLOGY

The present technology is in the field of molecular biology and plant biology. More specifically, the technology relates to targeted DNA modifications and gene editing in Cannabis plant, including systems and methods for making such modifications using a CRISPR/Cas-system nuclease complex, kits comprising the CRISPR/Cas-system nuclease complex, and constructs encoding the same.

### BACKGROUND

Genetic mutants are critical for the study of gene functions in plants and for genetic improvement of crops. In the past decades, characterization of natural mutants has revealed many important biological mechanisms which are studied to understand plant function, fight disease, increase yields, and optimize desirable properties. Many studies have used physical, chemical, or biological (e.g., T-DNA/transposon insertion) mutagenesis to identify mutants and construct mutant libraries corresponding to tens of thousands of genes in model plants, such as Arabidopsis (Kuromori et al., 2006) and rice (Wu et al., 2003; Xie and Yang, 2013). However, random mutagenesis is imprecise and can produce many undesirable mutations and rearrangements. Further, large-scale screening of mutants can be tedious and costly. Plant research and crop genetic improvement require new technologies for targeted mutagenesis and precise editing of genes, and regulation of gene expression. Methods of genome editing that allow addition, deletion, or substitution of nucleotides in predefined genomic loci are greatly needed.

The emergence of programmable sequence-specific nucleases (SSNs) provided a breakthrough in genome manipulation. SSNs can induce double-stranded breaks (DSBs) in specific chromosomal sites. The resulting DSBs can be repaired by the error-prone non-homologous end joining (NHEJ) pathway, often producing nucleotide insertions, deletions, and substitutions. Another independent pathway, homology-directed repair (HDR), also can repair the DSBs if homologous donor templates are present at the time of DSB formation (Symington and Gautier, 2011). The zinc finger nucleases (ZFNs) were the first generation of SSNs and were used to edit plant genomes (Lloyd et al., 2005; Zhang and Voytas, 2011; Kumar et al., 2015). However, ZFN constructs are difficult to manipulate and costly, which greatly hinders their application in various organisms including plants. Later, the transcription activator-like effector nucleases (TALENs) adapted from Xanthomonas bacteria were developed as a more promising tool and applied firstly in plants. However, TALENs require construction of complicated tandem repeat domains in the TAL proteins.

Recently, the clustered regularly interspaced short palindromic repeat (CRISPR)-associated protein9 (Cas9)-based genome editing tool, CRISPR/Cas9, has been adapted from the type II CRISPR adaptive immunity system in the bacterium Streptococcus pyogenes (Jinek et al., 2012), and applied to genome editing in many organisms including plants (Doudna and Charpentier, 2014). CRISPR/Cas9 can induce efficiently targeted mutations based on base-pairing of the engineered single-guide RNAs (sgRNAs) to the target DNA sites (Jinek et al., 2012), and is thus much easier to manipulate than ZFNs and TALENs.

CRISPR/Cas9 technology involves three major components: the Cas9 protein, single guide RNA (sgRNA), and target sites directly upstream of a protospacer adjacent motif (PAM; this is NGG for SpCas9 from Streptococcus pyogenes (Jinek et al., 2012; Doudna and Charpentier, 2014). The artificial sgRNA is engineered by linking the DNA sequences that express the protospacer-containing CRISPR RNA (crRNA) and the trans-activating crRNA (tracrRNA); the 5′ terminal part of the sgRNA contains the target sequence (not including the PAM) for its pairing to the target site (Jinek et al., 2012; Cong et al., 2013). The sgRNA accurately mimics the original crRNA-tracrRNA duplex and greatly simplifies manipulation with the CRISPR/Cas9 system. The Cas9 protein and the sgRNA form a Cas9 nuclease complex (Jinek et al., 2012; Jiang et al., 2015). Crucially, the specific recognition of the Cas9/sgRNA nuclease complex to target sites can be designed simply by introducing the target sequences into the sgRNA (Jinek et al., 2012). The Cas9/sgRNA complex searches for a genomic target DNA sequence adjacent to a PAM, which is required for effective target recognition (Sternberg et al., 2014). Once the target site is located, Cas9 melts the target sequence so that the sgRNA can pair with the target complementary strand. The Cas9 nuclease then cuts both strands of the target DNA three bases upstream of the PAM, producing a blunt-ended DSB. This DSB can be repaired either by NHEJ, to produce mutations (insertions, deletions, substitutions) in the targeted site, or by HDR in the presence of a homologous donor DNA template for precise gene editing (e.g., to create specific mutations, insert new elements, or for knockin gene replacement). Multiple engineered sgRNAs with different target sequences can be used at the same time to direct Cas9 to their respective target sites, allowing for multiplex editing in a cell (Cong et al., 2013).

CRISPR/Cas9 systems have been developed for gene editing of plant genomes, in various plants such as Arabidopsis thaliana, Zea mays, rice and tobacco (for review, see Ma et al., 2016; Belhaj et al., 2015; Kumar and Jain, 2015; Osakabe and Osakabe, 2015; Shan and Gao, 2015; Bortesi and Fischer, 2015). Such systems have been used, for example, for gene knockout, gene knockin, genomic deletion, disruption of cis-regulatory elements, and suppression of virus infection. In addition, various other Cas enzymes from different bacteria have been successfully utilized as an alternative genome editing system in plants, including for example the Staphylococcus aureus Cas9 (SaCas9) (Kaya et al., 2016) and Francisella novicida Cas12a (Cpf1, FnCas12a) (Wu et al., 2020). However, due to the relatively low efficiency of the HDR pathway in plants and inefficient delivery of homologous donor sequences into transfected plant cells (Puchta and Fauser, 2014), genome editing in plants remains difficult and of low efficiency. Moreover, such systems have only been developed successfully for a limited number of plant species. CRISPR/Cas genome editing has not been achieved in many plant species, including Cannabis.

Cannabis sativa L. is a diploid (2n=20) annual species belonging to the genus Cannabis in the Cannabaceae family. Cannabis sativa is commonly known as cannabis, hemp, Indian hemp, marihuana, and marijuana. Cannabis has been cultivated throughout the centuries as a source of fiber and food, and for its therapeutic and recreational properties (Farnsworth, N. R., 1969). The pharmaceutical and recreational properties of this unique plant are associated with a unique class of terpenophenolic compounds known as cannabinoids. Cannabinoids interact with receptors of human and animal endocannabinoid systems and can lead to a plethora of potential medical and therapeutic effects (Di Marzo & Piscitelli, 2015). Cannabinoids are produced and stored in the glandular trichomes present on the surfaces of female inflorescences. Over 500 phytocannabinoids and non-cannabinoid constituents have been identified and/or isolated from C. sativa L, including the well-known psychoactive compound Δ9-tetrahydro-cannabinol (Δ9-THC) and non-psychoactive compounds such as cannabidiol (CBD) (an isomer of THC), cannabichromene (CBC) and cannabigerol (CBG) (ElSohly et al., 2017).

As the legal cannabis market size continues to grow, a consistent supply of high-quality cannabis products remains a priority. Many factors affect the yield, quality, and desirability of cannabis products. There is a need in the art for systems and methods that allow targeted genome modification of Cannabis plant cells.

### SUMMARY

The present technology is based on the discovery that the CRISPR/Cas system can be used for Cannabis plant genome engineering. The CRISPR/Cas system provides a relatively simple, effective tool for generating modifications in genomic DNA at selected sites. CRISPR/Cas systems can be used to create targeted DSBs or single-strand breaks, and can be used for, without limitation, targeted mutagenesis, gene targeting, gene replacement, targeted deletions, targeted inversions, targeted translocations, targeted insertions, and multiplexed genome modification through multiple DSBs in a single cell directed by co-expression of multiple targeting guide RNAs. This technology can be used to, for example, accelerate the rate of functional genetic studies in Cannabis plants, and to engineer Cannabis plants with improved characteristics, including without limitation enhanced Cannabinoid content, increased resistance to disease and stress, and heightened production of commercially valuable strains and compounds.

According to various aspects, the present technology relates to CRISPR/nuclease systems and methods for modifying the genomic material in cells from a Cannabis plant. In certain embodiments of the present technology, the CRISPR/nuclease systems and methods are CRISPR/Cas systems and methods.

According to various aspects, the present technology relates to systems and methods for modifying the genomic material in a Cannabis plant cell, comprising: (a) introducing into the Cannabis plant cell a nucleic acid molecule, wherein the nucleic acid molecule comprises a guide RNA or a sequence encoding the guide RNA, wherein the guide RNA binds to a target sequence in the genome of the Cannabis plant cell; and (b) introducing into the Cannabis plant cell a CRISPR endonuclease molecule or a nucleic acid molecule (DNA or RNA) encoding the CRISPR endonuclease molecule, wherein the CRISPR endonuclease molecule induces a double stranded break (DSB) at or near the sequence to which the guide RNA sequence is targeted.

In some embodiments of the present technology, step (a) comprises delivering to the Cannabis plant cell a DNA encoding the guide RNA, and step (b) comprises delivering to the Cannabis plant cell a nucleic acid molecule (DNA or RNA) comprising the sequence encoding the CRISPR endonuclease molecule.

In some embodiments of the present technology, step (a) comprises delivering to the Cannabis plant cell a DNA encoding the guide RNA, and step (b) comprises delivering to the Cannabis plant cell a DNA comprising the sequence encoding the CRISPR endonuclease molecule.

In some embodiments of the present technology, steps (a) and (b) are combined into one step, the nucleic acid molecules encoding the guide RNA and the CRISPR endonuclease being linked on the same expression vector, which is introduced into the Cannabis plant cell.

In some embodiments of the present technology, nucleic acid molecules are introduced or delivered to the Cannabis plant cell via transformation, for example and without limitation, via a DNA virus (e.g., a geminivirus), an RNA virus (e.g., a tobravirus), via protoplasts, via transfer DNA (T-DNA) delivery (e.g., Agrobacterium or Ensifer, e.g., Agrobacterium tumefaciens), or via particle bombardment (i.e., biolistic transformation). In a particular embodiment, nucleic acid molecules (DNA or RNA) are delivered to the Cannabis plant cell via biolistic transformation. In another embodiment, nucleic acid molecules (DNA or RNA) are delivered to the Cannabis plant cell by transforming the cell with an Agrobacterium, e.g., Agrobacterium tumefaciens, carrying the nucleic acid molecule.

Alternative embodiments in which only one of the nucleic acid molecule encoding the guide RNA and the nucleic acid molecule encoding the CRISPR endonuclease are introduced into the Cannabis plant cell are also possible. Generally, in such embodiments, the Cannabis plant cell is a transgenic cell engineered to express one of the guide RNA and the CRISPR endonuclease, and the other is introduced into the cell via transformation.

In some embodiments of the present technology, the nucleic acid molecule is a DNA molecule. In some embodiments, the nucleic acid molecule is an RNA molecule.

In some embodiments of the present technology, DNA sequences encoding the guide RNA and/or the CRISPR endonuclease are each linked operably to a promoter that drives expression of the guide RNA and/or the CRISPR endonuclease in the cell. The promoter is not particularly limited and any promoter suitable for expression in the Cannabis plant cell may be used. For example, the promoter may be constitutive or inducible, and may or may not be tissue specific, cell-type specific, or development-specific. Non-limiting examples of promoters for use in accordance with the methods of the present technology include the 35S promoter, the AtU6 promoter, and the AtU3 promoter.

The DNA sequences encoding the guide RNA and the CRISPR endonuclease may be present on the same vector, or on different vectors. In some embodiments of the present technology, the DNA sequences encoding the guide RNA and the CRISPR endonuclease are present on the same vector.

In some embodiments of the present technology, vectors encoding the guide RNA and the CRISPR endonuclease may further comprise a selectable marker, such as, for example and without limitation, Hygromycin resistance. Other selectable markers that may be useful in the present technology include, but are not limited to, ampicillin resistance, kanamycin resistance, geneticin resistance, triclosan resistance, and URA3/5-fluoroorotic acid (5FOA) marker. In some embodiments of the present technology, vectors encoding the guide RNA and the CRISPR endonuclease may further comprise additional sequences such as, without limitation: a transcription terminator (e.g., the heat shock protein (HSP) terminator, the nopaline synthase (NOS) terminator, and the like); sequences allowing genomic integration (e.g., left border (LB) and right border (RB) sequences); and other sequences as may be desirable for function, stability, or transformation of the vector.

The CRISPR endonuclease molecule is not particularly limited. Any nuclease suitable for use as part of a Clustered Regularly Interspaced Short Palindromic Repeats (CRISPR) system may be used. For example and without limitation, the CRISPR endonuclease may be a Cas9, Cas 12, Cpf1, Csm1, CasX or CasY nuclease.

In some embodiments of the present technology, the CRISPR endonuclease molecule is a Cas enzyme. For example and without limitation, the Cas enzyme for use in the present technology may be Cas9, a Cas9 variant, Cas12, a Cas12 variant, CasX, or CasY. Different Cas enzymes, including different Cas9 or Cas12 variants, recognize different PAM sequences and may therefore be chosen based on the sequence of the target gene. For example, Cas12a recognizes a T-rich protospacer-adjacent motif (PAM), while Cas9 recognizes a G-rich PAM. Additional non-limiting examples of Cas enzymes for use in the present technology include Cas3, Cas4, Cas5, Cas5e (or CasD), Cas6, Cas6e, Cas6f, Cas7, Cas8a1, Cas8a2, Cas8b, Cas8c, Cas9, Cas10, CastlOd, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csxl7, Csx14, CsxlO, Csx16, CsaX, Csx3, Csz1, Csx15, Csf1, Csf2, Csf3, Csf4, Cu1966, bacteriophage Cas such as CasΦ (Cas-phi), and any combination thereof.

In some embodiments of the present technology, the CRISPR endonuclease molecule is a Cas9 enzyme. In an embodiment, the Cas9 enzyme is SpCas9. In an embodiment, the Cas12 enzyme is Cas12a (also known as Cpf1), e.g., AsCas12a, FnCas12a, or LbCas12a. In some embodiments, the CRISPR endonuclease molecule is linked to a nuclear localization signal (NLS) for transport into the nucleus. Any suitable nuclear localization signal may be used. For example and without limitation, the Simian virus 40 (SV40) T antigen nuclear localization sequence may be used. In an embodiment, the CRISPR endonuclease is SpCas9-NLS, wherein the NLS refers to the Simian virus 40 (SV40) T antigen nuclear localization sequence. In an embodiment, the CRISPR endonuclease is LbCas12a-NLS, wherein the NLS refers to the Simian virus 40 (SV40) T antigen nuclear localization sequence.

In some embodiments, the nucleic acid molecule encoding the CRISPR endonuclease molecule is plant codon optimized.

Different CRISPR endonuclease molecules also have different requirements for guide RNA. For example, Cas12 requires only CRISPR-derived RNA (crRNAs), whereas Cas9 requires a single guide RNA (sgRNA) derived from the fusion of CRISPR RNA (crRNA) and trans-activating crRNA (tracrRNA) (Burgess, 2013; Fonfara et al., 2016). The guide RNA is therefore chosen based on the CRISPR endonuclease molecule being used and the sequence of the target site in the genome. In some embodiments, the guide RNA is a single guide RNA (sgRNA). In some embodiments, the guide RNA is crRNA or a plurality of crRNAs. For example, multiple crRNAs can be expressed as a single transcript to generate functional individual crRNAs after processing through Cas12a nuclease. In another example, multiple guide RNAs, e.g. multiple sgRNAs, can be expressed on a single vector (as a single transcript or as separate transcripts) to allow multiplex editing of multiple target genes through Cas9 nuclease. It should be understood that the type and number of guide RNAs are not particularly limited and will be chosen based on the CRISPR endonuclease molecule being used and the desired gene-editing activity in the genome.

In some embodiments of the present technology, the CRISPR endonuclease molecule is SpCas9-NLS and the guide RNA is an sgRNA. In some such embodiments, a plurality of sgRNAs are used. The plurality of sgRNAs may target the same genomic site, e.g., to increase efficiency of gene-editing, or may target different genomic sites, e.g., to allow multiplex gene editing.

In some embodiments of the present technology, the CRISPR endonuclease molecule is Cas12a-NLS and the guide RNA is a crRNA. In some such embodiments, a plurality of crRNAs are used. The plurality of crRNAs may target the same genomic site, e.g., to increase efficiency of gene-editing, or may target different genomic sites, e.g., to allow multiplex gene editing.

In some embodiments of the present technology, the guide RNA comprises or consists of the sequence set forth in any one of SEQ ID NOs: 1-42, or has at least, greater than or about 75%, 80%, 85%, 90%, 95%, 96%, 97%, 98% or 99% sequence identity thereto.

In some embodiments of the present technology, the guide RNA has the sequence set forth in any one of SEQ ID NOs: 1-42.

In some embodiments of the present technology, the guide RNA binds and/or targets one or more of the following genes: CBDAS, CBCAS, R3-MYB1, R3-MYB2, R3-MYB3, R3-MYB4, and eIF4e.

The target gene or sequence to be modified using the systems and methods of the present technology is not particularly limited. Any desired genomic sequence may be targeted, as long as the requirements for cleavage by the CRISPR endonuclease are met (e.g., an appropriate PAM sequence must be present). For example and without limitation, the target gene may be CBDAS, CBCAS, R3-MYB1, R3-MYB2, R3-MYB3, R3-MYB4, eIF4e, or a combination thereof.

The Cannabis plant cell-type is also not particularly limited. In some embodiments of the present technology, the Cannabis plant cell is a somatic cell, e.g., a callus cell or a cell from a somatic embryo. In some embodiments, the somatic cell is immortalized. In some embodiments, the embryonic somatic cell is immortalized. For example and without limitation, the embryonic somatic cell may be an immortalized cell line. In some embodiments, the callus cell has been produced from young leaf tissue from Cannabis plantlets (i.e., a leaf-derived callus) and cultured. Calli may form a compact callus which is capable of forming somatic embryos. In some embodiments therefore, the Cannabis plant cell is a somatic embryonic cell in a compact callus, the compact callus being capable of forming somatic embryos. In other embodiments, the Cannabis plant cell is a somatic embryonic cell formed out of a non-compact callus, e.g., a female derived SE formed out of a non-compact callus.

In some embodiments of the present technology, gene-editing methods further comprise a step of cultivating cells into which one or more exogenous nucleic acid has been introduced (e.g., transformed cells) under conditions allowing gene-editing of the target gene in the genome of the cells. For example, cells may be cultivated under conditions allowing expression and/or activity of the introduced nucleic acid molecule(s). Conditions may allow transient expression and/or activity of the introduced nucleic acid molecule(s), or may allow genomic integration of an introduced nucleic acid molecule or a portion thereof. Such conditions for cultivation and/or culturing are known to the person skilled in the art.

In some embodiments of the present technology, gene-editing methods further comprise a step of selecting cells which have been gene-edited. For example and without limitation, a genomic region comprising the target gene or site may be amplified, e.g., using PCR, and then sequenced to determine if the target gene has been edited. In some embodiments, genomic DNA is first extracted from the cells. Any suitable method for selecting cells which have been gene-edited may be used.

The type of modification which may be made in the genome of the cells is not particularly limited. In some embodiments of the present technology, the modification of the genome comprises one or more nucleotide-editing event(s), e.g., insertion, deletion, or replacement of one or more nucleotides. In some embodiments of the present technology, the modification of the genome is selected from: i) a replacement of at least one nucleotide; ii) a deletion of at least one nucleotide; iii) an insertion of at least one nucleotide; and iv) any combination of i)-iii). In some such embodiments, the modification results from the CRISPR endonuclease cleaving the target site to form a double stranded break (DSB) which is repaired by NHEJ to produce mutations (insertions, deletions, substitutions).

In some embodiments of the present technology, the modification of the genome comprises a specific desired mutation, insertion of new elements into the genome or knockin gene replacement. In some such embodiments, the gene-editing methods of the technology further comprise introducing a homologous donor DNA template into the cells, such that the CRISPR endonuclease cleavage site is repaired by homology-directed repair (HDR), thereby allowing precise gene editing.

According to various aspects, the present technology relates to systems and methods for genetically editing a target gene in the genome of cells from a Cannabis plant, the method comprising: (a) introducing one or more exogenous nucleic acid having gene-editing activity into the cells; (b) optionally, cultivating the cells under conditions allowing gene-editing of the target gene in the genome of said cells; and (c) optionally, selecting cells which have been gene-edited by the gene-editing activity of the one or more exogenous nucleic acid molecule.

In some embodiments of the present technology, there is provided a method for genetically editing a target gene in the genome of cells from a Cannabis plant, the method comprising: (a) transforming the cells with one or more exogenous nucleic acid molecule having gene-editing activity; (b) optionally, cultivating the transformed cells under conditions allowing gene-editing of the target gene in the genome of said cells; and (c) optionally, selecting transformed cells which have been gene-edited by the gene-editing activity of the one or more exogenous nucleic acid molecule.

In some embodiments of the present technology, the present disclosure provides a method for genetically editing a Cannabis plant cell, the method comprising: transforming the Cannabis plant cell with an Agrobacterium cell carrying one or more exogenous nucleic acid molecule, and selecting transformed Cannabis plant cells which are gene-edited by the one or more exogenous nucleic acid molecule. In some embodiments, the one or more exogenous nucleic acid molecule is a DNA. In some embodiments, the one or more exogenous nucleic acid molecule is an RNA. In some embodiments, the one or more exogenous nucleic acid molecule comprises one or more of: a DNA encoding a single guide RNA (sgRNA), or an sgRNA; and a DNA encoding a Cas enzyme, or an RNA encoding a Cas enzyme. In some embodiments, the one or more exogenous nucleic molecule comprises a DNA encoding an sgRNA. In some embodiments, the one or more exogenous nucleic acid molecule comprises a DNA encoding a Cas9 enzyme. In further embodiments, the one or more exogenous nucleic acid molecule further comprises a homologous donor DNA template for directing specific desired modifications of the target gene.

In some embodiments of methods of the present disclosure, the methods further comprise a step of generating a female Cannabis plant from the genetically modified Cannabis plant cell obtained using the methods described herein, wherein the genetically modified Cannabis plant cell is a male founder cell, e.g., from a male founder somatic embryogenic cell line.

According to various aspects, the present disclosure provides genetically modified Cannabis plant cells obtained or obtainable by the methods described herein. The genetically modified plant cells may be, for example and without limitation, somatic cells, callus cells, somatic cells of a Cannabis somatic embryo, e.g., somatic cells of a compact callus capable of forming somatic embryos. In some embodiments, the genetically modified plant cells are male founder somatic cells, e.g., capable of generating a female Cannabis plant. In some embodiments, the genetically modified plant cells are immortalized cells.

According to various aspects, the present disclosure provides genetically modified Cannabis plants, plant parts, tissues, or cell lines which comprise the genetically modified Cannabis plant cells described herein.

According to various aspects, the present disclosure provides genetically modified Cannabis plants, plant parts, tissues, or cell lines which are generated from the genetically modified Cannabis plant cells described herein.

In some embodiments of the present technology, the genetically modified Cannabis plant, plant part, tissue, or cell line comprises one or more modification, compared to wild type Cannabis plant, in one or more of the following target genes: CBDAS, CBCAS, R3-MYB1, R3-MYB2, R3-MYB3, R3-MYB4, and eIF4e.

In some embodiments of the present technology, the genetically modified Cannabis plant, plant part, tissue, or cell line is a female Cannabis plant that has been generated from the genetically modified Cannabis plant cell described herein. In some such embodiments, the genetically modified Cannabis plant cell is a male founder cell, e.g., from a male founder somatic embryogenic cell line.

In some embodiments of the present technology, the genetically modified Cannabis plant, plant part, tissue, or cell line has a low THC and high CBD chemotype. In some embodiments, the genetically modified Cannabis plant, plant part, tissue, or cell line has a tetrahydrocannabinol (THC) content of between about 0.05% and about 0.25% by weight, and/or a cannabidiol (CBD) content of between about 0.01% and about 10% by weight.

According to various aspects, the present disclosure provides a vector for genetically editing a target gene in the genome of cells from a Cannabis plant, the vector comprising a nucleic acid encoding a CRISPR endonuclease, operably linked to a first heterologous promoter; and a nucleic acid encoding a guide RNA, operably linked to a second heterologous promoter. Such vectors are also referred to as “gene-editing vectors”. The first heterologous promoter and the second heterologous promoter may be the same promoter or may be different promoters. The promoter is not particularly limited, and any suitable promoter may be used. For example and without limitation, the first heterologous promoter and the second heterologous promoter may be, independently, a constitutive promoter, an inducible promoter, a tissue-specific promoter, or a development-specific promoter. In some embodiments, the first heterologous promoter and/or the second heterologous promoter is a 35S promoter, an AtU6 promoter, or an AtU3 promoter. In some embodiments, the vector further encodes a selectable marker, e.g., Hygromycin resistance. In some embodiments, the vector further comprises sequences for integrating into the genome and is capable of integrating into the genome.

In some embodiments, the vector encodes multiple guide RNAs. The multiple guide RNAs may target the same or different target genes in the genome.

In some embodiments of the present technology, the vector comprises or consists of the sequence set forth in any one of SEQ ID NOs: 43-58, or has at least, greater than or about 75%, 80%, 85%, 90%, 95%, 96%, 97%, 98% or 99% sequence identity thereto.

According to various aspects, the present disclosure provides kits for genomic DNA modification in a Cannabis plant. In some embodiments, the disclosure provides kit for genetically editing a target gene in the genome of cells from a Cannabis plant, the kits comprising one or more nucleic acid molecule as described herein, and instructions for use thereof. In some embodiments, the kit comprises a vector as described herein. In some embodiments, the kit comprises one or more guide RNA (or nucleic acid encoding a guide RNA); a nucleic acid encoding a CRISPR endonuclease, e.g., a Cas9 enzyme, and/or instructions for use. A kit may also include a homologous DNA template for homology-directed repair (HDR). A kit may also include reagents, solvents, buffers, etc., required for carrying out the methods described herein.

According to various aspects, the present technology relates to transgenic Cannabis plants, plant parts, tissues, cells, and cell lines, comprising at least one mutated gene encoding a cannabinoid biosynthesis enzyme. In some embodiments, the cannabinoid biosynthesis enzyme is selected from the group consisting of CBDAS, CBCAS, R3-MYB1, R3-MYB2, R3-MYB3, R3-MYB4, eIF4e, and combinations thereof. In some such embodiments, the at least one mutated gene is obtained using methods described herein, e.g., by targeted genome modification using at least one guide RNA as described herein, for example and without limitation, a guide RNA having the sequence set forth in any one of SEQ ID NOs: 1-42. In an embodiment, there is provided a transgenic Cannabis plant, plant part, tissue, cell, or cell line, comprising a mutated gene encoding for CBDAS, a mutated gene encoding for CBCAS, a mutated gene encoding for R3-MYB1, a mutated gene encoding for R3-MYB2, a mutated gene encoding for R3-MYB3, a mutated gene encoding for R3-MYB4, and/or a mutated gene encoding for eIF4e. In an embodiment, there is provided a transgenic Cannabis plant, plant part, tissue, cell, or cell line, comprising a knockout mutation in at least one gene encoding a cannabinoid biosynthesis enzyme, e.g., CBDAS, CBCAS, R3-MYB1, R3-MYB2, R3-MYB3, R3-MYB4, eIF4e, or a combination thereof. In some embodiments, there are provided transgenic Cannabis plants, plant parts, tissues, cells, and cell lines comprising one or more vector of the present technology.

According to various aspects, the present technology relates to methods for producing at least one Cannabis somatic embryo, the methods comprising: (a) culturing a Cannabis explant in a first medium comprising an auxin to form roots; (b) culturing the roots in a second medium comprising a first cytokinin to induce formation of callus; and (c) culturing the callus in a third medium comprising a second cytokinin to produce the at least one Cannabis somatic embryo. In some embodiments, the Cannabis explant is obtained from shoots, leaves, stems, flowers, and/or roots. In some embodiments, the Cannabis explant is a hypocotyl, cotyledon, or a mixture thereof.

Non-limiting examples of auxins for use in methods described herein include indole-3-acetic acid (IAA), 4-chloroindole-3-acetic acid (4-Cl-IAA), phenylacetic acid (PAA), indole-3-butyric acid (IBA), indole-3-propionic acid (IPA), indole-3-pyrruvic acid (IPyA), indole-3-acetamide (IAM), indole-3-ethanol (IAEt), indole-3-acetaldehyde (IAAld), indole-3-acetonitrile (IAN), tryptophan (TRP), tryptamine (TRA), 1-naphthaleneacetic acid (NAA), 2,4-dichlorophenoxyacetic acid (2,4-D), 3,6-dichloro-2-methoxybenzoic acid (Dicamba), 4-amino-3,5,6-trichloropyridine-2-carboxylic acid (Picloram), and any combination thereof.

In some embodiments, the first cytokinin is an adenine-type cytokinin, a phenylurea-type cytokinin, or a combination thereof.

In some embodiments, the first cytokinin is Nisopentenyladenine (iP), N6-isopentenyladenosine (iPR), N6-isopentenyladenine-7-glucoside (iP7G), N6-isopentenyladenine-9-glucoside (iP9G), N6-isopentenyladenosine-5′-monophosphate (iPRMP), N6-isopentenyladenosine-5′-diphosphate (iPRDP), N6-isopentenyladenosine-5′-triphosphate (iPRTP), trans-zeatin (tZ), trans-zeatin glucoside (tZR), trans-zeatin-7-glucoside (tZ7G), trans-zeatin-9-glucoside (tZ9G), trans-zeatin O-glucoside (tZOG), trans-zeatin riboside O-glucoside (tZROG), trans-zeatin riboside-5′-monophosphate (tZRMP), trans-zeatin riboside-5′-diphosphate (tZRDP), trans-zeatin riboside-5′-triphosphate (tZRTP), cis-zeatin (cZ), cis-zeatin glucoside (cZR), cis-zeatin-9-glucoside (cZ9G), cis-zeatin O-glucoside (cZOG), cis-zeatin riboside O-glucoside (cZROG), cis-zeatin riboside-5′-monophosphate (cZRMP), cis-zeatin riboside-5′-diphosphate (cZRDP), cis-zeatin riboside-5′-triphosphate (cZRTP), N6-benzyladenine (BA), N6-benzyladenosine (BAR), N6-benzyladenine-3-glucoside (BA3G), N6-benzyladenine-7-glucoside (BA7G), N6-benzyladenine-9-glucoside (BA9G), N6-benzyladenosine-5′-monophosphate (BARMP), N6-benzyladenosine-5′-diphosphate (BARDP), N6-benzyladenosine-5′-triphosphate (BARTP), dihydrozeatin (DHZ), dihydrozeatin glucoside (DHZR), dihydrozeatin-9-glucoside (DHZ9G), dihydrozeatin O-glucoside (DHZOG), dihydrozeatin riboside O-glucoside (DHZROG), dihydrozeatin riboside-5′-monophosphate (DHZRMP), dihydrozeatin riboside-5′-diphosphate (DHZRDP), dihydrozeatin riboside-5′-triphosphate (DHZRTP), ortho-topolin (oT), ortho-topolin riboside (oTR), ortho-topolin-9-glucoside (oT9G), meta-topolin (mT), meta-topolin riboside (mTR), meta-topolin-9-glucoside (mT9G), para-topolin (pT), para-topolin riboside (pTR), kinetin (K), kinetin riboside (KR), kinetin-9-glucoside (K9G), diphenylurea (DPU), thidiazuron (TDZ), or any combination thereof.

In some embodiments, the second cytokinin is an adenine type cytokinin, a phenylurea-type cytokinin, or a combination thereof.

In some embodiments, the second cytokinin is Nisopentenyladenine (iP), N⁶-isopentenyladenosine (iPR), N⁶-isopentenyladenine-7-glucoside (iP7G), N⁶-isopentenyladenine-9-glucoside (iP9G), N⁶-isopentenyladenosine-5′-monophosphate (iPRMP), N⁶-isopentenyladenosine-5′-diphosphate (iPRDP), N⁶-isopentenyladenosine-5′-triphosphate (iPRTP), trans-zeatin (tZ), trans-zeatin glucoside (tZR), trans-zeatin-7-glucoside (tZ7G), trans-zeatin-9-glucoside (tZ9G), trans-zeatin O-glucoside (tZOG), trans-zeatin riboside O-glucoside (tZROG), trans-zeatin riboside-5′-monophosphate (tZRMP), trans-zeatin riboside-5′-diphosphate (tZRDP), trans-zeatin riboside-5′-triphosphate (tZRTP), cis-zeatin (cZ), cis-zeatin glucoside (cZR), cis-zeatin-9-glucoside (cZ9G), cis-zeatin O-glucoside (cZOG), cis-zeatin riboside O-glucoside (cZROG), cis-zeatin riboside-5′-monophosphate (cZRMP), cis-zeatin riboside-5′-diphosphate (cZRDP), cis-zeatin riboside-5′-triphosphate (cZRTP), N⁶-benzyladenine (BA), N⁶-benzyladenosine (BAR), N⁶-benzyladenine-3-glucoside (BA3G), N⁶-benzyladenine-7-glucoside (BA7G), N⁶-benzyladenine-9-glucoside (BA9G), N⁶-benzyladenosine-5′-monophosphate (BARMP), N⁶-benzyladenosine-5′-diphosphate (BARDP), N⁶-benzyladenosine-5′-triphosphate (BARTP), dihydrozeatin (DHZ), dihydrozeatin glucoside (DHZR), dihydrozeatin-9-glucoside (DHZ9G), dihydrozeatin O-glucoside (DHZOG), dihydrozeatin riboside O-glucoside (DHZROG), dihydrozeatin riboside-5′-monophosphate (DHZRMP), dihydrozeatin riboside-5′-diphosphate (DHZRDP), dihydrozeatin riboside-5′-triphosphate (DHZRTP), ortho-topolin (oT), ortho-topolin riboside (oTR), ortho-topolin-9-glucoside (oT9G), meta-topolin (mT), meta-topolin riboside (mTR), meta-topolin-9-glucoside (mT9G), para-topolin (pT), para-topolin riboside (pTR), kinetin (K), kinetin riboside (KR), kinetin-9-glucoside (K9G), diphenylurea (DPU), thidiazuron (TDZ), or any combination thereof.

Other aspects and features of the present disclosure will become apparent to those ordinarily skilled in the art upon review of the following description of specific embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 is a photograph of a gel showing the results of in vitro Cas9-RNP cleavage of CBDAS or CBCAS double-stranded DNA (dsDNA) with guide RNAs as indicated or control without guide RNA. Cleavage of the control size band produces two smaller bands of varying size depending on where Cas9 cuts in the dsDNA.

FIG. 2 is a photograph of a gel showing the results of in vitro Cas12a-RNP cleavage of CBDAS or CBCAS dsDNA with guide RNAs as indicated or control without guide RNA. Cleavage of the control size band produces two smaller bands of varying size depending on where Cas12a cuts in the dsDNA.

FIG. 3 is a photograph of a gel showing the results of in vitro Cas9-RNP cleavage of R3-MYB1 or MYB2 dsDNA with guide RNAs as indicated or control without guide RNA.

FIG. 4 is a photograph of a gel showing the results of in vitro Cas9-RNP cleavage of R3-MYB3 or MYB4 dsDNA with guide RNAs as indicated or control without guide RNA.

FIG. 5 is a photograph of a gel showing the results of in vitro Cas12a-RNP cleavage of R3-MYB1 or MYB2 dsDNA with guide RNAs as indicated or control without guide RNA.

FIG. 6 is a photograph of a gel showing the results of in vitro Cas9-RNP cleavage of eIF4e with guide RNAs as indicated or control without guide RNA.

FIG. 7 shows Sanger sequencing results for wild type and Cas9 gene-edited CBDAS gene. The Cas9 cleavage site is indicated with an arrow, and the inserted nucleotide is indicated with an asterisk.

FIG. 8 shows Sanger sequencing results for wild type and Cas9 gene-edited CBCAS gene. The Cas9 cleavage site is indicated with an arrow, and the deleted nucleotide is indicated with an asterisk.

FIG. 9 shows Sanger sequencing results for wild type and Cas9 gene-edited R3-MYB1, R3-MYB2a, and R3-MYB2b genes. Cleavage and editing sites are indicated with arrows.

FIG. 10 is a photograph of globular embryos grown from a founding male somatic embryogenic (SE) line.

FIG. 11 is a photograph of heart and torpedo embryos grown from a founding male somatic embryogenic (SE) line.

FIG. 12 is a photograph of mature embryos grown from a founding male somatic embryogenic (SE) line SE1-11G that was edited at the CBDAS gene. White arrows indicate shoot and root poles on a somatic embryo, and the red arrows indicate additional somatic embryos.

FIG. 13 is a photograph of a regenerating plant in tissue culture grown from a somatic embryo induced from CBDAS-edited male founder SE line SE1-11G.

FIG. 14 shows Sanger sequencing results for a region of CBDAS amplified from genomic DNA extracted from wild type male SE founder line (left) and from regenerating plantlet grown from SE1-11G cell line edited at the CBDAS gene (right). Gene editing is indicated both by overlaid peaks and the asterisk shows a nucleotide insertion in the CBDAS gene. The Cas9 cleavage site is indicated with an arrow.

FIG. 15 is a photograph of shoots developed from the wild type male founder SE line in a bioreactor.

FIG. 16 is a photograph of a hardened male founder SE line plant.

FIG. 17 is a photograph of shoots developed from the female derived SE line SE2a in a bioreactor.

FIG. 18 is a photograph showing an image from bright field microscopy (left) and fluorescent microscopy (right) of GFP-positive SEs grown from the female derived SE line SE2a-1.

FIG. 19 is a schematic drawing of the pRI-Cas9-HygroR vector.

FIG. 20 shows alignment of the first 260 nucleotides of the CBDAS coding sequence determined by clonal sequence analysis for the edited plantlet 11G (shown in FIGS. 13-14) and the unedited wild type male SE founder line.

FIG. 21 shows alignment of CBDAS amino acid sequences determined by clonal sequence analysis for the edited plantlet 11G (shown in FIGS. 13-14) and the unedited wild type male SE founder line.

FIG. 22 are photographs of additional regenerating plants in tissue cultures grown from somatic embryos induced from CBDAS-edited male founder SE line SE1-11G.

FIG. 23 are photographs of hardened flowering plants with anthers of CBDAS-edited male founder SE line SE1-11G.

FIG. 24 are photographs of pollen-producing anthers of CBDAS-edited male founder SE line SE1-11G.

FIG. 25 are photographs of hardened derived female SE line SE2a plants.

FIG. 26 are photographs of hardened derived female SE line SE4c plants.

### DETAILED DESCRIPTION

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure in which variations and additions do not depart from the present technology. Hence, the following description is intended to illustrate some particular embodiments of the technology, and not to exhaustively specify all permutations, combinations and variations thereof.

### Definitions

Unless otherwise defined, all technical and scientific terms used herein have the same meaning as commonly understood by one of ordinary skill in the art to which this technology pertains.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.25, 1.5, 1.75, 2, 2.45, 2.75, 3, 3.80, 4, 4.32, and 5).

The term “about” is used herein explicitly or not. Every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. The term “or” as used herein should in general be construed non-exclusively. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

As used herein, the term “comprise” is used in its non-limiting sense to mean that items following the word are included, but items not specifically mentioned are not excluded.

As used herein, the term “Cannabis” refers to the genus of flowering plants in the family Cannabaceae regardless of species, subspecies, or subspecies variety classification. At present, there is no general consensus whether plants of genus Cannabis are comprised of a single or multiple species (McPartland & Guy, 2017). For example, some describe Cannabis plants as a single species, C. sativa L., with multiple subspecies (Small & Cronquist, 1976; McPartland & Small, 2020) while others classify Cannabis plants into multiple species, most commonly as C. sativa L. and C. indica Lam. and sometimes additionally as C. ruderalis Janisch. (Schultes et al., 1974), depending on multiple criteria including morphology, geographic origin, chemical content, and genetic measurements. Regardless, all plants of genus Cannabis can interbreed and produce fertile offspring (Small, 1972).

The term “strain” as used herein refers to different varieties of the plant genus Cannabis. For example, the term “strain” can refer to different pure or hybrid varieties of Cannabis plants. In some instances, the Cannabis strain of the present technology can by a hybrid of two strains. Different Cannabis strains often exhibit distinct chemical compositions with characteristic levels of cannabinoids and terpenes, as well as other components. Differing cannabinoid and terpene profiles associated with different Cannabis strains can be useful e.g. for the treatment of different diseases, or for treating different subjects with the same disease.

As used herein, the term “cannabinoid” refers to a chemical compound belonging to a class of secondary compounds commonly found in plants of genus Cannabis, but also encompasses synthetic and semi-synthetic cannabinoids and any enantiomers thereof. In an embodiment, the cannabinoid is a compound found in a plant, e.g., a plant of genus Cannabis, and is sometimes referred to as a phytocannabinoid. In one embodiment, the cannabinoid is a compound found in a mammal, sometimes called an endocannabinoid. In one embodiment, the cannabinoid is made in a laboratory setting, sometimes called a synthetic cannabinoid. In one embodiment, the cannabinoid is derived or obtained from a natural source (e.g. plant) but is subsequently modified or derivatized in one or more different ways in a laboratory setting, sometimes called a semi-synthetic cannabinoid.

Synthetic cannabinoids and semi-synthetic cannabinoids encompass a variety of distinct chemical classes, for example and without limitation: the classical cannabinoids structurally related to THC, the non-classical cannabinoids (cannabimimetics) including the aminoalkylindoles, 1,5 diarylpyrazoles, quinolines, and arylsulfonamides as well as eicosanoids related to endocannabinoids.

In another embodiment, a cannabinoid is one of a class of diverse chemical compounds that may act on cannabinoid receptors such as CB₁ and CB₂ in cells that alter neurotransmitter release in the brain.

In many cases, a cannabinoid can be identified because its chemical name will include the text string “*cannabi*”. However, there are a number of cannabinoids that do not use this nomenclature, such as for example those described herein.

As used herein, the expression “% by weight” is calculated based on dry weight of the total material.

Within the context of this disclosure, where reference is made to a particular cannabinoid, each of the acid and/or decarboxylated forms are contemplated as both single molecules and mixtures. In addition, salts of cannabinoids are also encompassed, such as salts of cannabinoid carboxylic acids. As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form). Further, in some embodiments of the present disclosure, the cannabinoid is a cannabinoid dimer. The cannabinoid may be a dimer of the same cannabinoid (e.g. THC-THC) or different cannabinoids. In an embodiment of the present disclosure, the cannabinoid may be a dimer of THC, including for example Cannabisol.

In an embodiment, a cannabinoid may occur in its free form, or in the form of a salt; an acid addition salt of an ester; an amide; an enantiomer; an isomer; a tautomer; a prodrug; a derivative of an active agent of the present technology; different isomeric forms (for example, enantiomers and diastereoisomers), both in pure form and in admixture, including racemic mixtures; enol forms.

As used herein, the expressions “nucleic acid,” “nucleic acid molecule,” “oligonucleotide,” and “polynucleotide” are each used herein to refer to a polymer of at least three nucleotides. In some embodiments, a nucleic acid comprises deoxyribonucleic acid (DNA). In some embodiments, a nucleic acid comprises ribonucleic acid (RNA). In some embodiments, a nucleic acid is single stranded. In some embodiments, a nucleic acid is double stranded. In some embodiments, a nucleic acid comprises both single and double stranded portions. Unless otherwise stated, the terms encompass nucleic acid-like structures with synthetic backbones, as well as amplification products. In some embodiments, nucleic acids of the present disclosure are expression vectors. In some embodiments, nucleic acids of the present disclosure are linear nucleic acids.

As used herein, the expression “nucleic acid having gene-editing activity” is used to refer to a nucleic acid that is, comprises, or encodes one or more component of a CRISPR/nuclease complex, i.e., a CRISPR endonuclease and/or a guide RNA. Nucleic acids having gene-editing activity are also referred to as “gene-editing nucleic acids”, e.g., “gene-editing vectors”, herein.

“Nuclease” and “endonuclease” are used interchangeably herein to mean an enzyme which possesses endonucleolytic catalytic activity for DNA cleavage. “CRISPR endonuclease” is used to refer to a site-specific nuclease enzyme used for CRISPR/nuclease gene editing and genome modification, e.g., Cas9, Cas12a, etc.

As used herein, the term “gene” refers to a part of the genome that codes for a product (e.g., an RNA product and/or a polypeptide product). Generally, a gene is a heritable sequence of DNA, i.e., a genomic sequence, with functional significance. The term “gene” can also be used to refer to, e.g., a cDNA and/or an mRNA encoded by a genomic sequence, as well as to that genomic sequence. A “gene sequence” is a sequence that includes at least a portion of a gene (e.g., all or part of a gene) and/or regulatory elements associated with a gene. In some embodiments, a gene includes coding sequence; in some embodiments, a gene includes non-coding sequence. In some particular embodiments, a gene may include both coding (e.g., exonic) and non-coding (e.g., intronic) sequences. In some embodiments, a gene may include one or more regulatory elements (e.g., a promoter) that, for example, may control or impact one or more aspects of gene expression (e.g., cell-type-specific expression, inducible expression, etc.).

As used herein, the expression “coding sequence” refers to a sequence of a nucleic acid or its complement, or a part thereof, that: i) can be transcribed to an mRNA sequence that can be translated to produce a polypeptide or a fragment thereof, or ii) an mRNA sequence that can be translated to produce a polypeptide or a fragment thereof. Coding sequences include exons in genomic DNA or immature primary RNA transcripts, which are joined together by the cell's biochemical machinery to provide a mature mRNA.

As used herein, the terms “mutation” and “modification” refers to a change introduced into a parental sequence, including, but not limited to, substitutions, insertions, deletions (including truncations). The consequences of a mutation include, but are not limited to, the creation of a new character, property, function, phenotype or trait not found in the protein encoded by the parental sequence, or the increase or reduction/elimination of an existing character, property, function, phenotype or trait not found in the protein encoded by the parental sequence.

The expression “degree or percentage of sequence identity” refers herein to the degree or percentage of sequence identity between two sequences after optimal alignment. Percentage of sequence identity (or degree of identity) is determined by comparing two aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact base-pairing. Thus, the stem may include one or more base mismatches. Alternatively, the base-pairing may be exact, i.e., not include any mismatches.

By “hybridizable” or “complementary” or “substantially complementary” it is meant that a nucleic acid (e.g., RNA) comprises a sequence of nucleotides that enables it to non-covalently bind, i.e., to form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C) (DNA, RNA). In addition, it is known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a guide RNA molecule is considered complementary to a uracil (U), and vice versa. As such, when a G/U base-pair can be made at a given nucleotide position in a protein-binding segment (dsRNA duplex) of a guide RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). It is well-known that the conditions of temperature and ionic strength determine the “stringency” of the hybridization.

Hybridization requires that two nucleic acids contain complementary sequences, although mismatches between bases are possible. The conditions appropriate for hybridization between two nucleic acids depend on the length of the nucleic acids and the degree of complementarity, variables well-known in the art. The greater the degree of complementarity between two nucleotide sequences, the greater the value of the melting temperature (Tm) for hybrids of nucleic acids having those sequences. For hybridizations between nucleic acids with short stretches of complementarity (e.g., complementarity over 35 or less, 30 or less, 25 or less, 22 or less, 20 or less, or 18 or less nucleotides) the position of mismatches becomes important (see Sambrook et al., supra, 11.7-11.8). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides. Furthermore, the skilled artisan will recognize that the temperature and wash solution salt concentration may be adjusted as necessary according to factors such as length of the region of complementarity and the degree of complementarity.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within a target nucleic acid sequence to which it is targeted. For example, an antisense nucleic acid in which 18 of 20 nucleotides of the antisense compound are complementary to a target region, and would therefore specifically hybridize, would represent 90 percent complementarity. In this example, the remaining non-complementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

As used herein, the term “isolated” refers to nucleic acids or polypeptides that have been separated from their native environment, including but not limited to virus, proteins, glycoproteins, peptide derivatives or fragments or polynucleotides. For example, the expression “isolated nucleic acid molecule” as used herein refers to a nucleic acid substantially free of cellular material or culture medium when produced by recombinant DNA techniques, or chemical precursors, or other chemicals when chemically synthesized. An isolated nucleic acid is also substantially free of sequences, which naturally flank the nucleic acid (i.e. sequences located at the 5′ and 3′ ends of the nucleic acid) from which the nucleic acid is derived.

Two nucleotide sequences or amino-acids are said to be “identical” if the sequence of nucleotide residues or amino-acids in the two sequences is the same when aligned for maximum correspondence as described below. Sequence comparisons between two (or more) peptides or polynucleotides are typically performed by comparing sequences of two optimally aligned sequences over a segment or “comparison window” to identify and compare local regions of sequence similarity. Optimal alignment of sequences for comparison may be conducted by the local homology algorithm of Smith and Waterman, Ad. App. Math 2: 482 (1981), by the homology alignment algorithm of Needleman and Wunsch, J. Mol. Biol. 48: 443 (1970), by the search for similarity method of Pearson and Lipman, Proc. Natl. Acad. Sci. (U.S.A.) 85: 2444 (1988), by computerized implementation of these algorithms (GAP, BESTFIT, FASTA, and TFASTA in the Wisconsin Genetics Software Package, Genetics Computer Group (GCG), 575 Science Dr., Madison, Wis.), or by visual inspection. Other alignment programs may also be used such as: “Multiple sequence alignment with hierarchical clustering”, F. CORPET, 1988, Nucl. Acids Res., 16 (22), 10881-10890.

As used herein, the expression “conservative substitutions” refers to a substitution made in an amino acid sequence of a polypeptide without disrupting the structure or function of the polypeptide. Conservative amino acid substitutions may be accomplished by substituting amino acids with similar hydrophobicity, polarity, and R-chain length for one another. Additionally, by comparing aligned sequences of homologous proteins from different species, conservative amino acid substitutions may be identified by locating amino acid residues that have been mutated between species without altering the basic functions of the encoded proteins. Amino acid substitutions that are conservative are typically as follows: i) hydrophilic: Alanine (Ala) (A), Proline (Pro) (P), Glycine (Gly) (G), Glutamic acid (Glu) (E), Aspartic acid (Asp) (D), Glutamine (Gln) (Q), Asparagine (Asn) (N), Serine (Ser) (S), Threonine (Thr) (T); ii) Sulphydryl: Cysteine (Cys) (C); iii) Aliphatic: Valine (Val) (V), Isoleucine (Ile) (I), Leucine (Leu) (L), Methionine (Met) (M); iv) Basic: Lysine (Lys) (K), Arginine (Arg) (R), Histidine (His) (H); and v) Aromatic: Phenylalanine (Phe) (F), Tyrosine (Tyr) (Y), Tryptophan (Trp) (W).

An “expression system” as used herein refers to reagents and components (e.g. in a kit) and/or solutions comprising said reagents and components for recombinant protein expression, wherein the expression system is cell free and includes optionally translation competent extracts of whole cells and/or other translation machinery reagents or components optionally in a solution, said reagents and components optionally including RNA polymerase, one or more regulatory protein factors, one or more transcription factors, ribosomes, and tRNA, optionally supplemented with cofactors and nucleotides, and the specific gene template of interest. Chemical based expression systems are also included, optionally using unnaturally occurring amino acids. In some instances, the expression systems of the present technology are in vitro expression systems.

The expressions “transformed with”, “transfected with”, “transformation” and “transfection” are intended to encompass introduction of nucleic acid (e.g., a construct) into a cell by one of many possible techniques known in the art.

The term “primer” as used herein, typically refers to oligonucleotides that hybridize in a sequence specific manner to a complementary nucleic acid molecule (e.g., a nucleic acid molecule comprising a target sequence). In some embodiments, a primer will comprise a region of nucleotide sequence that hybridizes to at least 8, e.g., at least 10, at least 15, at least 20, at least 25, or 20 to 60 nucleotides of a target nucleic acid (i.e., will hybridize to a sequence of the target nucleic acid). In general, a primer sequence is identified as being either “complementary” (i.e., complementary to the coding or sense strand (+)), or “reverse complementary” (i.e., complementary to the anti-sense strand (−)). In some embodiments, the term “primer” may refer to an oligonucleotide that acts as a point of initiation of a template-directed synthesis using methods such as PCR (polymerase chain reaction) under appropriate conditions (e.g., in the presence of four different nucleotide triphosphates and a polymerization agent, such as DNA polymerase in an appropriate buffer solution containing any necessary reagents and at suitable temperature(s)). Such a template directed synthesis is also called “primer extension.” For example, a primer pair may be designed to amplify a region of DNA using PCR. Such a pair will include a “forward primer” and a “reverse primer” that hybridize to complementary strands of a DNA molecule and that delimit a region to be synthesized and/or amplified.

As used herein, the expression “wild-type” refers to a typical or common form existing in nature; in some embodiments it is the most common form.

As used herein, “allele” generally refers to an alternative nucleic acid sequence at a particular locus; the length of an allele can be as small as 1 nucleotide base but is typically larger. For example, a first allele can occur on one chromosome, while a second allele occurs on a second homologous chromosome, e.g., as occurs for different chromosomes of a heterozygous individual, or between different homozygous or heterozygous individuals in a population. A “favorable allele” is the allele at a particular locus that confers, or contributes to, an agronomically desirable phenotype, or alternatively, is an allele that allows the identification of susceptible plants that can be removed from a breeding program or planting. A favorable allele of a marker is a marker allele that segregates with the favorable phenotype, or alternatively, segregates with susceptible plant phenotype, therefore providing the benefit of identifying disease prone plants. A favorable allelic form of a chromosome interval is a chromosome interval that includes a nucleotide sequence that contributes to superior agronomic performance at one or more genetic loci physically located on the chromosome interval.

“Plant” refers to a whole plant or any part thereof, such as a cell or tissue culture derived from a plant, comprising any of: whole plants, plant components or organs (e.g., leaves, stems, roots, etc,), plant tissues, seeds, plant cells, and/or progeny of the same. A plant cell is a biological cell of a plant, taken from a plant or derived through culture from a cell taken from a plant. As used herein, the expression “plant part” refers to any part of a plant including but not limited to the embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen, and the like. The two main parts of plants grown in some sort of media, such as soil or vermiculite, are often referred to as the “above-ground” part, also often referred to as the “shoots”, and the “below-ground” part, also often referred to as the “roots.” Plant part may also include certain extracts such as kief or hash which includes Cannabis trichomes or glands.

“Polymorphism” means the presence of one or more variations in a population. A polymorphism may manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation may comprise but is not limited to one or more nucleotide base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism, and a tag SNP. A genetic marker, a gene, a DNA-derived sequence, a RNA-derived sequence, a promoter, a 5′ untranslated region of a gene, a 3′ untranslated region of a gene, microRNA, siRNA, a tolerance locus, a satellite marker, a transgene, mRNA, ds mRNA, a transcriptional profile, and a methylation pattern may also comprise polymorphisms. In addition, the presence, absence, or variation in copy number of the preceding may comprise polymorphisms.

A “population of plants” or “plant population” means a set comprising any number, generally more than one, of individuals, objects, or data from which samples are taken for evaluation, e.g. estimating QTL effects. Most commonly, the terms relate to a breeding population of plants from which members are selected and crossed to produce progeny in a breeding program. A population of plants can include the progeny of a single breeding cross or a plurality of breeding crosses, and can be either actual plants or plant derived material, or in silica representations of the plants. The population members need not be identical to the population members selected for use in subsequent cycles of analyses or those ultimately selected to obtain final progeny plants. Often, a plant population is derived from a single biparental cross, but may also derive from two or more crosses between the same or different parents. Although a population of plants may comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population.

“Recombinant” in reference to a nucleic acid or polypeptide indicates that the material (e.g., a recombinant nucleic acid, gene, polynucleotide, polypeptide, etc.) has been altered by human intervention. “Recombinant,” as used herein, means that a particular nucleic acid (DNA or RNA) or vector is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms. Alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); and Cell and Tissue Culture: Laboratory Procedures in Biotechnology (Doyle & Griffiths, John Wiley & Sons 1998), the disclosures of which are incorporated herein by reference.

A “vector” or “expression vector” is a replicon, such as plasmid, phage, virus, or cosmid, to which another DNA segment, i.e., an “insert”, may be attached so as to bring about the replication of the attached segment in a cell. Vectors may also be referred to as “constructs” herein.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The nucleic acid(s) may or may not be operably linked to a promoter sequence and may or may not be operably linked to DNA regulatory sequences.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g., with reference to a DNA-binding or an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{d}) of less than 10⁻⁶ M, less than 10⁻⁷ M, less than 10⁻¹ M, less than 10⁻⁹ M, less than 10⁻¹⁰ M, less than 10⁻¹¹ M, less than 10⁻¹² M, less than 10⁻¹³ M, less than 10⁻⁴ M, or less than 10⁻¹⁵ M. “Affinity” refers to the strength of binding, increased binding affinity being correlated with a lower K_{d}. By “binding domain” it is meant a protein domain that is able to bind non-covalently to another molecule. A binding domain can bind to, for example, a DNA molecule (a DNA-binding protein), an RNA molecule (an RNA-binding protein) and/or a protein molecule (a protein-binding protein). In the case of a protein domain-binding protein, it can bind to itself (to form homodimers, homotrimers, etc.), and/or it can bind to one or more molecules of a different protein or proteins.

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g., tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

As used herein, a “promoter sequence” is a DNA regulatory region capable of binding RNA polymerase and initiating transcription of a downstream (3′ direction) coding or non-coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Various promoters, including inducible promoters, the 35S CaMV promoter, the AtU6 promoter, and the AtU3 promoter, etc., may be used to drive the various vectors of the present invention.

A promoter can be a constitutively active promoter (i.e., a promoter that is constitutively in an active/“ON” state), an inducible promoter (i.e., a promoter whose state, active/“ON” or inactive/“OFF”, is controlled by an external stimulus, e.g., the presence of a particular temperature, compound, or protein), a spatially restricted promoter (i.e., transcriptional control element, enhancer, etc.; e.g., a tissue specific promoter, a cell type specific promoter, etc.), and/or a temporally restricted promoter (i.e., the promoter is in the “ON” state or “OFF” state during specific stages of embryonic development or during specific stages of a biological process. Suitable promoters can be derived from viruses and can therefore be referred to as viral promoters, or they can be derived from any organism, including prokaryotic or eukaryotic organisms. Suitable promoters can be used to drive expression by any RNA polymerase (e.g., Pol I, Pol II, or Pol III). Exemplary promoters include, but are not limited to, the 35S CaMV promoter, the AtU6 promoter, and the AtU3 promoter.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., a guide RNA) or a coding sequence (e.g., a site-directed modifying polypeptide, or a Cas9 polypeptide) and/or regulate translation of an encoded polypeptide.

The term “naturally-occurring” or “unmodified” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including plants) that can be isolated from a source in nature and which has not been intentionally modified by a human in the laboratory is naturally occurring.

“Amplifying,” in the context of nucleic acid amplification, is any process whereby additional copies of a selected nucleic acid (or a transcribed form thereof) are produced. In some embodiments, an amplification based marker technology is used wherein a primer or amplification primer pair is admixed with genomic nucleic acid isolated from a cell, and wherein the primer or primer pair is complementary or partially complementary to a portion thereof and is capable of initiating DNA polymerization by a DNA polymerase using the genomic nucleic acid as a template. The primer or primer pair is extended in a DNA polymerization reaction having a DNA polymerase and a template genomic nucleic acid to generate at least one amplicon. As used herein, an “amplicon” is an amplified nucleic acid, e.g., a nucleic acid that is produced by amplifying a template nucleic acid by any available amplification method (e.g., PCR, LCR, transcription, or the like).

As used herein, a “genomic nucleic acid” is a nucleic acid that corresponds in sequence to a heritable nucleic acid in a cell. Common examples include nuclear genomic DNA and amplicons thereof. A genomic nucleic acid is, in some cases, different from a spliced RNA, or a corresponding cDNA, in that the spliced RNA or cDNA is processed, e.g., by the splicing machinery, to remove introns. Genomic nucleic acids optionally comprise non-transcribed (e.g., chromosome structural sequences, promoter regions, enhancer regions, etc.) and/or non-translated sequences (e.g., introns), whereas spliced RNA/cDNA typically do not have non-transcribed sequences or introns. “Genomic DNA” refers to the DNA of a genome of an organism including, but not limited to, the DNA of the genome of a bacterium, fungus, archea, plant or animal, including Cannabis plants.

A cell has been “transformed” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In some cases, the transforming DNA may be maintained on an episomal element such as a plasmid. With respect to eukaryotic cells, a “transgenic” cell is one in which the transforming DNA has become integrated into a chromosome so that it is inherited by daughter cells through chromosome replication. A “clone” is a population of cells derived from a single cell or common ancestor by mitosis. A “cell line” is a clone of a primary cell that is capable of stable growth in vitro for many generations.

A “host cell,” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a nucleic acid, and include the progeny of the original cell which has been transformed by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a bacterial host cell is a genetically modified bacterial host cell by virtue of introduction into a suitable bacterial host cell of an exogenous nucleic acid (e.g., a plasmid or recombinant expression vector) and a eukaryotic host cell is a genetically modified eukaryotic host cell (e.g., a somatic embryonic cell, a callus cell), by virtue of introduction into a suitable eukaryotic host cell of an exogenous nucleic acid.

A “target DNA” as used herein is a DNA polynucleotide that comprises a “target site” or “target sequence” or “target gene”. The terms “target site,” “target sequence,” “target DNA” and “target gene” are used interchangeably herein to refer to a nucleic acid sequence present in a target DNA to which a DNA-targeting segment of a guide RNA will bind, provided sufficient conditions for binding exist. For example, the target site (or target sequence) 5′-GAGCATATC-3′ within a target DNA is targeted by (or is bound by, or hybridizes with, or is complementary to) the RNA sequence 5′-GAUAUGCUC-3′. Suitable DNA/RNA binding conditions include physiological conditions normally present in a cell. Other suitable DNA/RNA binding conditions (e.g., conditions in a cell-free system) are known in the art; see, e.g., Sambrook, supra. The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “noncomplementary strand” or “non-complementary strand.” Guide RNA molecules comprise a sequence that binds, hybridizes to, or is complementary to a target sequence within the target DNA, thus targeting the bound nuclease to a specific location within the target DNA (the target sequence).

By “cleavage” is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, a complex comprising a guide RNA and a CRISPR endonuclease is used for targeted double-stranded DNA cleavage.

By “cleavage domain” or “active domain” or “nuclease domain” of a nuclease is meant the polypeptide sequence or domain within the nuclease which possesses the catalytic activity for DNA cleavage. A cleavage domain can be contained in a single polypeptide chain or cleavage activity can result from the association of two (or more) polypeptides. A single nuclease domain may consist of more than one isolated stretch of amino acids within a given polypeptide.

The terms “recombinant donor template” and “homologous DNA template” refer to a nucleic acid strand, e.g., a DNA strand, that is the recipient strand during homologous recombination strand invasion initiated after DNA damage or cleavage, in some cases, resulting from a double-stranded break. The donor polynucleotide serves as template material to direct the repair of the damaged DNA region, e.g., for homology directed repair (HDR).

The term “primary cell” refers to a cell isolated directly from a multicellular organism, such as a plant, a plantlet, an embryo, etc. Primary cells typically have undergone very few population doublings and are therefore more representative of the main functional component of the tissue from which they are derived in comparison to continuous (artificially immortalized) cell lines. In some cases, primary cells are cells that have been isolated and then used immediately. In other cases, primary cells cannot divide indefinitely and thus cannot be cultured for long periods of time in vitro.

The term “gene modified primary cell” or “genome edited primary cell” or “gene-edited cell” refers to a cell, e.g., a primary cell, into which a heterologous nucleic acid has been introduced in some cases, into its endogenous genomic DNA. Cultured cells, immortalized cells, and cell lines may also be gene-edited.

### CRISPR/Nuclease Gene Editing

There are provided herein CRISPR/nuclease (e.g., CRISPR/Cas) systems and methods for genome editing in Cannabis plant. The clustered regularly interspaced short palindromic repeats (CRISPR) genome-editing system is a two-component complex comprising a site-specific nuclease enzyme and one or more guide RNA. Generally, the guide RNA binds to the nuclease enzyme and specifies a target sequence within the genome. The nuclease enzyme cuts the two strands of DNA at a specific location in the genome defined by the guide RNA sequence, creating a double-stranded break (DSB) in the DNA. The DSB is then repaired by one of the cellular DNA-repair mechanisms.

Non-homologous end joining (NHEJ) is the dominant and most active DNA repair pathway in eukaryotes for repairing DSBs. NHEJ is error prone and results in mutations such as small insertions, deletions, and substitutions at the DSB site. CRISPR systems relying on NHEJ are commonly used to generate knockout mutants, e.g., by single gene editing.

Homologous recombination (HR) is a genetic process in which nucleotide sequences are exchanged between two similar molecules of DNA. HR is used by cells to accurately repair DSBs as well as other breaks that generate overhanging sequences. Homology-directed repair (HDR) is a mechanism in cells to accurately and precisely repair DSBs, using a homologous DNA template to guide repair via HR. HDR may result in an alteration of the sequence of the target molecule (e.g., insertion, deletion, mutation), if the sequence of the homologous DNA template differs from the target sequence and part or all of the DNA template is incorporated into the target DNA. In contrast to NHEJ, HDR is considered as an error-free process and is used to introduce specific desired modifications, e.g., knock-in, protein-domain swapping, new gene functions, or alterations in gene regulation.

In some embodiments of the present technology, the CRISPR/endonuclease system generates a DSB at the target DNA, which is then repaired using the cellular NHEJ mechanism.

In some embodiments of the present technology, the CRISPR/endonuclease system is used with a homologous DNA template which is also introduced into the cells, to generate specific genomic modifications via HDR.

CRISPR/nuclease systems and methods of the present technology can be used for a wide variety of genomic modifications. For example, and without limitation, the systems and methods can be used for: insertion, deletion, and/or substitution of one or more nucleotide at a target sequence; gene knockout; gene knockin; protein-domain swapping; introduction of new gene functions; modification of gene regulation; and the like. Such modifications may be used to provide, for example and without limitation: trait and/or crop improvement, e.g., nutritional enhancement, higher yields, improved stress tolerance, disease resistance, and the like. Further, CRISPR/nuclease systems provide genetically edited plants with desirable characteristics that are distinct from genetically modified organisms (GMOs) made using conventional techniques. Because CRISPR/nuclease gene editing methods may be considered similar to conventional breeding methods and natural biological evolution (Turnbull et al., 2021), plants made using such methods may benefit from different regulatory and legal status than GMOs.

Gene-editing nucleic acids may be introduced into cells using any suitable methods. In some embodiments, plant transformation methods are used for delivery of exogenous gene-editing nucleic acids into a cell. Any suitable method of transformation may be used, including for example and without limitation, protoplast transfection with polyethylene glycol (PEG) or via electroporation; Agrobacterium-mediated transformation; bioballistic transformation; viral vector agroinfection; and the like. The choice of method of transformation is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995. A transformation method may result in integration of the exogenous nucleic acid into the cellular genome, or may be transient, meaning the exogenous nucleic acids are not integrated into the genome. It should be understood that the transformation method is not meant to be particularly limited.

Gene editing systems and methods of the present technology can be used for gene editing and genomic modification on any plant selected from the genus Cannabis. In one embodiment, the plant is selected from the species Cannabis sativa.

It is further understood that a Cannabis plant of the present disclosure may exhibit the characteristics of any chemotype. As used herein, the term “chemotype” refers to the cannabinoid chemical phenotype in individual Cannabis strains. In general, chemotype is primarily determined by, but not limited to, chemical ratios or predominance of CBD, THC, and CBG and/or their acid counterparts CBDA, THCA, and CBGA present in mature or semi-mature Cannabis flower. For example, Small and Beckstead assigned chemotypes based on ratios of THCA and CBDA: plants producing primarily THCA (Type I), CBDA (Type III) or both THCA and CBDA (Type II) (Small & Beckstead, 1973). Much rarer CBGA-dominant hemp plants were later identified as a new chemotype (Type IV) (Fournier et al., 1987). Cannabis with less than 0.3% total THC by dry weight is recognized as hemp. In some embodiments of the present technology, the Cannabis strain has a low-THC/high-CBD chemotype, e.g., having a tetrahydrocannabinol (THC) content of between about 0.05% and about 0.25% by weight and/or a cannabidiol (CBD) content of between about 0.01% and about 10% by weight.

The present disclosure also provides for genetically modified parts of the Cannabis plants of the present disclosure. Plant parts, without limitation, include seed, endosperm, ovule and pollen. In one embodiment of the present disclosure, the plant part is a seed. In another embodiment of the present disclosure, the plant part is a plant cell.

In some embodiments, the present technology also provides for genetically modified Cannabis organisms, tissues or cells such as Cannabis plants, Cannabis tissue and Cannabis cells having at least one gene edit or genomic modification generated using the systems and methods of the present technology.

In some embodiments, the present technology also provides for genetically modified Cannabis organisms, tissues or cells that comprise the genetically modified Cannabis plant cells as described herein. In some embodiments, the genetically modified organisms, tissues or cells are plants, plant tissues or plant cells that exhibit one or more desired property conferred by gene editing. In some instances, such plants are Cannabis plants and such plant tissues and plant cells are Cannabis tissue and Cannabis cells.

### Guide RNAs

The RNA molecule that binds to the site-specific nuclease and targets the nuclease to a specific target location within a target DNA or target gene is referred to herein as a “guide RNA”. The terms “guide RNA”, “gRNA”, and “guide” are used interchangeably herein. A guide RNA typically comprises two segments, a “DNA-targeting segment” and a “nuclease-binding segment.” By “segment” is meant a segment, section, or region of a molecule, e.g., a contiguous stretch of nucleotides in an RNA. A segment can also mean a region or section of a complex such that a segment may comprise regions of more than one molecule. For example, in some cases the nuclease-binding segment (described below) of a guide RNA is one RNA molecule and the nuclease-binding segment therefore comprises a region of that RNA molecule. In other cases, the nuclease-binding segment (described below) of a guide RNA comprises two separate molecules that are hybridized along a region of complementarity. As an illustrative, non-limiting example, a nuclease-binding segment of a guide RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and may include regions of RNA molecules that are of any total length and may or may not include regions with complementarity to other molecules.

The DNA-targeting segment (or “DNA-targeting sequence”) comprises a nucleotide sequence that is complementary to a specific sequence within a target DNA sequence or target genomic DNA (gDNA) region (the complementary strand of the target DNA); this region is designated the “protospacer-like” sequence herein. The nuclease-binding segment (or “nuclease-binding sequence”) interacts with a nuclease. When the nuclease is a Cas9 or Cas9 related enzyme (described in more detail below), site-specific cleavage of the target DNA occurs at locations determined by both (i) base-pairing complementarity between the guide RNA and the target DNA; and (ii) a short motif (referred to as the protospacer adjacent motif, or “PAM”) in the target DNA sequence or “target gDNA” or “target gene”.

As used herein, the terms “target genomic DNA (gDNA)”, “target genomic DNA (gDNA) region”, “gDNA” and “target gene” are used interchangeably to refer to a target DNA sequence present in the genome of a cell, i.e., a chromosomal target DNA sequence. In some cases, where it is clear that the target DNA sequence is present in the genome of a cell, the terms “target DNA sequence” and “target genomic DNA (gDNA) region” and “gDNA” and “target gene” may be used interchangeably.

The nuclease-binding segment of a guide RNA comprises, in part, two complementary stretches of nucleotides that hybridize to one another to form a double stranded RNA duplex (dsRNA duplex).

In some embodiments of the present technology, a nucleic acid (e.g., a guide RNA, a nucleic acid comprising a nucleotide sequence encoding a guide RNA; a nucleic acid encoding a nuclease enzyme; etc.) comprises a modification or sequence that provides for an additional desirable feature (e.g., modified or regulated stability; subcellular targeting; tracking, e.g., a fluorescent label; a binding site for a protein or protein complex; etc.). Non-limiting examples include: a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

In some embodiments, a guide RNA comprises an additional segment at either the 5′ or 3′ end that provides for any of the features described above. For example, a suitable third segment can comprise a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and protein complexes); a stability control sequence; a sequence that forms a dsRNA duplex (i.e., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

A guide RNA and a nuclease enzyme bind and form a ribonucleoprotein complex via non-covalent interactions. The guide RNA provides target specificity to the complex by comprising a nucleotide sequence that is complementary to a sequence of a target DNA. The nuclease of the complex provides the site-specific cleavage activity. In other words, the nuclease is guided to the target DNA sequence (e.g. a target sequence in a chromosomal nucleic acid, e.g., a genome; a target sequence in an extrachromosomal nucleic acid, e.g. an episomal nucleic acid, a minicircle, etc.; a target sequence in a mitochondrial nucleic acid; a target sequence in a chloroplast nucleic acid; a target sequence in a plasmid; etc.) by virtue of its association with the nuclease-binding segment of the guide RNA.

In some embodiments, a guide RNA comprises two separate RNA molecules (RNA polynucleotides: an “crRNA” and a “tracrRNA”) and may be referred to herein as a “double-molecule guide RNA” or a “two-molecule guide RNA.” In other embodiments, the guide RNA is a single RNA molecule (single RNA polynucleotide) and may be referred to herein as a “single guide RNA” or an “sgRNA.” The term “guide RNA” or “gRNA” is inclusive, referring both to double-molecule guide RNAs and to single-molecule guide RNAs (i.e., sgRNAs). An exemplary single guide RNA comprises a CRISPR RNA (crRNA or crRNA-like) molecule which includes a CRISPR repeat or CRISPR repeat-like sequence and a corresponding trans-activating crRNA (tracrRNA or tracrRNA-like) molecule. A crRNA molecule comprises both the DNA-targeting segment (single stranded) of the guide RNA and a stretch (a duplex-forming segment) of nucleotides that forms one half of the dsRNA duplex of the nuclease-binding segment of the guide RNA. The corresponding tracrRNA molecule comprises a stretch of nucleotides (a duplex-forming segment) that forms the other half of the dsRNA duplex of the protein-binding segment of the guide RNA. In other words, a stretch of nucleotides of the crRNA molecule are complementary to and hybridize with a stretch of nucleotides of the tracrRNA molecule to form the dsRNA duplex of the nuclease-binding domain of the guide RNA. As such, each crRNA molecule can be said to have a corresponding tracrRNA molecule. The crRNA molecule additionally provides the single stranded DNA-targeting segment. Thus, a crRNA and a tracrRNA molecule (as a corresponding pair) hybridize to form a guide RNA. A double-molecule guide RNA can comprise any corresponding crRNA and tracrRNA pair.

A single guide RNA comprises two stretches of nucleotides (a crRNA and a tracrRNA) that are complementary to one another, are covalently linked (directly, or by intervening nucleotides), and hybridize to form the double stranded RNA duplex (dsRNA duplex) of the nuclease-binding segment, thus resulting in a stem-loop structure. The crRNA and the tracrRNA can be covalently linked via the 3′ end of the crRNA and the 5′ end of the tracrRNA. Alternatively, crRNA and the tracrRNA can be covalently linked via the 5′ end of the crRNA and the 3′ end of the tracrRNA. An sgRNA can comprise any corresponding crRNA and tracrRNA sequences.

In some embodiments, where the nuclease enzyme does not require the tracrRNA sequence (e.g., as in the case of the Cas12a enzyme), the guide RNA and/or the sgRNA can comprise a crRNA alone, or multiple crRNAs, without the tracrRNA.

### CRISPR Endonucleases

CRISPR endonucleases are CRISPR-associated site-specific nucleases that cleave DNA at sites specified by the guide RNA.

The CRISPR-associated protein 9 (“Cas9”) enzyme from Streptococcus pyogenes (SpCas9) is predominantly used in many CRISPR/nuclease systems (i.e., CRISPR/Cas systems). However, many different CRISPR-associated proteins have been identified in a variety of bacteria and archaea and can be used in the systems and methods of the present technology. Non-limiting examples of CRISPR endonucleases for use in accordance with the present technology include: the Staphylococcus aureus Cas9 (SaCas9), Staphylococcus auricularis Cas9 (SauriCas9), Streptococcus thermophilus Cas9 (StCas9), Neisseria meningitidis Cas9 (NmCas9), Francisella novicida Cas9 (FnCas9), Acidaminococcus Cas12a (AsCas12a), Francisella novicida Cas12a (FnCas12a), Lachnospiraceae bacterium Cas12a (LbCas12a), Francisella novicida Cas12a (also known as “Cpf1” and “FnCas12a”), Brevibacillus laterosporus (BlatCas9), Campylobacter jejuni (CjCas9), Leptotrichia shahii Cas13a (also known as “LshCas13a” and “Cas13a”), Deltaproteobacteria CasX (DpbCasX), CasY (also known as “Cas12d”), and Mtube Protein 1 (Csm1). In addition, Cas9 variants that have been engineered to modulate the enzymatic properties, for example cleavage (creating a single-strand rather than a double-strand break, i.e. a “nick”) (Cas9n), fidelity enhanced nuclease (eSpCas9), hyper-accurate Cas9 (HypaCas9), and the like, may also be used in the systems and methods of the present technology.

In certain embodiments, the nucleic acid encoding the CRISPR endonuclease is plant codon optimized.

In certain embodiments, the CRISPR endonuclease is linked to a nuclear localization signal or sequence, for translocation to the nucleus. Any suitable nuclear localization signal may be used and the NLS is not meant to be particularly limited. For example, and without limitation, the Simian virus 40 (SV40) T antigen nuclear localization sequence may be used. The NLS may be linked to the N- or C-terminal of the endonuclease. In some cases, it may be advantageous to link the nuclease to more than one NLS to increase the efficiency of editing.

The different CRISPR endonucleases and their associated CRISPR/nuclease gene-editing systems often have different gene-editing characteristics. For example, the Cas9 enzyme requires a single guide RNA (sgRNA) derived from the fusion of CRISPR RNA (crRNA) and trans-activating crRNA (tracrRNA). In contrast, the Cas12a enzyme requires only small CRISPR-derived RNA (crRNAs, 42-44-nt per-crRNA, 19-nt repeat and 23-25-nt spacer). Multiple crRNAs can be expressed as a single transcript to generate functional individual crRNAs after processing through Cas12a nuclease; this has been shown to increase the efficiency of crRNA entry into cells (Fagerlund et al., 2015; Nakade et al., 2017). Cas12a nuclease also generates a 5-bp staggered DNA double-strand break ends that are formed downstream of the PAM sequence, while the Cas9 nuclease only forms a blunt-end cut 3 bp upstream of the PAM sequence. Another major difference between the Cas12a and Cas9 systems is that Cas12a recognizes a T-rich protospacer-adjacent motif (PAM), while Cas9 recognizes a G-rich PAM (Fonfara et al., 2016). The Cas9 and Cas12a CRISPR systems are thus useful for gene editing at different target sites, with Cas9 particularly useful for G-C rich target sites, and Cas12a for A-T rich target sites. The CRISPR endonuclease for use in systems and methods of the present technology will therefore be selected based on several factors, including the target sequence; the type of modification desired; and the like, and is not meant to be particularly limited.

### Additional Exemplary Embodiments

Additional exemplary embodiments are provided herein, for illustration.

Embodiment 1. A method for genetically editing callus cells from a Cannabis plant, said method comprising: transforming the callus cells with Agrobacterium cells carrying an exogenous gene editing DNA sequence; and selecting transformed callus cells which are gene-edited by the exogenous gene-editing DNA sequence.

Embodiment 2. The method of Embodiment 1, wherein the exogenous gene-editing DNA sequence is for editing one of CBDAS, CBCAS, R3-MYB1, R3-MYB2, R3-MYB3, R3-MYB4, and eIF4e in the callus cells.

Embodiment 3. The method of Embodiment 1 or 2, wherein the exogenous DNA sequence comprises a DNA sequence encoding for a Cas enzyme.

Embodiment 4. The method of Embodiment 3, wherein the Cas enzyme is Cas9 or Cas12.

Embodiment 5. A genetically modified callus cell obtained by the method of any one of Embodiments 1 to 4.

Embodiment 6. A genetically modified Cannabis plant, plant part, tissue or cell thereof, generated from the gene-edited callus cells obtained by the method of any one of Embodiments 1 to 4.

Embodiment 7. A gene-edited Cannabis plant comprising a gene-edited CBDAS, CBCAS, R3-MYB1, R3-MYB2, R3-MYB3, R3-MYB4, or eIF4e gene.

Embodiment 8. A method for genetically editing a somatic cell of a Cannabis somatic embryo, said method comprising: transforming the somatic cell with Agrobacterium cells carrying an exogenous gene editing DNA sequence; and selecting somatic cells which are gene-edited by the exogenous gene editing DNA sequence.

Embodiment 9. The method of Embodiment 8, wherein the cells of the Cannabis somatic embryo are immortalized.

Embodiment 10. The method of Embodiment 8 or 9, wherein the Cannabis somatic embryo is a compact callus capable of forming somatic embryos.

Embodiment 11. The method of Embodiment 8 or 9, wherein the exogenous gene-editing DNA sequence is for editing one of CBDAS, CBCAS, R3-MYB1, R3-MYB2, R3-MYB3, R3-MYB4, and eIF4e in the somatic cell.

Embodiment 12. The method of any one of Embodiments 8 to 11 further comprising generating a transgenic Cannabis plant from the gene-edited somatic cells.

Embodiment 13. The method of Embodiment 12, wherein the gene-edited somatic cells are gene-edited male founder somatic cells.

Embodiment 14. The method of Embodiment 13, further comprising generating a female Cannabis plant from the gene-edited male founder somatic cells.

Embodiment 15. A genetically modified Cannabis somatic cell obtained by the method of any one of Embodiments 8 to 14.

Embodiment 16. A method for producing at least one Cannabis somatic embryo comprising: (a) culturing a Cannabis explant in a first medium comprising an auxin to form roots; (b) culturing the roots in a second medium comprising a first cytokinin to induce formation of callus; and (c) culturing the callus in a third medium comprising a second cytokinin to produce the at least one Cannabis somatic embryo.

Embodiment 17. The method of Embodiment 16, wherein the Cannabis explant is obtained from shoots, leaves, stems, flowers, and/or roots.

Embodiment 18. The method of Embodiment 16, wherein the Cannabis explant is a hypocotyl, cotyledon, or a mixture thereof.

Embodiment 19. The method of any one of Embodiments 16 to 18, wherein the auxin is indole-3-acetic acid (IAA), 4-chloroindole-3-acetic acid (4-Cl-IAA), phenylacetic acid (PAA), indole-3-butyric acid (IBA), indole-3-propionic acid (IPA), indole-3-pyrruvic acid (IPyA), indole-3-acetamide (IAM), indole-3-ethanol (IAEt), indole-3-acetaldehyde (IAAld), indole-3-acetonitrile (IAN), tryptophan (TRP), tryptamine (TRA), 1-naphthaleneacetic acid (NAA), 2,4-dichlorophenoxyacetic acid (2,4-D), 3,6-dichloro-2-methoxybenzoic acid (Dicamba), 4-amino-3,5,6-trichloropyridine-2-carboxylic acid (Picloram), or any combination thereof.

Embodiment 20. The method of any one of Embodiments 16 to 19, wherein the first cytokinin is an adenine-type cytokinin, a phenylurea-type cytokinin, or a combination thereof.

Embodiment 21. The method of any one of Embodiments 16 to 19, wherein the first cytokinin is wherein the first cytokinin is Nisopentenyladenine (iP), N6-isopentenyladenosine (iPR), N6-isopentenyladenine-7-glucoside (iP7G), N6-isopentenyladenine-9-glucoside (iP9G), N6-isopentenyladenosine-5′-monophosphate (iPRMP), N6-isopentenyladenosine-5′-diphosphate (iPRDP), N6-isopentenyladenosine-5′-triphosphate (iPRTP), trans-zeatin (tZ), trans-zeatin glucoside (tZR), trans-zeatin-7-glucoside (tZ7G), trans-zeatin-9-glucoside (tZ9G), trans-zeatin 0-glucoside (tZOG), trans-zeatin riboside O-glucoside (tZROG), trans-zeatin riboside-5′-monophosphate (tZRMP), trans-zeatin riboside-5′-diphosphate (tZRDP), trans-zeatin riboside-5′-triphosphate (tZRTP), cis-zeatin (cZ), cis-zeatin glucoside (cZR), cis-zeatin-9-glucoside (cZ9G), cis-zeatin O-glucoside (cZOG), cis-zeatin riboside O-glucoside (cZROG), cis-zeatin riboside-5′-monophosphate (cZRMP), cis-zeatin riboside-5′-diphosphate (cZRDP), cis-zeatin riboside-5′-triphosphate (cZRTP), N6-benzyladenine (BA), N6-benzyladenosine (BAR), N6-benzyladenine-3-glucoside (BA3G), N6-benzyladenine-7-glucoside (BA7G), N6-benzyladenine-9-glucoside (BA9G), N6-benzyladenosine-5′-monophosphate (BARMP), N6-benzyladenosine-5′-diphosphate (BARDP), N6-benzyladenosine-5′-triphosphate (BARTP), dihydrozeatin (DHZ), dihydrozeatin glucoside (DHZR), dihydrozeatin-9-glucoside (DHZ9G), dihydrozeatin O-glucoside (DHZOG), dihydrozeatin riboside O-glucoside (DHZROG), dihydrozeatin riboside-5′-monophosphate (DHZRMP), dihydrozeatin riboside-5′-diphosphate (DHZRDP), dihydrozeatin riboside-5′-triphosphate (DHZRTP), ortho-topolin (oT), ortho-topolin riboside (oTR), ortho-topolin-9-glucoside (oT9G), meta-topolin (mT), meta-topolin riboside (mTR), meta-topolin-9-glucoside (mT9G), para-topolin (pT), para-topolin riboside (pTR), kinetin (K), kinetin riboside (KR), kinetin-9-glucoside (K9G), diphenylurea (DPU), thidiazuron (TDZ), or any combination thereof.

Embodiment 22. The method of any one of Embodiments 16 to 21, wherein the second cytokinin is an adenine type cytokinin, a phenylurea-type cytokinin, or a combination thereof.

Embodiment 23. The method of any one of Embodiments 16 to 21, wherein the second cytokinin is Nisopentenyladenine (iP), N⁶-isopentenyladenosine (iPR), N⁶-isopentenyladenine-7-glucoside (iP7G), N⁶-isopentenyladenine-9-glucoside (iP9G), N⁶-isopentenyladenosine-5′-monophosphate (iPRMP), N⁶-isopentenyladenosine-5′-diphosphate (iPRDP), N⁶-isopentenyladenosine-5′-triphosphate (iPRTP), trans-zeatin (tZ), trans-zeatin glucoside (tZR), trans-zeatin-7-glucoside (tZ7G), trans-zeatin-9-glucoside (tZ9G), trans-zeatin O-glucoside (tZOG), trans-zeatin riboside O-glucoside (tZROG), trans-zeatin riboside-5′-monophosphate (tZRMP), trans-zeatin riboside-5′-diphosphate (tZRDP), trans-zeatin riboside-5′-triphosphate (tZRTP), cis-zeatin (cZ), cis-zeatin glucoside (cZR), cis-zeatin-9-glucoside (cZ9G), cis-zeatin O-glucoside (cZOG), cis-zeatin riboside O-glucoside (cZROG), cis-zeatin riboside-5′-monophosphate (cZRMP), cis-zeatin riboside-5′-diphosphate (cZRDP), cis-zeatin riboside-5′-triphosphate (cZRTP), N⁶-benzyladenine (BA), N⁶-benzyladenosine (BAR), N⁶-benzyladenine-3-glucoside (BA3G), N⁶-benzyladenine-7-glucoside (BA7G), N⁶-benzyladenine-9-glucoside (BA9G), N⁶-benzyladenosine-5′-monophosphate (BARMP), N⁶-benzyladenosine-5′-diphosphate (BARDP), N⁶-benzyladenosine-5′-triphosphate (BARTP), dihydrozeatin (DHZ), dihydrozeatin glucoside (DHZR), dihydrozeatin-9-glucoside (DHZ9G), dihydrozeatin O-glucoside (DHZOG), dihydrozeatin riboside O-glucoside (DHZROG), dihydrozeatin riboside-5′-monophosphate (DHZRMP), dihydrozeatin riboside-5′-diphosphate (DHZRDP), dihydrozeatin riboside-5′-triphosphate (DHZRTP), ortho-topolin (oT), ortho-topolin riboside (oTR), ortho-topolin-9-glucoside (oT9G), meta-topolin (mT), meta-topolin riboside (mTR), meta-topolin-9-glucoside (mT9G), para-topolin (pT), para-topolin riboside (pTR), kinetin (K), kinetin riboside (KR), kinetin-9-glucoside (K9G), diphenylurea (DPU), thidiazuron (TDZ), or any combination thereof.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1—In Vitro Screen of Cas9 and Cas12a Cut Sites and Selection of Guide RNA SEQUENCES

Cas9 and Cas12a in vitro digestion assays were performed against Cannabis gene sequences using synthetic guide RNAs (IDT) and either ALT-R Cas9-ribonucleoprotein (RNP) complexes (IDT) or EnGen Lba Cas12a (Cpf1)-RNP complexes (New England Biolabs) to find efficient guides for in vivo use. Target gene bands for cleavage were amplified from Cannabis genomic DNA using gene-specific primers and Q5 DNA polymerase (NEB). For the in vitro digestion assays, Cas9 guide RNAs were developed for the CBDAS, CBCAS, R3-MYB1, R3-MYB2, R3-MYB3, R3-MYB4 and eIF4e genes, and Cas12a guide RNAs were developed for the CBDAS, CBCAS, R3-MYB1 and R3-MYB2 genes.

CBCAS and THCAS. Because of the sequence similarity between CBCAS and THCAS, guide RNAs were designed to target sequence regions that are conserved between these two synthases; these guide RNAs are referred to as either CBCAS- or THCAS-targeting or both.

R3-MYB2. R3-MYB2 was found to contain two isoforms named R3-MYB2a and R3-MYB2b in some genotypes.

Guide RNAs (also referred to herein as “guides”) labeled with the prefix “MP” refer to guide RNAs specific for Cas9, i.e., targeting sequences next to the Cas9 PAM and therefore designed for use with Cas9. Guide RNAs labeled with the prefix “LB” refer to guide RNAs specific for Cas12a, e.g., targeting sequences next to the Cas12a PAM and therefore designed for use with Cas12a.

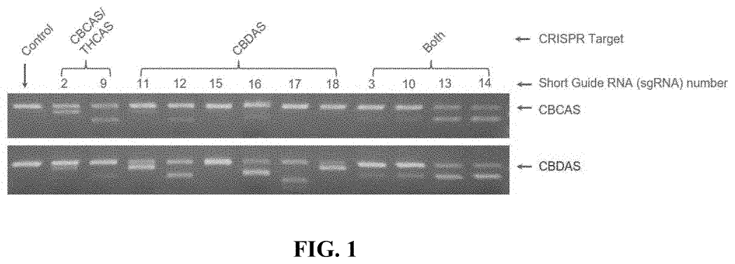

CBDAS and CBCAS—Cas9. Guide 18 was determined to be selective for targeting of CBDAS over other synthases. Guides 13 and 14 were found to be pan-cutters at all tested cannabinoid synthase target genes, i.e., both CBDAS and CBCAS. Guide 9 was determined to be selective for CBCAS and THCAS. Results are shown in FIG. 1. These guide sequences were cloned into vectors for in vivo use with Cas9.

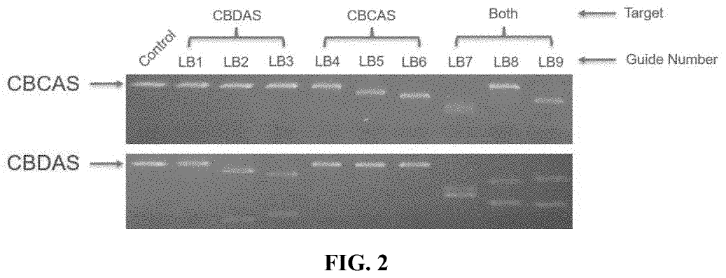

CBDAS and CBCAS—Cas12a. Guides 2 and 3 were determined to be selective for CBDAS. Guide 6 was determined to be selective for CBCAS. Guide 7 was found to be a pan-cutter (i.e. both synthases were cut). Results are shown in FIG. 2. These guide sequences were cloned into vectors for in vivo use with Cas12a.

R3-MYBs. 4 R3-MYB genes were identified in a hemp genome. Cas9 guides were designed for each of R3-MYBs 1-4, and Cas12a guides were designed for R3-MYB1 and 2.

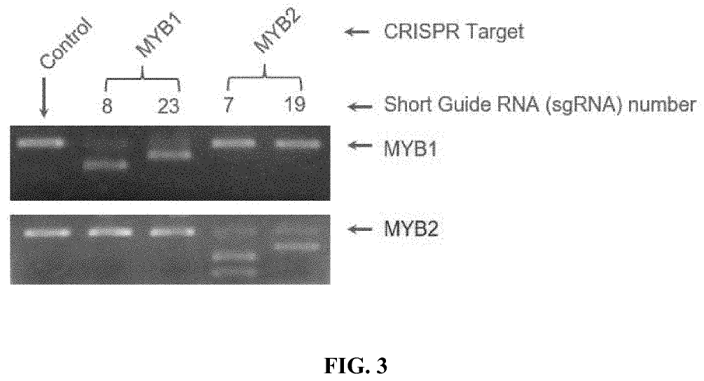

For the Cas9 guides, Guides 8 and 23 were determined to be specific for targeting R3-MYB1 versus R3-MYB2. Guides 7 and 19 were determined to be specific for R3-MYB2 versus R3-MYB1. Results are shown in FIG. 3. These guide sequences were cloned into vectors for in vivo use with Cas9.

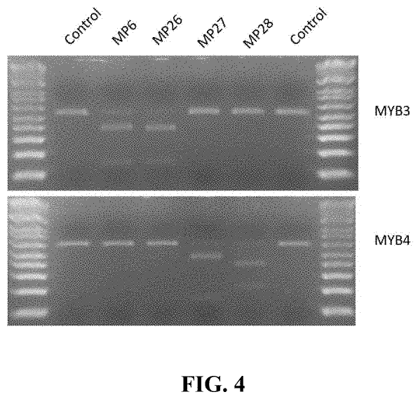

Guides 6 and 26 were determined to be specific for MYB3, and Guides 27 and 28 were determined to be specific for MYB4. Results are shown in FIG. 4. These guide sequences were cloned into vectors for in vivo use with Cas9.

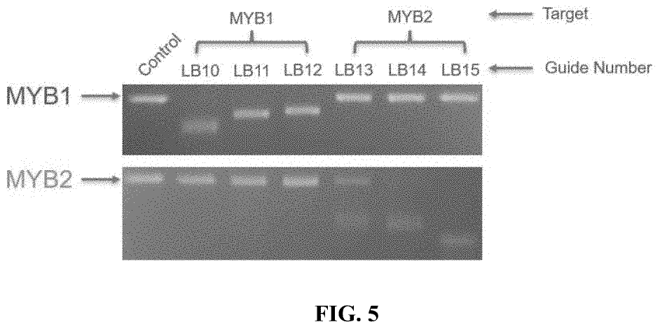

For the Cas12a guides, Guide 12 was determined to be specific for MYB and Guide 15 was determined to be specific for MYB2. Results are shown in FIG. 5. These guide sequences were cloned into vectors for in vivo use with Cas2a.

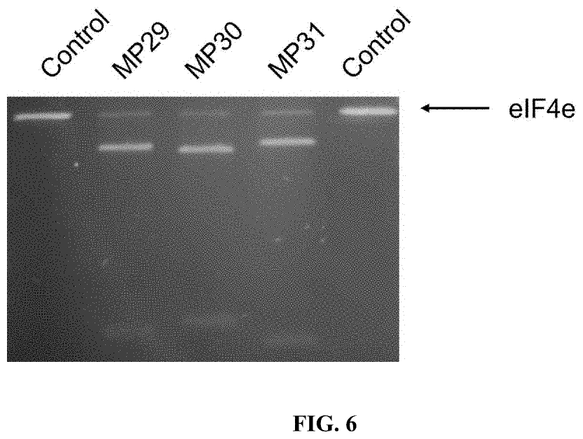

eIF4e. For Cas9 guides, Guides 29 and 30 were determined to be efficient for eF4e. Results are shown in FIG. 6. These guide sequences were cloned into vectors for in vivo use with Cas9.

Cas9 guide sequences are shown in Table 1. Cas12a guide sequences are shown in Table 2. All sequences are shown in the 5′ to 3′ direction from left to right. For each guide, the tables show: the Target gene; the Guide number; the guide Sequence (note the sequences shown are the DNA sequences corresponding to the guide RNAs); which strand of the DNA (plus or minus) is targeted by the guide; and the SEQ TD No.

TABLE 1
Exemplary Cas9 guide sequences.

| Target | Guidenumber | Sequence | Strand | SEQ ID NO: |
|---|---|---|---|---|
| THCAS/CBCAS | MP2 | GAAGGAGTGACAATAACGAG | minus | 1 |
| | MP9 | GTAGGTGGACACTTTAGTGG | plus | 2 |
| CBDAS | MP11 | GAAGGAGTGACGATAACAAG | minus | 3 |
| | MP12 | GCAGGTGGACACTTTGGTGG | plus | 4 |
| | MP15 | GTTTTCTCGAGGATTAGCAA | minus | 5 |
| | MP16 | CCTTGGAGAAGTTTATTATT | plus | 6 |
| | MP17 | GGTGGTGGAGCAGAAAGCTT | plus | 7 |
| | MP18 | GTGACGATAACAAGTGGTTT | minus | 8 |
| All Cannabinoid | MP3 | AACTTCTCCAAGGGTAGCTC | minus | 9 |
| Synthases | MP10 | GTTGAAGCCGGAGCTACCCT | plus | 10 |
| | MP13 | CCCTTGGAGAAGTTTATTAT | plus | 11 |
| | MP14 | CCAATAATAAACTTCTCCAA | minus | 12 |
| CsR3-MYB1 | MP8 | AATTAGGCGTGAAGTAGTTG | plus | 13 |
| | MP23 | TGAAGGTGGTCACTTATTGC | plus | 14 |
| | MP24 | ACTTATTGCTGGAAGAATTC | plus | 15 |
| | MP25 | CTTATTGCTGGAAGAATTCC | plus | 16 |
| CsR3-MYB2 | MP7 | ATGAAGAAGCCCTTATTACT | plus | 17 |
| | MP19 | ATTAACGGAAGAATAAGAAG | minus | 18 |
| | MP20 | TATTCTTCCGTTAATTCAAC | plus | 19 |
| | MP21 | TAGGATGTTTAATCTTGTGG | plus | 20 |
| CsR3-MYB3 | MP6 | ACACCACCCCAATTCGTCGC | plus | 21 |
| | MP26 | TATAGAATGTTTTCACTCGT | plus | 22 |
| CsR3-MYB4 | MP27 | TGGGTTCTTCATTATTTCCT | minus | 23 |
| | MP28 | TTTAGAATGTACAAGCTCGT | plus | 24 |
| elF4e | MP29 | AAGGTCCAAGAATGTTCGAG | minus | 25 |
| | MP30 | GGAAGGGTTATCGAACCAAA | minus | 26 |
| | MP31 | GGTGAGGTTGATGGAGTAAG | minus | 27 |

TABLE 2
Exemplary Cas12a guide sequences.

| Target | Guide number | Guide Sequence | Strand | SEQ ID NO: |
|---|---|---|---|---|
| CBDAS | LB1 | TTGCATTATTGGGAATATATTGC | minus | 28 |
| | LB2 | ATTGAACGCATGTTTCTCAAGTC | minus | 29 |
| | LB3 | TCATTAACCCAATAATAAACTTC | minus | 30 |
| CBCAS | LB4 | CTGGATTGTTAGGAATATATTCC | minus | 31 |
| | LB6 | TCCTACATATCTCAAGTCCCATT | plus | 32 |
| | LB5 | ACCGTATGCATGTTTCTCAAGTC | minus | 33 |
| All cannabinoid | LB7 | TCATACTTGTAAGCAATATTTTG | minus | 34 |
| synthases | LB8 | CTTGGTGGAGTGGATAGTCTAGT | plus | 35 |
| | LB9 | CTGAGTTGGGTATTAAAAAAACG | plus | 36 |
| CsR3-MYB1 | LB10 | TCCAATTAGATTGAACATTCTGA | minus | 37 |
| | LB11 | CTCTGCTGTTCTTCCCGGAATTC | minus | 38 |
| | LB12 | ATACCCAATACTTCTCAATTTCC | minus | 39 |
| CsR3-MYB2 | LB13 | AGCTTGATTTTTCAGAAGATGAA | plus | 40 |
| | LB14 | AGAAGATGAAGAAGCCCTTATTA | plus | 41 |
| | LB15 | CCCCACAAGATTAAACATCCTAG | minus | 42 |

### Example 2—Plasmids for Expressing CRISPR Cas Components

Vectors with guide RNAs and gene overexpression SpCas9-NLS was cloned under the CaMV 35s n vectors were developed for use in gene editing. Such vectors are referred to herein as “editing vectors”.

“SpCas9” refers to Streptococcus pyogenes Cas 9. “NLS” refers to Simian virus 40 (SV40) T antigen nuclear localization sequence (NLS), which is fused to SpCas9 to form SpCas9-NLS. SpCas9-NLS is an RNA-guided endonuclease that catalyzes site-specific cleavage of double stranded DNA. The location of the cleavage is within the target sequence 3 bases from the Protospacer Adjacent Motif (PAM), which is NGG for the SpCas9 enzyme. The PAM sequence, NGG, must follow the targeted region on the opposite strand of the DNA with respect to the region-complementary guide sequence for the enzyme to cleave the DNA.

For Cas9 editing vectors, SpCas9-NLS was cloned under the CaMV 35s promoter, and a hygromycin cassette was used to provide hygromycin resistance. Specifically, SpCas9-NLS was cloned into multiple cloning site (MCS) 1 (MCS1) of pRI201-AN under the CaMV 35s promoter. An AtU6 promoter cassette was cloned into MCS2, and Nos-promoted NPT II for kanamycin resistance was swapped with an additional CaMV 35s promoted hygromycin phosphotransferase to provide hygromycin resistance. Cas9-specific guide RNA sequences as selected above were inserted into this vector. Guides were cloned under an Arabadopsis thaliana U6 promoter to create pRI-Cas9 series vectors that were compatible for both Agrobacterium-mediated and biolistic transformation. An exemplary vector is shown schematically in FIG. 19.

Because Agrobacterium-specific sequences are a large fraction of Ti and Ri vectors, for biolistic transformation the guide RNA blocks between the first 35S and Nos terminator for both Cas9 and Cas12a constructs were cloned into the minimal backbone of pUC19, to make pUC-Cas9 and pUC-Cas12a series vectors. All series vectors were used for transformation, editing, and Hygromycin selection.

For gene overexpression vectors, a 35s promoter followed by a multiple cloning site and a hygromycin cassette was developed. Coding sequences for green fluorescent protein (GFP) or Arabadopsis thaliana EGL3 (AtEGL3) were cloned into the multiple cloning site to make 35s:GFP and 35s:AtEGL3 vectors, respectively.

More details on vector construction are as follows:

A hygromycin resistant, agrobacterium compatible expression vector (pRI-HygroR) was developed by cloning a 35S::hph cassette in place of NOS::NPTII in pRI 201-AN (Takara Bio).

SpCas9-NLS was cloned into multiple cloning site 1 (MCS1) and AtU6 promoted guide (“Cas9 guideblock”) was cloned into multiple cloning site 2 (MCS2) to create the pRI-Cas9-HygroR vector. In the pRI-Cas9-HygroR vector and the Cas9 guideblock, the “nnnnnnnnnnnnnnnnnnnn” nucleotides indicate where the variable guide sequences were inserted. Guides beginning with G were cloned using the subsequent 19 nucleotides, otherwise the full 20 nucleotide sequence was cloned. The pRI-Cas9-HygroR vector is shown schematically in FIG. 19.

To make a smaller vector for biolistic transformation, the cassette including 35S::Cas9, the Cas9 guideblock and 35S::hph was cloned into pUC19, to make pUC-Cas9-HygroR. The “nnnnnnnnnnnnnnnnnnnn” nucleotides indicate where the variable guide sequences were inserted.

The region including the first 35S promoter and the 35S::hph cassette, or without the 35S::hph cassette, of pRI-HygroR were cloned into pUC19 to make cloning vectors pUC-HygroR and pUC, respectively. These vectors contained empty MCS1 and MCS2. pUC-HygroR allows gene expression in Cannabis of products cloned into MCS1 with integrative hygromycin resistance. pUC allows gene expression in Cannabis of products cloned into MCS1 without integrative hygromycin resistance.

Green fluorescent protein (GFP) coding sequence was cloned into MCS1 of pUC-HygroR and pUC to make pUC-GFP-HygroR and pUC-GFP, respectively. pUC-GFP-HygroR was used for biolistic transformations to make stably transformed lines constitutively expressing GFP under hygromycin selection. pUC-GFP was used in combination with other vectors for biolistic transformation and transient GFP expression.

The coding sequence of Enhancer of Glabrous 3 from Arabidopsis thaliana (AtEGL3) was cloned into MCS1 of pUC-HygroR to make pUC-AtEGL3-HygroR.

Cas9 guides (20 nucleotides) are shown in Table 1 above. For Cas9 guides cloned into pRI-Cas9-HygroR and/or pUC-Cas9-HygroR vectors, if the guide sequence begins with a G, then the following 19 nucleotides were cloned into the vector, otherwise the 20 nucleotide sequences were cloned into the variable region of the Cas9 guide block.

For Cas12a editing vectors, LbCas12a was cloned in place of SpCas9 in these vectors, under the CaMV 35s promoter, and a Cas12a specific guide block was cloned in MCS2, in place of the Cas9a specific guide block, under an Arabadopsis thaliana U6 promoter, to make pRI-Cas12a series vectors.

LbCas12a-NLS and an AtU6-promoted Cas12a-compatible guide block (“Cas12a guideblock”) were cloned in place of Cas9 and the Cas9 guideblock in pUC-Cas9-HygroR, respectively, to create pUC-Cas12a-HygroR. “nnnnnnnnnnnnnnnnnnnnn” nucleotides represent variable guide sequences of 21 nucleotides.

Cas12a guides (23 nucleotides) are shown in Table 2 above. For Cas12a guides cloned into pUC-Cas12a-HygroR vector, the first 21 nucleotides were cloned into the variable region of the Cas12a compatible guide block.

Editing vector sequences are shown in Table 3. All sequences are shown in the 5′ to 3′ direction from left to right.

TABLE 3
Exemplary editing vector sequences.

| Name | Sequence | SEQ ID NO: |
|---|---|---|
| pRI- | GTTGCCATGTTTTACGGCAGTGAGAGCAGAGATAGCGCTGATGTCCGGCGGTGCTTTTGC | 43 |
| HygroR | CGTTACGCACCACCCCGTCAGTAGCTGAACAGGAGGGACAGCTGATAGAAACAGAAGCCA | |
| | CTGGAGCACCTCAAAAACACCATCATACACTAAATCAGTAAGTTGGCAGCATCACCCATA | |
| | ATTGTGGTTTCAAAATCGGCTCCGTCGATACTATGTTATACGCCAACTTTGAAAACAACT | |
| | TTGAAAAAGCTGTTTTCTGGTATTTAAGGTTTTAGAATGCAAGGAACAGTGAATTGGAGT | |
| | TCGTCTTGTTATAATTAGCTTCTTGGGGTATCTTTAAATACTGTAGAAAAGAGGAAGGAA | |
| | ATAATAAATGGCTAAAATGAGAATATCACCGGAATTGAAAAAACTGATCGAAAAATACCG | |
| | CTGCGTAAAAGATACGGAAGGAATGTCTCCTGCTAAGGTATATAAGCTGGTGGGAGAAAA | |
| | TGAAAACCTATATTTAAAAATGACGGACAGCCGGTATAAAGGGACCACCTATGATGTGGA | |
| | ACGGGAAAAGGACATGATGCTATGGCTGGAAGGAAAGCTGCCTGTTCCAAAGGTCCTGCA | |
| | CTTTGAACGGCATGATGGCTGGAGCAATCTGCTCATGAGTGAGGCCGATGGCGTCCTTTG | |
| | CTCGGAAGAGTATGAAGATGAACAAAGCCCTGAAAAGATTATCGAGCTGTATGCGGAGTG | |
| | CATCAGGCTCTTTCACTCCATCGACATATCGGATTGTCCCTATACGAATAGCTTAGACAG | |
| | CCGCTTAGCCGAATTGGATTACTTACTGAATAACGATCTGGCCGATGTGGATTGCGAAAA | |
| | CTGGGAAGAAGACACTCCATTTAAAGATCCGCGCGAGCTGTATGATTTTTTAAAGACGGA | |
| | AAAGCCCGAAGAGGAACTTGTCTTTTCCCACGGCGACCTGGGAGACAGCAACATCTTTGT | |
| | GAAAGATGGCAAAGTAAGTGGCTTTATTGATCTTGGGAGAAGCGGCAGGGCGGACAAGTG | |
| | GTATGACATTGCCTTCTGCGTCCGGTCGATCAGGGAGGATATCGGGGAAGAACAGTATGT | |
| | CGAGCTATTTTTTGACTTACTGGGGATCAAGCCTGATTGGGAGAAAATAAAATATTATAT | |
| | TTTACTGGATGAATTGTTTTAGTCACATACAAATGGACGAACGGATAAACCTTTTCACGC | |
| | CCTTTTAAATATCCGATTATTCTAATAAACGCTCTTTTCTCTTAGGTTTACCCGCCAATA | |
| | TATCCTGTCAAACACTGATAGTTTAAACTGAAGGCGGGAAACGACAATCTGATCCTGGCG | |
| | AAAGGGGGATGTGCTGCAAGGCGATTAAGTTGGGTAACGCCAGGGTTTTCCCAGTCACGA | |
| | CGTTGTAAAACGACGGCCAGTGCCAAGCTTGCATGCCTGCAGGTCCCCAGATTAGCCTTT | |
| | TCAATTTCAGAAAGAATGCTAACCCACAGATGGTTAGAGAGGCTTACGCAGCAGGTCTCA | |
| | TCAAGACGATCTACCCGAGCAATAATCTCCAGGAAATCAAATACCTTCCCAAGAAGGTTA | |
| | AAGATGCAGTCAAAAGATTCAGGACTAACTGCATCAAGAACACAGAGAAAGATATATTTC | |
| | TCAAGATCAGAAGTACTATTCCAGTATGGACGATTCAAGGCTTGCTTCACAAACCAAGGC | |
| | AAGTAATAGAGATTGGAGTCTCTAAAAAGGTAGTTCCCACTGAATCAAAGGCCATGGAGT | |
| | CAAAGATTCAAATAGAGGACCTAACAGAACTCGCCGTAAAGACTGGCGAACAGTTCATAC | |
| | AGAGTCTCTTACGACTCAATGACAAGAAGAAAATCTTCGTCAACATGGTGGAGCACGACA | |
| | CACTTGTCTACTCCAAAAATATCAAAGATACAGTCTCAGAAGACCAAAGGGCAATTGAGA | |
| | CTTTTCAACAAAGGGTAATATCCGGAAACCTCCTCGGATTCCATTGCCCAGCTATCTGTC | |
| | ACTTTATTGTGAAGATAGTGGAAAAGGAAGGTGGCTCCTACAAATGCCATCATTGCGATA | |
| | AAGGAAAGGCCATCGTTGAAGATGCCTCTGCCGACAGTGGTCCCAAAGATGGACCCCCAC | |
| | CCACGAGGAGCATCGTGGAAAAAGAAGACGTTCCAACCACGTCTTCAAAGCAAGTGGATT | |
| | GATGTGATATCTCCACTGACGTAAGGGATGACGCACAATCCCACTATCCTTCGCAAGACC | |
| | CTTCCTCTATATAAGGAAGTTCATTTCATTTGGAGAGAACACGGGGGACTCTAGATACAT | |
| | CACAATCACACAAAACTAACAAAAGATCAAAAGCAAGTTCTTCACTGTTGATACATATGC | |
| | CCGTCGATTAGCAAGTTACTACCGGGTCGACAATTCTGAATCAACAACTCTCCTGGCGCA | |
| | CCATCGTCGGCTACAGCCTCGGGAATTGCTACCGAGCTCTTATGAAGATGAAGATGAAAT | |
| | ATTTGGTGTGTCAAATAAAAAGCTAGCTTGTGTGCTTAAGTTTGTGTTTTTTTCTTGGCT | |
| | TGTTGTGTTATGAATTTGTGGCTTTTTCTAATATTAAATGAATGTAAGATCTCATTATAA | |
| | TGAATAAACAAATGTTTCTATAATCCATTGTGAATGTTTTGTTGGATCTCTTCGCATATA | |
| | ACTACTGTATGTGCTATGGTATGGACTATGGAATATGATTAAAGATAAGGAATTGGTACC | |
| | TGCGGCCGCTGGATCCCGGGAATTCGTAATCATGGTCATAGCTGTTTCCTGTGTGAAATT | |
| | GTTATCCGCTCACAATTCCACACAACATACGAGCCGGAAGCATAAAGTGTAAAGCCTGGG | |
| | GTGCCTAATGAGTGAGCTAACTCACATTAATTGCGTTGCGCTCACTGCCCGCTTTCCAGT | |
| | CGGGAAACCTGTCGTGCCAGCTGCATTAATGAATCGGCCAACGCGCGGGGAGAGGCGGTT | |
| | TGCGTATTGGCTAGAGCAGCTTGCCAACATGGTGGAGCACGACACTCTCGTCTACTCCAA | |
| | GAATATCAAAGATACAGTCTCAGAAGACCAAAGGGCTATTGAGACTTTTCAACAAAGGGT | |
| | AATATCGGGAAACCTCCTCGGATTCCATTGCCCAGCTATCTGTCACTTCATCAAAAGGAC | |
| | AGTAGAAAAGGAAGGTGGCACCTACAAATGCCATCATTGCGATAAAGGAAAGGCTATCGT | |
| | TCAAGATGCCTCTGCCGACAGTGGTCCCAAAGATGGACCCCCACCCACGAGGAGCATCGT | |
| | GGAAAAAGAAGACGTTCCAACCACGTCTTCAAAGCAAGTGGATTGATGTGATATCTCCAC | |
| | TGACGTAAGGGATGACGCACAATCCCACTATCCTTCGCAAGACCTTCCTCTATATAAGGA | |
| | AGTTCATTTCATTTGGAGAGGACACGCTGGAATTCTAGTATACTAAACCATGAAAAAGCC | |
| | TGAACTCACCGCGACGTCTGTCGAGAAGTTTCTGATCGAAAAGTTCGACAGCGTCTCCGA | |
| | CCTGATGCAGCTCTCGGAGGGCGAAGAATCTCGTGCTTTCAGCTTCGATGTAGGAGGGCG | |
| | TGGATATGTCCTGCGGGTAAATAGCTGCGCCGATGGTTTCTACAAAGATCGTTATGTTTA | |
| | TCGGCACTTTGCATCGGCCGCGCTCCCGATTCCGGAAGTGCTTGACATTGGGGAATTCAG | |
| | CGAGAGCCTGACCTATTGCATCTCCCGCCGTGCACAGGGTGTCACGTTGCAAGACCTGCC | |
| | TGAAACCGAACTGCCCGCTGTTCTGCAGCCGGTCGCGGAGGCCATGGATGCGATCGCTGC | |
| | GGCCGATCTTAGCCAGACGAGCGGGTTCGGCCCATTCGGACCGCAAGGAATCGGTCAATA | |
| | CACTACATGGCGTGATTTCATATGCGCGATTGCTGATCCCCATGTGTATCACTGGCAAAC | |
| | TGTGATGGACGACACCGTCAGTGCGTCCGTCGCGCAGGCTCTCGATGAGCTGATGCTTTG | |
| | GGCCGAGGACTGCCCCGAAGTCCGGCACCTCGTGCACGCGGATTTCGGCTCCAACAATGT | |
| | CCTGACGGACAATGGCCGCATAACAGCGGTCATTGACTGGAGCGAGGCGATGTTCGGGGA | |
| | TTCCCAATACGAGGTCGCCAACATCTTCTTCTGGAGGCCGTGGTTGGCTTGTATGGAGCA | |
| | GCAGACGCGCTACTTCGAGCGGAGGCATCCGGAGCTTGCAGGATCGCCGCGGCTCCGGGC | |
| | GTATATGCTCCGCATTGGTCTTGACCAACTCTATCAGAGCTTGGTTGACGGCAATTTCGA | |
| | TGATGCAGCTTGGGCGCAGGGTCGATGCGACGCAATCGTCCGATCCGGAGCCGGGACTGT | |
| | CGGGCGTACACAAATCGCCCGCAGAAGCGCGGCCGTCTGGACCGATGGCTGTGTAGAAGT | |
| | ACTCGCCGATAGTGGAAACCGACGCCCCAGCACTCGTCCGAGGGCAAAGGAATAGGCGGG | |
| | ACTCTGGGGTTCGAAATGACCGACCAAGCGACGCCCAACCTGCCATCACGAGATTTCGAT | |
| | TCCACCGCCGCCTTCTATGAAAGGTTGGGCTTCGGAATCGTTTTCCGGGACGCCGGCTGG | |
| | ATGATCCTCCAGCGCGGGGATCTCATGCTGGAGTTCTTCGCCCACGGGATCTCTGCGGAA | |
| | CAGGCGGTCGAAGGTGCCGATATCATTACGACAGCAACGGCCGACAAGCACAACGCCACG | |
| | ATCCTGAGCGACAATATGATCGGGCCCCGATCGTTCAAACATTTGGCAATAAAGTTTCTT | |
| | AAGATTGAATCCTGTTGCCGGTCTTGCGATGATTATCATATAATTTCTGTTGAATTACGT | |
| | TAAGCATGTAATAATTAACATGTAATGCATGACGTTATTTATGAGATGGGTTTTTATGAT | |
| | TAGAGTCCCGCAATTATACATTTAATACGCGATAGAAAACAAAATATAGCGCGCAAACTA | |
| | GGATAAATTATCGCGCGCGGTGTCATCTATGTTACTAGATCGGGAATTTGGGCCATCGCC | |
| | CTGATAGACGGTTTTTCGCCCTTTGACGTTGGAGTCCACGTTCTTTAATAGTGGACTCTT | |
| | GTTCCAAACTGGAACAACACTCAACCCTATCTCGGGCTATTCTTTTGATTTATAAGGGAT | |
| | TTTGCCGATTTCGGAACCACCATCAAACAGGATTTTCGCCTGCTGGGGCAAACCAGCGTG | |
| | GACCGCTTGCTGCAACTCTCTCAGGGCCAGGCGGTGAAGGGCAATCAGCTGTTGCCCGTC | |
| | TCACTGGTGAAAAGAAAAACCACCCCAGTACATTAAAAACGTCCGCAATGTGTTATTAAG | |
| | TTGTCTAAGCGTCAATTTGTTTACACCACAATATATCCTGCCACAAGCTAGCTTTCTGAG | |
| | CCGCCGATTTTCCTCCTCGAGTTGGATGAACTCGCCGAGTTCATCGTCAACTGAAACAGA | |
| | CACGGCCGGATTCTGTGAGACAGGTTGAACCGCAGCTCTCTTCCATTGATAATAGGTCTG | |
| | AACGGAAATACCCACGATCTTAACGGCGTCCTTCAAGGTTGCGCCGCCAGCGACCTGAGC | |
| | TTCGATTTGACCGATCTTCTCCAGTTTTTCTCGGTTGCTGAGGCCGCGGGTTTTCGGCTT | |
| | CACGGATTTGAACGATCCCGTGCGGGCTGTTTCGGCTGGTGCTTTCTTTGCTCTTCTACC | |
| | TCTAGGAGCAGCCGGCTCAACTTCGGCAGCAGCAGTACCGTCCGGCGGATTCTGGATCTC | |
| | TTCGTCAGCCATTAATCGTCCTCTGTGTGGGTTATTGCTTTGTCTGCCAGCTCGATCCAA | |
| | GAGTCAACGTTTGTGCCTAGGGCAGTAAATAGGCAGTGCTCCGCGACTACATGCCTCGGC | |
| | CGGCAAAATACCGCCGCATGTAGAGCAGGCTCTCCTTCACGATCAACGATCGGCATGGGG | |
| | CCTTCGTGCTTGTTGAGTAATGTTATCGCTCCCATCAGAGCACGCTTGGTACTCCGGGAA | |
| | TCGGATGGTCTGTCGATCATCCAAAAAACGCTCATGTTTTCAACCTATTAGGTCTGTGGT | |
| | CAGCTGACCACAGACCATCCTGCTCCATACTCGCTAATTCTAGCCAAACCGCAACGTCCC | |
| | CTGCCCGCTAGCCTTCAAGAGCGCCATTATCATCGGGCCAAGTGAAAACTTCCCGAGTTC | |
| | GCTCCGCCGTGTCAGATCTCGGAGATAGCCCCCAGGCGAATTGATGAAGTTCGCTCGCTC | |
| | CAAAATGCACGCCATCGCTGCTGCCGCATTCTCCGGTCCCATTGCCTCACACGCGTCTTG | |
| | GTAAGCCGACGGGCTGACCCCCAGCATAGACCGAACCACCACCGCAGCCGACATGAGGTC | |
| | ACGCCAGCTAGCAACCGCACCGCTCGGCCCATAATTGCCAATGGTCGGGCACGCTTTCAG | |
| | GATCATCCCGAGGGGGAACGCTTTTATCGGCTCGCTCCTTGCCCGGTCTATTTCACTCGG | |
| | CTTAGCGCCCTGCTCCTTTTCAGAGCGAGGTTCAAGTTCATTAACGGATTCGGGTTTTGA | |
| | GTTCTGTATGTGCTGCTCGCTCTGGGCAGCATTGGTGCTATTATTTTCTGAATTGTCTCT | |
| | AATTTCCAACCGGTTGATTATCTCTTCCTGGAGCATCCACATCTCTTCGAGAATTGACTC | |
| | TACATCAGCAAGCGTCGGGGCGCGTGGGATTCTACCCACAAGTTCCACATAGACTTCCTC | |
| | GACAGCTTGCCAGTCGCCCTCCGCTCCCTCTTCCATAGCTGCCGTAATTAGCTTCCGAAC | |
| | GTCCCGTCGGCAAATCGTCAGACTTTCTTTGGCCATCCTGAATGCTGCTCGATCGGCCAT | |
| | CACCTGCTGTGCCATCATCGCTAGCTCTTCGGACCGCGCGAGAAGCGGAGACAAATCGAA | |
| | GCCAAACGCGCGCTCGATCTGACCAGCGCCATCCTTACGAGCGTAACGCTTTCCGTTGGC | |
| | GCTATCCTTCCGGACGATCAAGCCTGACTCCACGAGCATGGCGATGTGCCTACGCAAAGT | |
| | CGCGCCAGCCATCCCATGCGCCCGAAGGGCAAGCTGAGCATTCGACGGGAAGACGATCAG | |
| | CTGTGCCTCCTGACGCAACTCCGTTTCCGGGTGAAAGCTCAATAGCGCATCAAGGACGGC | |
| | AAGACTGTTGGACTGGATTCCAAGTAGTTCCATGGCCGCGGACGCGTCCCTAAAGACCTT | |
| | CCACTTGTCCGCTGTCTTGCCTTGTTTGATATCGGCCAGCGCCGTCTGGCGCCGCACAAG | |
| | CGCAAGCGTCATTGGCCGCCGCCCGAATGGCGTCGTTACACTTCCTGTCTGCATCATCTT | |
| | TCACCTTTCAGCAGGCAAAGGAAATCAGCTCACCAAAACGGCGCTAAAAACTCTTGACGA | |
| | GGATTCGAGGAAATGCGATTCTGTTCGCGCTAGAGAGACAGAAGGGCTTCCGCGACGGCG | |
| | ACGTTGAGGGGGCTCTTTTCTTTTGCGGTTTACTCTCCCCGTTTCCGTTGGTTCTCAGCG | |
| | TGGTACGCTTGATACAGCGCTGGCACATGATCGAGCACGAAGGTCGCAAAATCGGGCGTC | |
| | GCCTTCCTGTCAATCGTGATTTCCAGTTTGGCCTTGCTCTGCGTCACCTGTGCAATTCTG | |
| | GTGCCGTCTGGGGTGGCCATGACCTCGGGAAGTCCACGCGCAACCCGACTGGGCTTCAGA | |
| | CTAGCGATCACCGCCTTGAATCGTTCTGCCGATGGCAGCGCTTGAACTTCCTCCGACATA | |
| | GCATATTTAGCCACGTCGGCCGGTGAAGAAACTTTCTCAATCAGCTCGGCAAGTTGTTGC | |
| | CAACTCGGCCGTCCAACACCAGGAGCGGCACCAATAGCATCGGTCAGTTCAGAGGGGAGG | |
| | GCGTCAACGAGCAGAAGCATCTTGGACAAATTGCTCTTGTCGATCGACATCGCGGCGATG | |
| | ACAATCTCTCGAGAAAACTGCCTGTTCAGGCGATGTGCGAAGCGCGCCTTTTCGATGAAG | |
| | GTAAGATCTTCGCGCACATTGTTTTCCTGACCCTGTGCTACGACCACTTGCTCGTCCGTC | |
| | AGTTCGCGAACGACCGCCCTGACCGGAAGTCCGAGTTCTGAAACGGCGCGTAGCCGGCGG | |
| | TGGCCGAAGGCAACCTGATATCGGCCCGGCTGGCTCGGATGCGGTCGCACAAGGATTGGG | |
| | ACTTGCTGTCCTTGTTCCCGGATCGAAGTAAGGAGCCCGTCAATGTCCCCTCGCATACGA | |
| | TCCTGCACGAAAGACGGTTCTATTGACGAGGCATCCAACTCTATCACTGCCTGACCTTCA | |
| | GCGAGACGCCGCTCGATCTCTTCGGCACGGCTAAGACGATCGTTTTGCTCTCGCAGTGCG | |
| | TTACCAATGTTCGCTGTGAGCTTCGTTGCCGGATCGCGCTCCTTCCTTGTTACGCCGAGG | |
| | AGCGGCATGGAGCGGTTCTTTGCCGTCCTATTGTCGGCGGGCGACGTCTCAGGGGCGTCA | |
| | GTTGAGACGCCAAGGATGTGCTTCCGGCTCATGTGGGCCTACCCCATGCTTTTTTGATCA | |
| | GTGTTTCGATCTCGTCGTTGACGGCGTTCATCGCCTCCAAGGCTCGATCATAGGTCGAGC | |
| | GCGTGAACAGGCCACGCTCCACTTCGAATAGAGTCTGGTTTGTCAGGCCAGCGTCCGAAA | |
| | CCGCGGTGGTTTTAAGCATCGGAAAATTGAGGACATTTTCGCCAAAAATCGACCGCAGAT | |
| | AACCTACCATTTGGTTCTGTGGTCCGTCGCTCGGTTCGAAACGGGTTATCAGATAGCGCA | |
| | TCCAATTAAACTTGAACTTGGCGCCAGCATTCTCGATTTCACGCAAAAGGTTCGATGTCA | |
| | TTGCCAGAAACTGGTTCATCGACATCACATCCAGCATCTGCGGATGGACCGTGACAAGAA | |
| | TGGACGTCGCCGCAGTCAATGCGGATAGCGTGAGATACCCAAGCTGGGGAGGGCAGTCGA | |
| | TGACCACGACGTCATAGTTATCCGCGATATCTTCAATTACTTGGCTGATGCGACCATAAA | |
| | AGAGCGTGTCGCCCTCTTTGCGGTTCATCAGCGCGCGTGGCGTATCGTGTTCAAACTCCA | |
| | TCAGCTCAAGGTTACCAGGAATCAGGTGGAGGTCGGGAATGTAAGTCCCTCGGACGACTC | |
| | GTTCGATTGCCACCTGCTCATCATCATACCTTATAGCGCCGTAGAGCGTTTCGTTCGGGC | |
| | CAACGTCCGTCTCCGGTTGGCTCCCAAAGAGTGCAGAAAGGCTCGCTTGAGGATCGAGAT | |
| | CAATGGCCAAGACTCGATATCCGCGCATAGCGAGGTACTGCGCCAGATGCGCGGCGGTGG | |
| | TGGTCTTACCCGACCCACCTTTGAAATTCATCACAGAGATAACCTGAAGCTGCTCGCCGC | |
| | CTCGACGATGTGGCAGGTAGCGCCGGTTCCCGCGGCCGACCTGATCCATATACTTCCGAA | |
| | TCACATGGATATCTTCAATTGAGAACATTCGCCTGCCACCTGGGCTCATGCTAACATTCA | |
| | ACTCTGGCATCTCAGACGCGGTCTGCCGTAAATATGACTCGCCAACGCCGAGCAGCTTGG | |
| | ACGCCTCCGATGGCCCGAATGTTCGAATACCCTTCTCGGAATGCGGCGGGAAAACCTTAA | |
| | GATGATGTGCTTGAAGTTGGCTCGAGAGGGCATCGGCATGACGCTCCATCAAGGCCGTCA | |
| | ACCCTACAACTACAGGCGCTGCTTTTAGGACAGACTTCGCCATCTCAAACCCATTCCTTG | |
| | CCAGTGGCGATATTTTTCGCGAAACTGGAAAAGTTCCGCCGCTGGCAATTAGCGCCGATT | |
| | CTGCTGTTTGGGCAAGAGCTTTTAGGTTAACAGAAGGTTAACGCCCTCAGGTCGAAAAAC | |
| | TCCACCCAACTGTTATTTGTATTTATTTCCAATGCCTTAGAGAGATTGCCATTTGAATAT | |
| | GTTCATGTATTGTTTTAGTGATAATCCTACAATCGTAACCCAAAAAGAGGTCGCCCTCTG | |
| | CGCGCCGTCGTCCAATATAGGCGAAGTCACCCTTGCGACTCAGGCGGATTCTACCTTGTA | |
| | GGATCGAGTCAGGCAACTATGGATGAACGAAATAGACAGATCGCTGAGATAGGTGCCTCA | |
| | CTGATTAAGCATTGGTAACTGTCAGACCAAGTTTACTCATATATACTTTAGATTGATTTA | |
| | AAACTTCATTTTTAATTTAAAAGGATCTAGGTGAAGATCCTTTTTGATAATCTCATGACC | |
| | AAAATCCCTTAACGTGAGTTTTCGTTCCACTGAGCGTCAGACCCCGTAGAAAAGATCAAA | |
| | GGATCTTCTTGAGATCCTTTTTTTCTGCGCGTAATCTGCTGCTTGCAAACAAAAAAACCA | |
| | CCGCTACCAGCGGTGGTTTGTTTGCCGGATCAAGAGCTACCAACTCTTTTTCCGAAGGTA | |
| | ACTGGCTTCAGCAGAGCGCAGATACCAAATACTGTTCTTCTAGTGTAGCCGTAGTTAGGC | |
| | CACCACTTCAAGAACTCTGTAGCACCGCCTACATACCTCGCTCTGCTAATCCTGTTACCA | |
| | GTGGCTGCTGCCAGTGGCGATAAGTCGTGTCTTACCGGGTTGGACTCAAGACGATAGTTA | |
| | CCGGATAAGGCGCAGCGGTCGGGCTGAACGGGGGGTTCGTGCACACAGCCCAGCTTGGAG | |
| | CGAACGACCTACACCGAACTGAGATACCTACAGCGTGAGCTATGAGAAAGCGCCACGCTT | |
| | CCCGAAGGGAGAAAGGCGGACAGGTATCCGGTAAGCGGCAGGGTCGGAACAGGAGAGCGC | |
| | ACGAGGGAGCTTCCAGGGGGAAACGCCTGGTATCTTTATAGTCCTGTCGGGTTTCGCCAC | |
| | CTCTGACTTGAGCGTCGATTTTTGTGATGCTCGTCAGGGGGGCGGAGCCTATGGAAAAAC | |
| | GCCAGCAACGCGGCCTTTTTACGGTTCCTGGCCTTTTGCTGGCCTTTTGCTCA | |
| 35S::hph | CAACATGGTGGAGCACGACACTCTCGTCTACTCCAAGAATATCAAAGATACAGTCTCAGA | 44 |
| cassette | AGACCAAAGGGCTATTGAGACTTTTCAACAAAGGGTAATATCGGGAAACCTCCTCGGATT | |
| | CCATTGCCCAGCTATCTGTCACTTCATCAAAAGGACAGTAGAAAAGGAAGGTGGCACCTA | |
| | CAAATGCCATCATTGCGATAAAGGAAAGGCTATCGTTCAAGATGCCTCTGCCGACAGTGG | |
| | TCCCAAAGATGGACCCCCACCCACGAGGAGCATCGTGGAAAAAGAAGACGTTCCAACCAC | |
| | GTCTTCAAAGCAAGTGGATTGATGTGATATCTCCACTGACGTAAGGGATGACGCACAATC | |
| | CCACTATCCTTCGCAAGACCTTCCTCTATATAAGGAAGTTCATTTCATTTGGAGAGGACA | |
| | CGCTGGAATTCTAGTATACTAAACCATGAAAAAGCCTGAACTCACCGCGACGTCTGTCGA | |
| | GAAGTTTCTGATCGAAAAGTTCGACAGCGTCTCCGACCTGATGCAGCTCTCGGAGGGCGA | |
| | AGAATCTCGTGCTTTCAGCTTCGATGTAGGAGGGCGTGGATATGTCCTGCGGGTAAATAG | |
| | CTGCGCCGATGGTTTCTACAAAGATCGTTATGTTTATCGGCACTTTGCATCGGCCGCGCT | |
| | CCCGATTCCGGAAGTGCTTGACATTGGGGAATTCAGCGAGAGCCTGACCTATTGCATCTC | |
| | CCGCCGTGCACAGGGTGTCACGTTGCAAGACCTGCCTGAAACCGAACTGCCCGCTGTTCT | |
| | GCAGCCGGTCGCGGAGGCCATGGATGCGATCGCTGCGGCCGATCTTAGCCAGACGAGCGG | |
| | GTTCGGCCCATTCGGACCGCAAGGAATCGGTCAATACACTACATGGCGTGATTTCATATG | |
| | CGCGATTGCTGATCCCCATGTGTATCACTGGCAAACTGTGATGGACGACACCGTCAGTGC | |
| | GTCCGTCGCGCAGGCTCTCGATGAGCTGATGCTTTGGGCCGAGGACTGCCCCGAAGTCCG | |
| | GCACCTCGTGCACGCGGATTTCGGCTCCAACAATGTCCTGACGGACAATGGCCGCATAAC | |
| | AGCGGTCATTGACTGGAGCGAGGCGATGTTCGGGGATTCCCAATACGAGGTCGCCAACAT | |
| | CTTCTTCTGGAGGCCGTGGTTGGCTTGTATGGAGCAGCAGACGCGCTACTTCGAGCGGAG | |
| | GCATCCGGAGCTTGCAGGATCGCCGCGGCTCCGGGCGTATATGCTCCGCATTGGTCTTGA | |
| | CCAACTCTATCAGAGCTTGGTTGACGGCAATTTCGATGATGCAGCTTGGGCGCAGGGTCG | |
| | ATGCGACGCAATCGTCCGATCCGGAGCCGGGACTGTCGGGCGTACACAAATCGCCCGCAG | |
| | AAGCGCGGCCGTCTGGACCGATGGCTGTGTAGAAGTACTCGCCGATAGTGGAAACCGACG | |
| | CCCCAGCACTCGTCCGAGGGCAAAGGAATAG | |
| pRI-Cas9- | GTTGCCATGTTTTACGGCAGTGAGAGCAGAGATAGCGCTGATGTCCGGCGGTGCTTTTGC | 45 |
| HygroR | CGTTACGCACCACCCCGTCAGTAGCTGAACAGGAGGGACAGCTGATAGAAACAGAAGCCA | |
| | CTGGAGCACCTCAAAAACACCATCATACACTAAATCAGTAAGTTGGCAGCATCACCCATA | |
| | ATTGTGGTTTCAAAATCGGCTCCGTCGATACTATGTTATACGCCAACTTTGAAAACAACT | |
| | TTGAAAAAGCTGTTTTCTGGTATTTAAGGTTTTAGAATGCAAGGAACAGTGAATTGGAGT | |
| | TCGTCTTGTTATAATTAGCTTCTTGGGGTATCTTTAAATACTGTAGAAAAGAGGAAGGAA | |
| | ATAATAAATGGCTAAAATGAGAATATCACCGGAATTGAAAAAACTGATCGAAAAATACCG | |
| | CTGCGTAAAAGATACGGAAGGAATGTCTCCTGCTAAGGTATATAAGCTGGTGGGAGAAAA | |
| | TGAAAACCTATATTTAAAAATGACGGACAGCCGGTATAAAGGGACCACCTATGATGTGGA | |
| | ACGGGAAAAGGACATGATGCTATGGCTGGAAGGAAAGCTGCCTGTTCCAAAGGTCCTGCA | |
| | CTTTGAACGGCATGATGGCTGGAGCAATCTGCTCATGAGTGAGGCCGATGGCGTCCTTTG | |
| | CTCGGAAGAGTATGAAGATGAACAAAGCCCTGAAAAGATTATCGAGCTGTATGCGGAGTG | |
| | CATCAGGCTCTTTCACTCCATCGACATATCGGATTGTCCCTATACGAATAGCTTAGACAG | |
| | CCGCTTAGCCGAATTGGATTACTTACTGAATAACGATCTGGCCGATGTGGATTGCGAAAA | |
| | CTGGGAAGAAGACACTCCATTTAAAGATCCGCGCGAGCTGTATGATTTTTTAAAGACGGA | |
| | AAAGCCCGAAGAGGAACTTGTCTTTTCCCACGGCGACCTGGGAGACAGCAACATCTTTGT | |
| | GAAAGATGGCAAAGTAAGTGGCTTTATTGATCTTGGGAGAAGCGGCAGGGCGGACAAGTG | |
| | GTATGACATTGCCTTCTGCGTCCGGTCGATCAGGGAGGATATCGGGGAAGAACAGTATGT | |
| | CGAGCTATTTTTTGACTTACTGGGGATCAAGCCTGATTGGGAGAAAATAAAATATTATAT | |
| | TTTACTGGATGAATTGTTTTAGTCACATACAAATGGACGAACGGATAAACCTTTTCACGC | |
| | CCTTTTAAATATCCGATTATTCTAATAAACGCTCTTTTCTCTTAGGTTTACCCGCCAATA | |
| | TATCCTGTCAAACACTGATAGTTTAAACTGAAGGCGGGAAACGACAATCTGATCCTGGCG | |
| | AAAGGGGGATGTGCTGCAAGGCGATTAAGTTGGGTAACGCCAGGGTTTTCCCAGTCACGA | |
| | CGTTGTAAAACGACGGCCAGTGCCAAGCTTGCATGCCTGCAGGTCCCCAGATTAGCCTTT | |
| | TCAATTTCAGAAAGAATGCTAACCCACAGATGGTTAGAGAGGCTTACGCAGCAGGTCTCA | |
| | TCAAGACGATCTACCCGAGCAATAATCTCCAGGAAATCAAATACCTTCCCAAGAAGGTTA | |
| | AAGATGCAGTCAAAAGATTCAGGACTAACTGCATCAAGAACACAGAGAAAGATATATTTC | |
| | TCAAGATCAGAAGTACTATTCCAGTATGGACGATTCAAGGCTTGCTTCACAAACCAAGGC | |
| | AAGTAATAGAGATTGGAGTCTCTAAAAAGGTAGTTCCCACTGAATCAAAGGCCATGGAGT | |
| | CAAAGATTCAAATAGAGGACCTAACAGAACTCGCCGTAAAGACTGGCGAACAGTTCATAC | |
| | AGAGTCTCTTACGACTCAATGACAAGAAGAAAATCTTCGTCAACATGGTGGAGCACGACA | |
| | CACTTGTCTACTCCAAAAATATCAAAGATACAGTCTCAGAAGACCAAAGGGCAATTGAGA | |
| | CTTTTCAACAAAGGGTAATATCCGGAAACCTCCTCGGATTCCATTGCCCAGCTATCTGTC | |
| | ACTTTATTGTGAAGATAGTGGAAAAGGAAGGTGGCTCCTACAAATGCCATCATTGCGATA | |
| | AAGGAAAGGCCATCGTTGAAGATGCCTCTGCCGACAGTGGTCCCAAAGATGGACCCCCAC | |
| | CCACGAGGAGCATCGTGGAAAAAGAAGACGTTCCAACCACGTCTTCAAAGCAAGTGGATT | |
| | GATGTGATATCTCCACTGACGTAAGGGATGACGCACAATCCCACTATCCTTCGCAAGACC | |
| | CTTCCTCTATATAAGGAAGTTCATTTCATTTGGAGAGAACACGGGGGACTCTAGATACAT | |
| | CACAATCACACAAAACTAACAAAAGATCAAAAGCAAGTTCTTCACTGTTGATACATatgg | |
| | ataagaagtactctatcggactcgatatcggaactaactctgtgggatgggctgtgatca | |
| | ccgatgagtacaaggtgccatctaagaagttcaaggttctcggaaacaccgataggcact | |
| | ctatcaagaaaaaccttatcggtgctctcctcttcgattctggtgaaactgctgaggcta | |
| | ccagactcaagagaaccgctagaagaaggtacaccagaagaaagaacaggatctgctacc | |
| | tccaagagatcttctctaacgagatggctaaagtggatgattcattcttccacaggctcg | |
| | aagagtcattcctcgtggaagaagataagaagcacgagaggcaccctatcttcggaaaca | |
| | tcgttgatgaggtggcataccacgagaagtaccctactatctaccacctcagaaagaagc | |
| | tcgttgattctactgataaggctgatctcaggctcatctacctcgctctcgctcacatga | |
| | tcaagttcagaggacacttcctcatcgagggtgatctcaaccctgataactctgatgtgg | |
| | ataagttgttcatccagctcgtgcagacctacaaccagcttttcgaagagaaccctatca | |
| | acgcttcaggtgtggatgctaaggctatcctctctgctaggctctctaagtcaagaaggc | |
| | ttgagaacctcattgctcagctccctggtgagaagaagaacggacttttcggaaacttga | |
| | tcgctctctctctcggactcacccctaacttcaagtctaacttcgatctcgctgaagatg | |
| | caaagctccagctctcaaaggatacctacgatgatgatctcgataacctcctcgctcaga | |
| | tcggagatcagtacgctgatttgttcctcgctgctaagaacctctctgatgctatcctcc | |
| | tcagtgatatcctcagagtgaacaccgagatcaccaaggctccactctcagcttctatga | |
| | tcaagagatacgatgagcaccaccaggatctcacacttctcaaggctcttgttagacagc | |
| | agctcccagagaagtacaaagagattttcttcgatcagtctaagaacggatacgctggtt | |
| | acatcgatggtggtgcatctcaagaagagttctacaagttcatcaagcctatcctcgaga | |
| | agatggatggaaccgaggaactcctcgtgaagctcaatagagaggatcttctcagaaagc | |
| | agaggaccttcgataacggatctatccctcatcagatccacctcggagagttgcacgcta | |
| | tccttagaaggcaagaggatttctacccattcctcaaggataacagggaaaagattgaga | |
| | agattctcaccttcagaatcccttactacgtgggacctctcgctagaggaaactcaagat | |
| | tcgcttggatgaccagaaagtctgaggaaaccatcaccccttggaacttcgaagaggtgg | |
| | tggataagggtgctagtgctcagtctttcatcgagaggatgaccaacttcgataagaacc | |
| | ttccaaacgagaaggtgctccctaagcactctttgctctacgagtacttcaccgtgtaca | |
| | acgagttgaccaaggttaagtacgtgaccgagggaatgaggaagcctgcttttttgtcag | |
| | gtgagcaaaagaaggctatcgttgatctcttgttcaagaccaacagaaaggtgaccgtga | |
| | agcagctcaaagaggattacttcaagaaaatcgagtgcttcgattcagttgagatttctg | |
| | gtgttgaggataggttcaacgcatctctcggaacctaccacgatctcctcaagatcatta | |
| | aggataaggatttcttggataacgaggaaaacgaggatatcttggaggatatcgttctta | |
| | ccctcaccctctttgaagatagagagatgattgaagaaaggctcaagacctacgctcatc | |
| | tcttcgatgataaggtgatgaagcagttgaagagaagaagatacactggttggggaaggc | |
| | tctcaagaaagctcattaacggaatcagggataagcagtctggaaagacaatccttgatt | |
| | tcctcaagtctgatggattcgctaacagaaacttcatgcagctcatccacgatgattctc | |
| | tcacctttaaagaggatatccagaaggctcaggtttcaggacagggtgatagtctccatg | |
| | agcatatcgctaacctcgctggatctcctgcaatcaagaagggaatcctccagactgtga | |
| | aggttgtggatgagttggtgaaggtgatgggaaggcataagcctgagaacatcgtgatcg | |
| | aaatggctagagagaaccagaccactcagaagggacagaagaactctagggaaaggatga | |
| | agaggatcgaggaaggtatcaaagagcttggatctcagatcctcaaagagcaccctgttg | |
| | agaacactcagctccagaatgagaagctctacctctactacctccagaacggaagggata | |
| | tgtatgtggatcaagagttggatatcaacaggctctctgattacgatgttgatcatatcg | |
| | tgccacagtcattcttgaaggatgattctatcgataacaaggtgctcaccaggtctgata | |
| | agaacaggggtaagagtgataacgtgccaagtgaagaggttgtgaagaaaatgaagaact | |
| | attggaggcagctcctcaacgctaagctcatcactcagagaaagttcgataacttgacta | |
| | aggctgagaggggaggactctctgaattggataaggcaggattcatcaagaggcagcttg | |
| | tggaaaccaggcagatcactaagcacgttgcacagatcctcgattctaggatgaacacca | |
| | agtacgatgagaacgataagttgatcagggaagtgaaggttatcaccctcaagtcaaagc | |
| | tcgtgtctgatttcagaaaggatttccaattctacaaggtgagggaaatcaacaactacc | |
| | accacgctcacgatgcttaccttaacgctgttgttggaaccgctctcatcaagaagtatc | |
| | ctaagctcgagtcagagttcgtgtacggtgattacaaggtgtacgatgtgaggaagatga | |
| | tcgctaagtctgagcaagagatcggaaaggctaccgctaagtatttcttctactctaaca | |
| | tcatgaatttcttcaagaccgagattaccctcgctaacggtgagatcagaaagaggccac | |
| | tcatcgagacaaacggtgaaacaggtgagatcgtgtgggataagggaagggatttcgcta | |
| | ccgttagaaaggtgctctctatgccacaggtgaacatcgttaagaaaaccgaggtgcaga | |
| | ccggtggattctctaaagagtctatcctccctaagaggaactctgataagctcattgcta | |
| | ggaagaaggattgggaccctaagaaatacggtggtttcgattctcctaccgtggcttact | |
| | ctgttctcgttgtggctaaggttgagaagggaaagagtaagaagctcaagtctgttaagg | |
| | aacttctcggaatcactatcatggaaaggtcatctttcgagaagaacccaatcgatttcc | |
| | tcgaggctaagggatacaaagaggttaagaaggatctcatcatcaagctcccaaagtact | |
| | cactcttcgaactcgagaacggtagaaagaggatgctcgcttctgctggtgagcttcaaa | |
| | agggaaacgagcttgctctcccatctaagtacgttaactttctttacctcgcttctcact | |
| | acgagaagttgaagggatctccagaagataacgagcagaagcaacttttcgttgagcagc | |
| | acaagcactacttggatgagatcatcgagcagatctctgagttctctaaaagggtgatcc | |
| | tcgctgatgcaaacctcgataaggtgttgtctgcttacaacaagcacagagataagccta | |
| | tcagggaacaggcagagaacatcatccatctcttcacccttaccaacctcggtgctcctg | |
| | ctgctttcaagtacttcgatacaaccatcgataggaagagatacacctctaccaaagaag | |
| | tgctcgatgctaccctcatccatcagtctatcactggactctacgagactaggatcgatc | |
| | tctcacagctaggtggtgattcaagggctgatcctaagaagaagaggaaggtttgaGTCG | |
| | ACAATTCTGAATCAACAACTCTCCTGGCGCACCATCGTCGGCTACAGCCTCGGGAATTGC | |
| | TACCGAGCTCTTATGAAGATGAAGATGAAATATTTGGTGTGTCAAATAAAAAGCTAGCTT | |
| | GTGTGCTTAAGTTTGTGTTTTTTTCTTGGCTTGTTGTGTTATGAATTTGTGGCTTTTTCT | |
| | AATATTAAATGAATGTAAGATCTCATTATAATGAATAAACAAATGTTTCTATAATCCATT | |
| | GTGAATGTTTTGTTGGATCTCTTCGCATATAACTACTGTATGTGCTATGGTATGGACTAT | |
| | GGAATATGATTAAAGATAAGGAATTGGTACCTGCGGCTTTTTTTCTTCTTCTTCGTTCAT | |
| | ACAGTTTTTTTTTGTTTATCAGCTTACATTTTCTTGAACCGTAGCTTTCGTTTTCTTCTT | |
| | TTTAACTTTCCATTCGGAGTTTTTGTATCTTGTTTCATAGTTTGTCCCAGGATTAGAATG | |
| | ATTAGGCATCGAACCTTCAAGAATTTGATTGAATAAAACATCTTCATTCTTAAGATATGA | |
| | AGATAATCTTCAAAAGGCCCCTGGGAATCTGAAAGAAGAGAAGCAGGCCCATTTATATGG | |
| | GAAAGAACAATAGTATTTCTTATATAGGCCCATTTAAGTTGAAAACAATCTTCAAAAGTC | |
| | CCACATCGCTTAGATAAGAAAACGAAGCTGAGTTTATATACAGCTAGAGTCGAAGTAGTG | |
| | ATTGnnnnnnnnnnnnnnnnnnnnGTTTTAGAGCTAGAAATAGCAAGTTAAAATAAGGCT | |
| | AGTCCGTTATCAACTTGAAAAAGTGGCACCGAGTCGGTGCTTTTTTTCTAGACCCAGCTT | |
| | TCTTGTACAAAGTTGGCATTACGCTGGATCCCGGGAATTCGTAATCATGGTCATAGCTGT | |
| | TTCCTGTGTGAAATTGTTATCCGCTCACAATTCCACACAACATACGAGCCGGAAGCATAA | |
| | AGTGTAAAGCCTGGGGTGCCTAATGAGTGAGCTAACTCACATTAATTGCGTTGCGCTCAC | |
| | TGCCCGCTTTCCAGTCGGGAAACCTGTCGTGCCAGCTGCATTAATGAATCGGCCAACGCG | |
| | CGGGGAGAGGCGGTTTGCGTATTGGCTAGAGCAGCTTGCCAACATGGTGGAGCACGACAC | |
| | TCTCGTCTACTCCAAGAATATCAAAGATACAGTCTCAGAAGACCAAAGGGCTATTGAGAC | |
| | TTTTCAACAAAGGGTAATATCGGGAAACCTCCTCGGATTCCATTGCCCAGCTATCTGTCA | |
| | CTTCATCAAAAGGACAGTAGAAAAGGAAGGTGGCACCTACAAATGCCATCATTGCGATAA | |
| | AGGAAAGGCTATCGTTCAAGATGCCTCTGCCGACAGTGGTCCCAAAGATGGACCCCCACC | |
| | CACGAGGAGCATCGTGGAAAAAGAAGACGTTCCAACCACGTCTTCAAAGCAAGTGGATTG | |
| | ATGTGATATCTCCACTGACGTAAGGGATGACGCACAATCCCACTATCCTTCGCAAGACCT | |
| | TCCTCTATATAAGGAAGTTCATTTCATTTGGAGAGGACACGCTGGAATTCTAGTATACTA | |
| | AACCATGAAAAAGCCTGAACTCACCGCGACGTCTGTCGAGAAGTTTCTGATCGAAAAGTT | |
| | CGACAGCGTCTCCGACCTGATGCAGCTCTCGGAGGGCGAAGAATCTCGTGCTTTCAGCTT | |
| | CGATGTAGGAGGGCGTGGATATGTCCTGCGGGTAAATAGCTGCGCCGATGGTTTCTACAA | |
| | AGATCGTTATGTTTATCGGCACTTTGCATCGGCCGCGCTCCCGATTCCGGAAGTGCTTGA | |
| | CATTGGGGAATTCAGCGAGAGCCTGACCTATTGCATCTCCCGCCGTGCACAGGGTGTCAC | |
| | GTTGCAAGACCTGCCTGAAACCGAACTGCCCGCTGTTCTGCAGCCGGTCGCGGAGGCCAT | |
| | GGATGCGATCGCTGCGGCCGATCTTAGCCAGACGAGCGGGTTCGGCCCATTCGGACCGCA | |
| | AGGAATCGGTCAATACACTACATGGCGTGATTTCATATGCGCGATTGCTGATCCCCATGT | |
| | GTATCACTGGCAAACTGTGATGGACGACACCGTCAGTGCGTCCGTCGCGCAGGCTCTCGA | |
| | TGAGCTGATGCTTTGGGCCGAGGACTGCCCCGAAGTCCGGCACCTCGTGCACGCGGATTT | |
| | CGGCTCCAACAATGTCCTGACGGACAATGGCCGCATAACAGCGGTCATTGACTGGAGCGA | |
| | GGCGATGTTCGGGGATTCCCAATACGAGGTCGCCAACATCTTCTTCTGGAGGCCGTGGTT | |
| | GGCTTGTATGGAGCAGCAGACGCGCTACTTCGAGCGGAGGCATCCGGAGCTTGCAGGATC | |
| | GCCGCGGCTCCGGGCGTATATGCTCCGCATTGGTCTTGACCAACTCTATCAGAGCTTGGT | |
| | TGACGGCAATTTCGATGATGCAGCTTGGGCGCAGGGTCGATGCGACGCAATCGTCCGATC | |
| | CGGAGCCGGGACTGTCGGGCGTACACAAATCGCCCGCAGAAGCGCGGCCGTCTGGACCGA | |
| | TGGCTGTGTAGAAGTACTCGCCGATAGTGGAAACCGACGCCCCAGCACTCGTCCGAGGGC | |
| | AAAGGAATAGGCGGGACTCTGGGGTTCGAAATGACCGACCAAGCGACGCCCAACCTGCCA | |
| | TCACGAGATTTCGATTCCACCGCCGCCTTCTATGAAAGGTTGGGCTTCGGAATCGTTTTC | |
| | CGGGACGCCGGCTGGATGATCCTCCAGCGCGGGGATCTCATGCTGGAGTTCTTCGCCCAC | |
| | GGGATCTCTGCGGAACAGGCGGTCGAAGGTGCCGATATCATTACGACAGCAACGGCCGAC | |
| | AAGCACAACGCCACGATCCTGAGCGACAATATGATCGGGCCCCGATCGTTCAAACATTTG | |
| | GCAATAAAGTTTCTTAAGATTGAATCCTGTTGCCGGTCTTGCGATGATTATCATATAATT | |
| | TCTGTTGAATTACGTTAAGCATGTAATAATTAACATGTAATGCATGACGTTATTTATGAG | |
| | ATGGGTTTTTATGATTAGAGTCCCGCAATTATACATTTAATACGCGATAGAAAACAAAAT | |
| | ATAGCGCGCAAACTAGGATAAATTATCGCGCGCGGTGTCATCTATGTTACTAGATCGGGA | |
| | ATTTGGGCCATCGCCCTGATAGACGGTTTTTCGCCCTTTGACGTTGGAGTCCACGTTCTT | |
| | TAATAGTGGACTCTTGTTCCAAACTGGAACAACACTCAACCCTATCTCGGGCTATTCTTT | |
| | TGATTTATAAGGGATTTTGCCGATTTCGGAACCACCATCAAACAGGATTTTCGCCTGCTG | |
| | GGGCAAACCAGCGTGGACCGCTTGCTGCAACTCTCTCAGGGCCAGGCGGTGAAGGGCAAT | |
| | CAGCTGTTGCCCGTCTCACTGGTGAAAAGAAAAACCACCCCAGTACATTAAAAACGTCCG | |
| | CAATGTGTTATTAAGTTGTCTAAGCGTCAATTTGTTTACACCACAATATATCCTGCCACA | |
| | AGCTAGCTTTCTGAGCCGCCGATTTTCCTCCTCGAGTTGGATGAACTCGCCGAGTTCATC | |
| | GTCAACTGAAACAGACACGGCCGGATTCTGTGAGACAGGTTGAACCGCAGCTCTCTTCCA | |
| | TTGATAATAGGTCTGAACGGAAATACCCACGATCTTAACGGCGTCCTTCAAGGTTGCGCC | |
| | GCCAGCGACCTGAGCTTCGATTTGACCGATCTTCTCCAGTTTTTCTCGGTTGCTGAGGCC | |
| | GCGGGTTTTCGGCTTCACGGATTTGAACGATCCCGTGCGGGCTGTTTCGGCTGGTGCTTT | |
| | CTTTGCTCTTCTACCTCTAGGAGCAGCCGGCTCAACTTCGGCAGCAGCAGTACCGTCCGG | |
| | CGGATTCTGGATCTCTTCGTCAGCCATTAATCGTCCTCTGTGTGGGTTATTGCTTTGTCT | |
| | GCCAGCTCGATCCAAGAGTCAACGTTTGTGCCTAGGGCAGTAAATAGGCAGTGCTCCGCG | |
| | ACTACATGCCTCGGCCGGCAAAATACCGCCGCATGTAGAGCAGGCTCTCCTTCACGATCA | |
| | ACGATCGGCATGGGGCCTTCGTGCTTGTTGAGTAATGTTATCGCTCCCATCAGAGCACGC | |
| | TTGGTACTCCGGGAATCGGATGGTCTGTCGATCATCCAAAAAACGCTCATGTTTTCAACC | |
| | TATTAGGTCTGTGGTCAGCTGACCACAGACCATCCTGCTCCATACTCGCTAATTCTAGCC | |
| | AAACCGCAACGTCCCCTGCCCGCTAGCCTTCAAGAGCGCCATTATCATCGGGCCAAGTGA | |
| | AAACTTCCCGAGTTCGCTCCGCCGTGTCAGATCTCGGAGATAGCCCCCAGGCGAATTGAT | |
| | GAAGTTCGCTCGCTCCAAAATGCACGCCATCGCTGCTGCCGCATTCTCCGGTCCCATTGC | |
| | CTCACACGCGTCTTGGTAAGCCGACGGGCTGACCCCCAGCATAGACCGAACCACCACCGC | |
| | AGCCGACATGAGGTCACGCCAGCTAGCAACCGCACCGCTCGGCCCATAATTGCCAATGGT | |
| | CGGGCACGCTTTCAGGATCATCCCGAGGGGGAACGCTTTTATCGGCTCGCTCCTTGCCCG | |
| | GTCTATTTCACTCGGCTTAGCGCCCTGCTCCTTTTCAGAGCGAGGTTCAAGTTCATTAAC | |
| | GGATTCGGGTTTTGAGTTCTGTATGTGCTGCTCGCTCTGGGCAGCATTGGTGCTATTATT | |
| | TTCTGAATTGTCTCTAATTTCCAACCGGTTGATTATCTCTTCCTGGAGCATCCACATCTC | |
| | TTCGAGAATTGACTCTACATCAGCAAGCGTCGGGGCGCGTGGGATTCTACCCACAAGTTC | |
| | CACATAGACTTCCTCGACAGCTTGCCAGTCGCCCTCCGCTCCCTCTTCCATAGCTGCCGT | |
| | AATTAGCTTCCGAACGTCCCGTCGGCAAATCGTCAGACTTTCTTTGGCCATCCTGAATGC | |
| | TGCTCGATCGGCCATCACCTGCTGTGCCATCATCGCTAGCTCTTCGGACCGCGCGAGAAG | |
| | CGGAGACAAATCGAAGCCAAACGCGCGCTCGATCTGACCAGCGCCATCCTTACGAGCGTA | |
| | ACGCTTTCCGTTGGCGCTATCCTTCCGGACGATCAAGCCTGACTCCACGAGCATGGCGAT | |
| | GTGCCTACGCAAAGTCGCGCCAGCCATCCCATGCGCCCGAAGGGCAAGCTGAGCATTCGA | |
| | CGGGAAGACGATCAGCTGTGCCTCCTGACGCAACTCCGTTTCCGGGTGAAAGCTCAATAG | |
| | CGCATCAAGGACGGCAAGACTGTTGGACTGGATTCCAAGTAGTTCCATGGCCGCGGACGC | |
| | GTCCCTAAAGACCTTCCACTTGTCCGCTGTCTTGCCTTGTTTGATATCGGCCAGCGCCGT | |
| | CTGGCGCCGCACAAGCGCAAGCGTCATTGGCCGCCGCCCGAATGGCGTCGTTACACTTCC | |
| | TGTCTGCATCATCTTTCACCTTTCAGCAGGCAAAGGAAATCAGCTCACCAAAACGGCGCT | |
| | AAAAACTCTTGACGAGGATTCGAGGAAATGCGATTCTGTTCGCGCTAGAGAGACAGAAGG | |
| | GCTTCCGCGACGGCGACGTTGAGGGGGCTCTTTTCTTTTGCGGTTTACTCTCCCCGTTTC | |
| | CGTTGGTTCTCAGCGTGGTACGCTTGATACAGCGCTGGCACATGATCGAGCACGAAGGTC | |
| | GCAAAATCGGGCGTCGCCTTCCTGTCAATCGTGATTTCCAGTTTGGCCTTGCTCTGCGTC | |
| | ACCTGTGCAATTCTGGTGCCGTCTGGGGTGGCCATGACCTCGGGAAGTCCACGCGCAACC | |
| | CGACTGGGCTTCAGACTAGCGATCACCGCCTTGAATCGTTCTGCCGATGGCAGCGCTTGA | |
| | ACTTCCTCCGACATAGCATATTTAGCCACGTCGGCCGGTGAAGAAACTTTCTCAATCAGC | |
| | TCGGCAAGTTGTTGCCAACTCGGCCGTCCAACACCAGGAGCGGCACCAATAGCATCGGTC | |
| | AGTTCAGAGGGGAGGGCGTCAACGAGCAGAAGCATCTTGGACAAATTGCTCTTGTCGATC | |
| | GACATCGCGGCGATGACAATCTCTCGAGAAAACTGCCTGTTCAGGCGATGTGCGAAGCGC | |
| | GCCTTTTCGATGAAGGTAAGATCTTCGCGCACATTGTTTTCCTGACCCTGTGCTACGACC | |
| | ACTTGCTCGTCCGTCAGTTCGCGAACGACCGCCCTGACCGGAAGTCCGAGTTCTGAAACG | |
| | GCGCGTAGCCGGCGGTGGCCGAAGGCAACCTGATATCGGCCCGGCTGGCTCGGATGCGGT | |
| | CGCACAAGGATTGGGACTTGCTGTCCTTGTTCCCGGATCGAAGTAAGGAGCCCGTCAATG | |
| | TCCCCTCGCATACGATCCTGCACGAAAGACGGTTCTATTGACGAGGCATCCAACTCTATC | |
| | ACTGCCTGACCTTCAGCGAGACGCCGCTCGATCTCTTCGGCACGGCTAAGACGATCGTTT | |
| | TGCTCTCGCAGTGCGTTACCAATGTTCGCTGTGAGCTTCGTTGCCGGATCGCGCTCCTTC | |
| | CTTGTTACGCCGAGGAGCGGCATGGAGCGGTTCTTTGCCGTCCTATTGTCGGCGGGCGAC | |
| | GTCTCAGGGGCGTCAGTTGAGACGCCAAGGATGTGCTTCCGGCTCATGTGGGCCTACCCC | |
| | ATGCTTTTTTGATCAGTGTTTCGATCTCGTCGTTGACGGCGTTCATCGCCTCCAAGGCTC | |
| | GATCATAGGTCGAGCGCGTGAACAGGCCACGCTCCACTTCGAATAGAGTCTGGTTTGTCA | |
| | GGCCAGCGTCCGAAACCGCGGTGGTTTTAAGCATCGGAAAATTGAGGACATTTTCGCCAA | |
| | AAATCGACCGCAGATAACCTACCATTTGGTTCTGTGGTCCGTCGCTCGGTTCGAAACGGG | |
| | TTATCAGATAGCGCATCCAATTAAACTTGAACTTGGCGCCAGCATTCTCGATTTCACGCA | |
| | AAAGGTTCGATGTCATTGCCAGAAACTGGTTCATCGACATCACATCCAGCATCTGCGGAT | |
| | GGACCGTGACAAGAATGGACGTCGCCGCAGTCAATGCGGATAGCGTGAGATACCCAAGCT | |
| | GGGGAGGGCAGTCGATGACCACGACGTCATAGTTATCCGCGATATCTTCAATTACTTGGC | |
| | TGATGCGACCATAAAAGAGCGTGTCGCCCTCTTTGCGGTTCATCAGCGCGCGTGGCGTAT | |
| | CGTGTTCAAACTCCATCAGCTCAAGGTTACCAGGAATCAGGTGGAGGTCGGGAATGTAAG | |
| | TCCCTCGGACGACTCGTTCGATTGCCACCTGCTCATCATCATACCTTATAGCGCCGTAGA | |
| | GCGTTTCGTTCGGGCCAACGTCCGTCTCCGGTTGGCTCCCAAAGAGTGCAGAAAGGCTCG | |
| | CTTGAGGATCGAGATCAATGGCCAAGACTCGATATCCGCGCATAGCGAGGTACTGCGCCA | |
| | GATGCGCGGCGGTGGTGGTCTTACCCGACCCACCTTTGAAATTCATCACAGAGATAACCT | |
| | GAAGCTGCTCGCCGCCTCGACGATGTGGCAGGTAGCGCCGGTTCCCGCGGCCGACCTGAT | |
| | CCATATACTTCCGAATCACATGGATATCTTCAATTGAGAACATTCGCCTGCCACCTGGGC | |
| | TCATGCTAACATTCAACTCTGGCATCTCAGACGCGGTCTGCCGTAAATATGACTCGCCAA | |
| | CGCCGAGCAGCTTGGACGCCTCCGATGGCCCGAATGTTCGAATACCCTTCTCGGAATGCG | |
| | GCGGGAAAACCTTAAGATGATGTGCTTGAAGTTGGCTCGAGAGGGCATCGGCATGACGCT | |
| | CCATCAAGGCCGTCAACCCTACAACTACAGGCGCTGCTTTTAGGACAGACTTCGCCATCT | |
| | CAAACCCATTCCTTGCCAGTGGCGATATTTTTCGCGAAACTGGAAAAGTTCCGCCGCTGG | |
| | CAATTAGCGCCGATTCTGCTGTTTGGGCAAGAGCTTTTAGGTTAACAGAAGGTTAACGCC | |
| | CTCAGGTCGAAAAACTCCACCCAACTGTTATTTGTATTTATTTCCAATGCCTTAGAGAGA | |
| | TTGCCATTTGAATATGTTCATGTATTGTTTTAGTGATAATCCTACAATCGTAACCCAAAA | |
| | AGAGGTCGCCCTCTGCGCGCCGTCGTCCAATATAGGCGAAGTCACCCTTGCGACTCAGGC | |
| | GGATTCTACCTTGTAGGATCGAGTCAGGCAACTATGGATGAACGAAATAGACAGATCGCT | |
| | GAGATAGGTGCCTCACTGATTAAGCATTGGTAACTGTCAGACCAAGTTTACTCATATATA | |
| | CTTTAGATTGATTTAAAACTTCATTTTTAATTTAAAAGGATCTAGGTGAAGATCCTTTTT | |
| | GATAATCTCATGACCAAAATCCCTTAACGTGAGTTTTCGTTCCACTGAGCGTCAGACCCC | |
| | GTAGAAAAGATCAAAGGATCTTCTTGAGATCCTTTTTTTCTGCGCGTAATCTGCTGCTTG | |
| | CAAACAAAAAAACCACCGCTACCAGCGGTGGTTTGTTTGCCGGATCAAGAGCTACCAACT | |
| | CTTTTTCCGAAGGTAACTGGCTTCAGCAGAGCGCAGATACCAAATACTGTTCTTCTAGTG | |
| | TAGCCGTAGTTAGGCCACCACTTCAAGAACTCTGTAGCACCGCCTACATACCTCGCTCTG | |
| | CTAATCCTGTTACCAGTGGCTGCTGCCAGTGGCGATAAGTCGTGTCTTACCGGGTTGGAC | |
| | TCAAGACGATAGTTACCGGATAAGGCGCAGCGGTCGGGCTGAACGGGGGGTTCGTGCACA | |
| | CAGCCCAGCTTGGAGCGAACGACCTACACCGAACTGAGATACCTACAGCGTGAGCTATGA | |
| | GAAAGCGCCACGCTTCCCGAAGGGAGAAAGGCGGACAGGTATCCGGTAAGCGGCAGGGTC | |
| | GGAACAGGAGAGCGCACGAGGGAGCTTCCAGGGGGAAACGCCTGGTATCTTTATAGTCCT | |
| | GTCGGGTTTCGCCACCTCTGACTTGAGCGTCGATTTTTGTGATGCTCGTCAGGGGGGCGG | |
| | AGCCTATGGAAAAACGCCAGCAACGCGGCCTTTTTACGGTTCCTGGCCTTTTGCTGGCCT | |
| | TTTGCTCA | |
| SPCas9- | atggataagaagtactctatcggactcgatatcggaactaactctgtgggatgggctgtg | 46 |
| NLS | atcaccgatgagtacaaggtgccatctaagaagttcaaggttctcggaaacaccgatagg | |
| | cactctatcaagaaaaaccttatcggtgctctcctcttcgattctggtgaaactgctgag | |
| | gctaccagactcaagagaaccgctagaagaaggtacaccagaagaaagaacaggatctgc | |
| | tacctccaagagatcttctctaacgagatggctaaagtggatgattcattcttccacagg | |
| | ctcgaagagtcattcctcgtggaagaagataagaagcacgagaggcaccctatcttcgga | |
| | aacatcgttgatgaggtggcataccacgagaagtaccctactatctaccacctcagaaag | |
| | aagctcgttgattctactgataaggctgatctcaggctcatctacctcgctctcgctcac | |
| | atgatcaagttcagaggacacttcctcatcgagggtgatctcaaccctgataactctgat | |
| | gtggataagttgttcatccagctcgtgcagacctacaaccagcttttcgaagagaaccct | |
| | atcaacgcttcaggtgtggatgctaaggctatcctctctgctaggctctctaagtcaaga | |
| | aggcttgagaacctcattgctcagctccctggtgagaagaagaacggacttttcggaaac | |
| | ttgatcgctctctctctcggactcacccctaacttcaagtctaacttcgatctcgctgag | |
| | gatgcaaagctccagctctcaaaggatacctacgatgatgatctcgataacctcctcgct | |
| | cagatcggagatcagtacgctgatttgttcctcgctgctaagaacctctctgatgctatc | |
| | ctcctcagtgatatcctcagagtgaacaccgagatcaccaaggctccactctcagcttct | |
| | atgatcaagagatacgatgagcaccaccaggatctcacacttctcaaggctcttgttaga | |
| | cagcagctcccagagaagtacaaagagattttcttcgatcagtctaagaacggatacgct | |
| | ggttacatcgatggtggtgcatctcaagaagagttctacaagttcatcaagcctatcctc | |
| | gagaagatygatggaaccgaggaactcctcgtgaagctcaatagagaggatcttctcaga | |
| | aagcagaggaccttcgataacggatctatccctcatcagatccacctcggagagttgcac | |
| | gctatccttagaaggcaagaggatttctacccattcctcaaggataacagggaaaagatt | |
| | gagaagattctcaccttcagaatcccttactacgtgggacctctcgctagaggaaactca | |
| | agattcgcttggatgaccagaaagtctgaggaaaccatcaccccttggaacttcgaagag | |
| | gtggtggataagggtgctagtgctcagtctttcatcgagaggatgaccaacttcgataag | |
| | aaccttccaaacgagaaggtgctccctaagcactctttgctctacgagtacttcaccgtg | |
| | tacaacgagttgaccaaggttaagtacgtgaccgagggaatgaggaagcctgcttttttg | |
| | tcaggtgagcaaaagaaggctatcgttgatctcttgttcaagaccaacagaaaggtgacc | |
| | gtgaagcagctcaaagaggattacttcaagaaaatcgagtgcttcgattcagttgagatt | |
| | tctggtgttgaggataggttcaacgcatctctcggaacctaccacgatctcctcaagatc | |
| | attaaggataaggatttcttggataacgaggaaaacgaggatatcttggaggatatcgtt | |
| | cttaccctcaccctctttgaagatagagagatgattgaagaaaggctcaagacctacgct | |
| | catctcttcgatgataaggtgatgaagcagttgaagagaagaagatacactggttgggga | |
| | aggctctcaagaaagctcattaacggaatcagggataagcagtctggaaagacaatcctt | |
| | gatttcctcaagtctgatggattcgctaacagaaacttcatgcagctcatccacgatgat | |
| | tctctcacctttaaagaggatatccagaaggctcaggtttcaggacagggtgatagtctc | |
| | catgagcatatcgctaacctcgctggatctcctgcaatcaagaagggaatcctccagact | |
| | gtgaaggttgtggatgagttggtgaaggtgatgggaaggcataagcctgagaacatcgtg | |
| | atcgaaatggctagagagaaccagaccactcagaagggacagaagaactctagggaaagg | |
| | atgaagaggatcgaggaaggtatcaaagagcttggatctcagatcctcaaagagcaccct | |
| | gttgagaacactcagctccagaatgagaagctctacctctactacctccagaacggaagg | |
| | gatatgtatgtggatcaagagttggatatcaacaggctctctgattacgatgttgatcat | |
| | atcgtgccacagtcattcttgaaggatgattctatcgataacaaggtgctcaccaggtct | |
| | gataagaacaggggtaagagtgataacgtgccaagtgaagaggttgtgaagaaaatgaag | |
| | aactattggaggcagctcctcaacgctaagctcatcactcagagaaagttcgataacttg | |
| | actaaggctgagaggggaggactctctgaattggataaggcaggattcatcaagaggcag | |
| | cttgtggaaaccaggcagatcactaagcacgttgcacagatcctcgattctaggatgaac | |
| | accaagtacgatgagaacgataagttgatcagggaagtgaaggttatcaccctcaagtca | |
| | aagctcgtgtctgatttcagaaaggatttccaattctacaaggtgagggaaatcaacaac | |
| | taccaccacgctcacgatgcttaccttaacgctgttgttggaaccgctctcatcaagaag | |
| | tatcctaagctcgagtcagagttcgtgtacggtgattacaaggtgtacgatgtgaggaag | |
| | atgatcgctaagtctgagcaagagatcggaaaggctaccgctaagtatttcttctactct | |
| | aacatcatgaatttcttcaagaccgagattaccctcgctaacggtgagatcagaaagagg | |
| | ccactcatcgagacaaacggtgaaacaggtgagatcgtgtgggataagggaagggatttc | |
| | gctaccgttagaaaggtgctctctatgccacaggtgaacatcgttaagaaaaccgaggtg | |
| | cagaccggtggattctctaaagagtctatcctccctaagaggaactctgataagctcatt | |
| | gctaggaagaaggattgggaccctaagaaatacggtggtttcgattctcctaccgtggct | |
| | tactctgttctcgttgtggctaaggttgagaagggaaagagtaagaagctcaagtctgtt | |
| | aaggaacttctcggaatcactatcatggaaaggtcatctttcgagaagaacccaatcgat | |
| | ttcctcgaggctaagggatacaaagaggttaagaaggatctcatcatcaagctcccaaag | |
| | tactcactcttcgaactcgagaacggtagaaagaggatgctcgcttctgctggtgagctt | |
| | caaaagggaaacgagcttgctctcccatctaagtacgttaactttctttacctcgcttct | |
| | cactacgagaagttgaagggatctccagaagataacgagcagaagcaacttttcgttgag | |
| | cagcacaagcactacttggatgagatcatcgagcagatctctgagttctctaaaagggtg | |
| | atcctcgctgatgcaaacctcgataaggtgttgtctgcttacaacaagcacagagataag | |
| | cctatcagggaacaggcagagaacatcatccatctcttcacccttaccaacctcggtgct | |
| | cctgctgctttcaagtacttcgatacaaccatcgataggaagagatacacctctaccaaa | |
| | gaagtgctcgatgctaccctcatccatcagtctatcactggactctacgagactaggatc | |
| | gatctctcacagctcggtggtgattcaagggctgatcctaagaagaagaggaaggtttga | |
| Cas9 | CTTTTTTTCTTCTTCTTCGTTCATACAGTTTTTTTTTGTTTATCAGCTTACATTTTCTTG | 47 |
| guideblock | AACCGTAGCTTTCGTTTTCTTCTTTTTAACTTTCCATTCGGAGTTTTTGTATCTTGTTTC | |
| | ATAGTTTGTCCCAGGATTAGAATGATTAGGCATCGAACCTTCAAGAATTTGATTGAATAA | |
| | AACATCTTCATTCTTAAGATATGAAGATAATCTTCAAAAGGCCCCTGGGAATCTGAAAGA | |
| | AGAGAAGCAGGCCCATTTATATGGGAAAGAACAATAGTATTTCTTATATAGGCCCATTTA | |
| | AGTTGAAAACAATCTTCAAAAGTCCCACATCGCTTAGATAAGAAAACGAAGCTGAGTTTA | |
| | TATACAGCTAGAGTCGAAGTAGTGATTGnnnnnnnnnnnnnnnnnnnnGTTTTAGAGCTA | |
| | GAAATAGCAAGTTAAAATAAGGCTAGTCCGTTATCAACTTGAAAAAGTGGCACCGAGTCG | |
| | GTGCTTTTTTTCTAGACCCAGCTTTCTTGTACAAAGTTGGCATTA | |
| pUC-Cas9- | AAGCTTGCATGCCTGCAGGTCCCCAGATTAGCCTTTTCAATTTCAGAAAGAATGCTAACC | 48 |
| HygroR | CACAGATGGTTAGAGAGGCTTACGCAGCAGGTCTCATCAAGACGATCTACCCGAGCAATA | |
| | ATCTCCAGGAAATCAAATACCTTCCCAAGAAGGTTAAAGATGCAGTCAAAAGATTCAGGA | |
| | CTAACTGCATCAAGAACACAGAGAAAGATATATTTCTCAAGATCAGAAGTACTATTCCAG | |
| | TATGGACGATTCAAGGCTTGCTTCACAAACCAAGGCAAGTAATAGAGATTGGAGTCTCTA | |
| | AAAAGGTAGTTCCCACTGAATCAAAGGCCATGGAGTCAAAGATTCAAATAGAGGACCTAA | |
| | CAGAACTCGCCGTAAAGACTGGCGAACAGTTCATACAGAGTCTCTTACGACTCAATGACA | |
| | AGAAGAAAATCTTCGTCAACATGGTGGAGCACGACACACTTGTCTACTCCAAAAATATCA | |
| | AAGATACAGTCTCAGAAGACCAAAGGGCAATTGAGACTTTTCAACAAAGGGTAATATCCG | |
| | GAAACCTCCTCGGATTCCATTGCCCAGCTATCTGTCACTTTATTGTGAAGATAGTGGAAA | |
| | AGGAAGGTGGCTCCTACAAATGCCATCATTGCGATAAAGGAAAGGCCATCGTTGAAGATG | |
| | CCTCTGCCGACAGTGGTCCCAAAGATGGACCCCCACCCACGAGGAGCATCGTGGAAAAAG | |
| | AAGACGTTCCAACCACGTCTTCAAAGCAAGTGGATTGATGTGATATCTCCACTGACGTAA | |
| | GGGATGACGCACAATCCCACTATCCTTCGCAAGACCCTTCCTCTATATAAGGAAGTTCAT | |
| | TTCATTTGGAGAGAACACGGGGGACTCTAGATACATCACAATCACACAAAACTAACAAAA | |
| | GATCAAAAGCAAGTTCTTCACTGTTGATACATatggataagaagtactctatcggactcg | |
| | atatcggaactaactctgtgggatgggctgtgatcaccgatgagtacaaggtgccatcta | |
| | agaagttcaaggttctcggaaacaccgataggcactctatcaagaaaaaccttatcggtg | |
| | ctctcctcttcgattctggtgaaactgctgaggctaccagactcaagagaaccgctagaa | |
| | gaaggtacaccagaagaaagaacaggatctgctacctccaagagatcttctctaacgaga | |
| | tggctaaagtggatgattcattcttccacaggctcgaagagtcattcctcgtggaagaag | |
| | ataagaagcacgagaggcaccctatcttcggaaacatcgttgatgaggtggcataccacg | |
| | agaagtaccctactatctaccacctcagaaagaagctcgttgattctactgataaggctg | |
| | atctcaggctcatctacctcgctctcgctcacatgatcaagttcagaggacacttcctca | |
| | tcgagggtgatctcaaccctgataactctgatgtggataagttgttcatccagctcgtgc | |
| | agacctacaaccagcttttcgaagagaaccctatcaacgcttcaggtgtggatgctaagg | |
| | ctatcctctctgctaggctctctaagtcaagaaggcttgagaacctcattgctcagctcc | |
| | ctggtgagaagaagaacggacttttcggaaacttgatcgctctctctctcggactcaccc | |
| | ctaacttcaagtctaacttcgatctcgctgaggatgcaaagctccagctctcaaaggata | |
| | cctacgatgatgatctcgataacctcctcgctcagatcggagatcagtacgctgatttgt | |
| | tcctcgctgctaagaacctctctgatgctatcctcctcagtgatatcctcagagtgaaca | |
| | ccgagatcaccaaggctccactctcagcttctatgatcaagagatacgatgagcaccacc | |
| | aggatctcacacttctcaaggctcttgttagacagcagctcccagagaagtacaaagaga | |
| | ttttcttcgatcagtctaagaacggatacgctggttacatcgatggtggtgcatctcaag | |
| | aagagttctacaagttcatcaagcctatcctcgagaagatggatggaaccgaggaactcc | |
| | tcgtgaagctcaatagagaggatcttctcagaaagcagaggaccttcgataacggatcta | |
| | tccctcatcagatccacctcggagagttgcacgctatccttagaaggcaagaggatttct | |
| | acccattcctcaaggataacagggaaaagattgagaagattctcaccttcagaatccctt | |
| | actacgtgggacctctcgctagaggaaactcaagattcgcttggatgaccagaaagtctg | |
| | aggaaaccatcaccccttggaacttcgaagaggtggtggataagggtgctagtgctcagt | |
| | ctttcatcgagaggatgaccaacttcgataagaaccttccaaacgagaaggtgctcccta | |
| | agcactctttgctctacgagtacttcaccgtgtacaacgagttgaccaaggttaagtacg | |
| | tgaccgagggaatgaggaagcctgcttttttgtcaggtgagcaaaagaaggctatcgttg | |
| | atctcttgttcaagaccaacagaaaggtgaccgtgaagcagctcaaagaggattacttca | |
| | agaaaatcgagtgcttcgattcagttgagatttctggtgttgaggataggttcaacgcat | |
| | ctctcggaacctaccacgatctcctcaagatcattaaggataaggatttcttggataacg | |
| | aggaaaacgaggatatcttggaggatatcgttcttaccctcaccctctttgaagatagag | |
| | agatgattgaagaaaggctcaagacctacgctcatctcttcgatgataaggtgatgaagc | |
| | agttgaagagaagaagatacactggttggggaaggctctcaagaaagctcattaacggaa | |
| | tcagggataagcagtctggaaagacaatccttgatttcctcaagtctgatggattcgcta | |
| | acagaaacttcatgcagctcatccacgatgattctctcacctttaaagaggatatccaga | |
| | aggctcaggtttcaggacagggtgatagtctccatgagcatatcgctaacctcgctggat | |
| | ctcctgcaatcaagaagggaatcctccagactgtgaaggttgtggatgagttggtgaagg | |
| | tgatgggaaggcataagcctgagaacatcgtgatcgaaatggctagagagaaccagacca | |
| | ctcagaagggacagaagaactctagggaaaggatgaagaggatcgaggaaggtatcaaag | |
| | agcttggatctcagatcctcaaagagcaccctgttgagaacactcagctccagaatgaga | |
| | agctctacctctactacctccagaacggaagggatatgtatgtggatcaagagttggata | |
| | tcaacaggctctctgattacgatgttgatcatatcgtgccacagtcattcttgaaggatg | |
| | attctatcgataacaaggtgctcaccaggtctgataagaacaggggtaagagtgataacg | |
| | tgccaagtgaagaggttgtgaagaaaatgaagaactattggaggcagctcctcaacgcta | |
| | agctcatcactcagagaaagttcgataacttgactaaggctgagaggggaggactctctg | |
| | aattggataaggcaggattcatcaagaggcagcttgtggaaaccaggcagatcactaagc | |
| | acgttgcacagatcctcgattctaggatgaacaccaagtacgatgagaacgataagttga | |
| | tcagggaagtgaaggttatcaccctcaagtcaaagctcgtgtctgatttcagaaaggatt | |
| | tccaattctacaaggtgagggaaatcaacaactaccaccacgctcacgatgcttacctta | |
| | acgctgttgttggaaccgctctcatcaagaagtatcctaagctcgagtcagagttcgtgt | |
| | acggtgattacaaggtgtacgatgtgaggaagatgatcgctaagtctgagcaagagatcg | |
| | gaaaggctaccgctaagtatttcttctactctaacatcatgaatttcttcaagaccgaga | |
| | ttaccctcgctaacggtgagatcagaaagaggccactcatcgagacaaacggtgaaacag | |
| | gtgagatcgtgtgggataagggaagggatttcgctaccgttagaaaggtgctctctatgc | |
| | cacaggtgaacatcgttaagaaaaccgaggtgcagaccggtggattctctaaagagtcta | |
| | tcctccctaagaggaactctgataagctcattgctaggaagaaggattgggaccctaaga | |
| | aatacggtggtttcgattctcctaccgtggcttactctgttctcgttgtggctaaggttg | |
| | agaagggaaagagtaagaagctcaagtctgttaaggaacttctcggaatcactatcatgg | |
| | aaaggtcatctttcgagaagaacccaatcgatttcctcgaggctaagggatacaaagagg | |
| | ttaagaaggatctcatcatcaagctcccaaagtactcactcttcgaactcgagaacggta | |
| | gaaagaggatgctcgcttctgctggtgagcttcaaaagggaaacgagcttgctctcccat | |
| | ctaagtacgttaactttctttacctcgcttctcactacgagaagttgaagggatctccag | |
| | aagataacgagcagaagcaacttttcgttgagcagcacaagcactacttggatgagatca | |
| | tcgagcagatctctgagttctctaaaagggtgatcctcgctgatgcaaacctcgataagg | |
| | tgttgtctgcttacaacaagcacagagataagcctatcagggaacaggcagagaacatca | |
| | tccatctcttcacccttaccaacctcggtgctcctgctgctttcaagtacttcgatacaa | |
| | ccatcgataggaagagatacacctctaccaaagaagtgctcgatgctaccctcatccatc | |
| | agtctatcactggactctacgagactaggatcgatctctcacagctcggtggtgattcaa | |
| | gggctgatcctaagaagaagaggaaggtttgaGTCGACAATTCTGAATCAACAACTCTCC | |
| | TGGCGCACCATCGTCGGCTACAGCCTCGGGAATTGCTACCGAGCTCTTATGAAGATGAAG | |
| | ATGAAATATTTGGTGTGTCAAATAAAAAGCTAGCTTGTGTGCTTAAGTTTGTGTTTTTTT | |
| | CTTGGCTTGTTGTGTTATGAATTTGTGGCTTTTTCTAATATTAAATGAATGTAAGATCTC | |
| | ATTATAATGAATAAACAAATGTTTCTATAATCCATTGTGAATGTTTTGTTGGATCTCTTC | |
| | GCATATAACTACTGTATGTGCTATGGTATGGACTATGGAATATGATTAAAGATAAGGAAT | |
| | TGGTACCTGCGGCTTTTTTTCTTCTTCTTCGTTCATACAGTTTTTTTTTGTTTATCAGCT | |
| | TACATTTTCTTGAACCGTAGCTTTCGTTTTCTTCTTTTTAACTTTCCATTCGGAGTTTTT | |
| | GTATCTTGTTTCATAGTTTGTCCCAGGATTAGAATGATTAGGCATCGAACCTTCAAGAAT | |
| | TTGATTGAATAAAACATCTTCATTCTTAAGATATGAAGATAATCTTCAAAAGGCCCCTGG | |
| | GAATCTGAAAGAAGAGAAGCAGGCCCATTTATATGGGAAAGAACAATAGTATTTCTTATA | |
| | TAGGCCCATTTAAGTTGAAAACAATCTTCAAAAGTCCCACATCGCTTAGATAAGAAAACG | |
| | AAGCTGAGTTTATATACAGCTAGAGTCGAAGTAGTGATTGnnnnnnnnnnnnnnnnnnnn | |
| | GTTTTAGAGCTAGAAATAGCAAGTTAAAATAAGGCTAGTCCGTTATCAACTTGAAAAAGT | |
| | GGCACCGAGTCGGTGCTTTTTTTCTAGACCCAGCTTTCTTGTACAAAGTTGGCATTACGC | |
| | TGGATCCCGGGAATTCGTAATCATGGTCATAGCTGTTTCCTGTGTGAAATTGTTATCCGC | |
| | TCACAATTCCACACAACATACGAGCCGGAAGCATAAAGTGTAAAGCCTGGGGTGCCTAAT | |
| | GAGTGAGCTAACTCACATTAATTGCGTTGCGCTCACTGCCCGCTTTCCAGTCGGGAAACC | |
| | TGTCGTGCCAGCTGCATTAATGAATCGGCCAACGCGCGGGGAGAGGCGGTTTGCGTATTG | |
| | GCTAGAGCAGCTTGCCAACATGGTGGAGCACGACACTCTCGTCTACTCCAAGAATATCAA | |
| | AGATACAGTCTCAGAAGACCAAAGGGCTATTGAGACTTTTCAACAAAGGGTAATATCGGG | |
| | AAACCTCCTCGGATTCCATTGCCCAGCTATCTGTCACTTCATCAAAAGGACAGTAGAAAA | |
| | GGAAGGTGGCACCTACAAATGCCATCATTGCGATAAAGGAAAGGCTATCGTTCAAGATGC | |
| | CTCTGCCGACAGTGGTCCCAAAGATGGACCCCCACCCACGAGGAGCATCGTGGAAAAAGA | |
| | AGACGTTCCAACCACGTCTTCAAAGCAAGTGGATTGATGTGATATCTCCACTGACGTAAG | |
| | GGATGACGCACAATCCCACTATCCTTCGCAAGACCTTCCTCTATATAAGGAAGTTCATTT | |
| | CATTTGGAGAGGACACGCTGGAATTCTAGTATACTAAACCATGAAAAAGCCTGAACTCAC | |
| | CGCGACGTCTGTCGAGAAGTTTCTGATCGAAAAGTTCGACAGCGTCTCCGACCTGATGCA | |
| | GCTCTCGGAGGGCGAAGAATCTCGTGCTTTCAGCTTCGATGTAGGAGGGCGTGGATATGT | |
| | CCTGCGGGTAAATAGCTGCGCCGATGGTTTCTACAAAGATCGTTATGTTTATCGGCACTT | |
| | TGCATCGGCCGCGCTCCCGATTCCGGAAGTGCTTGACATTGGGGAATTCAGCGAGAGCCT | |
| | GACCTATTGCATCTCCCGCCGTGCACAGGGTGTCACGTTGCAAGACCTGCCTGAAACCGA | |
| | ACTGCCCGCTGTTCTGCAGCCGGTCGCGGAGGCCATGGATGCGATCGCTGCGGCCGATCT | |
| | TAGCCAGACGAGCGGGTTCGGCCCATTCGGACCGCAAGGAATCGGTCAATACACTACATG | |
| | GCGTGATTTCATATGCGCGATTGCTGATCCCCATGTGTATCACTGGCAAACTGTGATGGA | |
| | CGACACCGTCAGTGCGTCCGTCGCGCAGGCTCTCGATGAGCTGATGCTTTGGGCCGAGGA | |
| | CTGCCCCGAAGTCCGGCACCTCGTGCACGCGGATTTCGGCTCCAACAATGTCCTGACGGA | |
| | CAATGGCCGCATAACAGCGGTCATTGACTGGAGCGAGGCGATGTTCGGGGATTCCCAATA | |
| | CGAGGTCGCCAACATCTTCTTCTGGAGGCCGTGGTTGGCTTGTATGGAGCAGCAGACGCG | |
| | CTACTTCGAGCGGAGGCATCCGGAGCTTGCAGGATCGCCGCGGCTCCGGGCGTATATGCT | |
| | CCGCATTGGTCTTGACCAACTCTATCAGAGCTTGGTTGACGGCAATTTCGATGATGCAGC | |
| | TTGGGCGCAGGGTCGATGCGACGCAATCGTCCGATCCGGAGCCGGGACTGTCGGGCGTAC | |
| | ACAAATCGCCCGCAGAAGCGCGGCCGTCTGGACCGATGGCTGTGTAGAAGTACTCGCCGA | |
| | TAGTGGAAACCGACGCCCCAGCACTCGTCCGAGGGCAAAGGAATAGGCGGGACTCTGGGG | |
| | TTCGAAATGACCGACCAAGCGACGCCCAACCTGCCATCACGAGATTTCGATTCCACCGCC | |
| | GCCTTCTATGAAAGGTTGGGCTTCGGAATCGTTTTCCGGGACGCCGGCTGGATGATCCTC | |
| | CAGCGCGGGGATCTCATGCTGGAGTTCTTCGCCCACGGGATCTCTGCGGAACAGGCGGTC | |
| | GAAGGTGCCGATATCATTACGACAGCAACGGCCGACAAGCACAACGCCACGATCCTGAGC | |
| | GACAATATGATCGGGCCCCGATCGTTCAAACATTTGGCAATAAAGTTTCTTAAGATTGAA | |
| | TCCTGTTGCCGGTCTTGCGATGATTATCATATAATTTCTGTTGAATTACGTTAAGCATGT | |
| | AATAATTAACATGTAATGCATGACGTTATTTATGAGATGGGTTTTTATGATTAGAGTCCC | |
| | GCAATTATACATTTAATACGCGATAGAAAACAAAATATAGCGCGCAAACTAGGATAAATT | |
| | ATCGCGCGCGGTGTCATCTATGTTACTAGATCtcttccgcttcctcgctcactgactcgc | |
| | tgcgctcggtcgttcggctgcggcgagcggtatcagctcactcaaaggcggtaatacggt | |
| | tatccacagaatcaggggataacgcaggaaagaacatgtgagcaaaaggccagcaaaagg | |
| | ccaggaaccgtaaaaaggccgcgttgctggcgtttttccataggctccgcccccctgacg | |
| | agcatcacaaaaatcgacgctcaagtcagaggtggcgaaacccgacaggactataaagat | |
| | accaggcgtttccccctggaagctccctcgtgcgctctcctgttccgaccctgccgctta | |
| | ccggatacctgtccgcctttctcccttcgggaagcgtggcgctttctcatagctcacgct | |
| | gtaggtatctcagttcggtgtaggtcgttcgctccaagctgggctgtgtgcacgaacccc | |
| | ccgttcagcccgaccgctgcgccttatccggtaactatcgtcttgagtccaacccggtaa | |
| | gacacgacttatcgccactggcagcagccactggtaacaggattagcagagcgaggtatg | |
| | taggcggtgctacagagttcttgaagtggtggcctaactacggctacactagaagaacag | |
| | tatttggtatctgcgctctgctgaagccagttaccttcggaaaaagagttggtagctctt | |
| | gatccggcaaacaaaccaccgctggtagcggtggtttttttgtttgcaagcagcagatta | |
| | cgcgcagaaaaaaaggatctcaagaagatcctttgatcttttctacggggtctgacgctc | |
| | agtggaacgaaaactcacgttaagggattttggtcatgagattatcaaaaaggatcttca | |
| | cctagatccttttaaattaaaaatgaagttttaaatcaatctaaagtatatatgagtaaa | |
| | cttggtctgacagttaccaatgcttaatcagtgaggcacctatctcagcgatctgtctat | |
| | ttcgttcatccatagttgcctgactccccgtcgtgtagataactacgatacgggagggct | |
| | taccatctggccccagtgctgcaatgataccgcgagacccacgctcaccggctccagatt | |
| | tatcagcaataaaccagccagccggaagggccgagcgcagaagtggtcctgcaactttat | |
| | ccgcctccatccagtctattaattgttgccgggaagctagagtaagtagttcgccagtta | |
| | atagtttgcgcaacgttgttgccattgctacaggcatcgtggtgtcacgctcgtcgtttg | |
| | gtatggcttcattcagctccggttcccaacgatcaaggcgagttacatgatcccccatgt | |
| | tgtgcaaaaaagcggttagctccttcggtcctccgatcgttgtcagaagtaagttggccg | |
| | cagtgttatcactcatggttatggcagcactgcataattctcttactgtcatgccatccg | |
| | taagatgcttttctgtgactggtgagtactcaaccaagtcattctgagaatagtgtatgc | |
| | ggcgaccgagttgctcttgcccggcgtcaatacgggataataccgcgccacatagcagaa | |
| | ctttaaaagtgctcatcattggaaaacgttcttcggggcgaaaactctcaaggatcttac | |
| | cgctgttgagatccagttcgatgtaacccactcgtgcacccaactgatcttcagcatctt | |
| | ttactttcaccagcgtttctgggtgagcaaaaacaggaaggcaaaatgccgcaaaaaagg | |
| | gaataagggcgacacggaaatgttgaatactcatactcttcctttttcaatattattgaa | |
| | gcatttatcagggttattgtctcatgagcggatacatatttgaatgtatttagaaaaata | |
| | aacaaataggggttccgcgcacatttccccgaaaagtgccacctgacgtctaagaaacca | |
| | ttattatcatgacattaacctataaaaataggcgtatcacgaggccctttcgtctcgcgc | |
| | gtttcggtgatgacggtgaaaacctctgacacatgcagctcccggagacggtcacagctt | |
| | gtctgtaagcggatgccgggagcagacaagcccgtcagggcgcgtcagcgggtgttggcg | |
| | ggtgtcggggctggcttaa | |
| pUc- | AAGCTTGCATGCCTGCAGGTCCCCAGATTAGCCTTTTCAATTTCAGAAAGAATGCTAACC | 49 |
| HygroR | CACAGATGGTTAGAGAGGCTTACGCAGCAGGTCTCATCAAGACGATCTACCCGAGCAATA | |
| | ATCTCCAGGAAATCAAATACCTTCCCAAGAAGGTTAAAGATGCAGTCAAAAGATTCAGGA | |
| | CTAACTGCATCAAGAACACAGAGAAAGATATATTTCTCAAGATCAGAAGTACTATTCCAG | |
| | TATGGACGATTCAAGGCTTGCTTCACAAACCAAGGCAAGTAATAGAGATTGGAGTCTCTA | |
| | AAAAGGTAGTTCCCACTGAATCAAAGGCCATGGAGTCAAAGATTCAAATAGAGGACCTAA | |
| | CAGAACTCGCCGTAAAGACTGGCGAACAGTTCATACAGAGTCTCTTACGACTCAATGACA | |
| | AGAAGAAAATCTTCGTCAACATGGTGGAGCACGACACACTTGTCTACTCCAAAAATATCA | |
| | AAGATACAGTCTCAGAAGACCAAAGGGCAATTGAGACTTTTCAACAAAGGGTAATATCCG | |
| | GAAACCTCCTCGGATTCCATTGCCCAGCTATCTGTCACTTTATTGTGAAGATAGTGGAAA | |
| | AGGAAGGTGGCTCCTACAAATGCCATCATTGCGATAAAGGAAAGGCCATCGTTGAAGATG | |
| | CCTCTGCCGACAGTGGTCCCAAAGATGGACCCCCACCCACGAGGAGCATCGTGGAAAAAG | |
| | AAGACGTTCCAACCACGTCTTCAAAGCAAGTGGATTGATGTGATATCTCCACTGACGTAA | |
| | GGGATGACGCACAATCCCACTATCCTTCGCAAGACCCTTCCTCTATATAAGGAAGTTCAT | |
| | TTCATTTGGAGAGAACACGGGGGACTCTAGATACATCACAATCACACAAAACTAACAAAA | |
| | GATCAAAAGCAAGTTCTTCACTGTTGATACATATGCCCGTCGATTAGCAAGTTACTACCG | |
| | GGTCGACAATTCTGAATCAACAACTCTCCTGGCGCACCATCGTCGGCTACAGCCTCGGGA | |
| | ATTGCTACCGAGCTCTTATGAAGATGAAGATGAAATATTTGGTGTGTCAAATAAAAAGCT | |
| | AGCTTGTGTGCTTAAGTTTGTGTTTTTTTCTTGGCTTGTTGTGTTATGAATTTGTGGCTT | |
| | TTTCTAATATTAAATGAATGTAAGATCTCATTATAATGAATAAACAAATGTTTCTATAAT | |
| | CCATTGTGAATGTTTTGTTGGATCTCTTCGCATATAACTACTGTATGTGCTATGGTATGG | |
| | ACTATGGAATATGATTAAAGATAAGGAATTGGTACCTGCGGCCGCTGGATCCCGGGAATT | |
| | CGTAATCATGGTCATAGCTGTTTCCTGTGTGAAATTGTTATCCGCTCACAATTCCACACA | |
| | ACATACGAGCCGGAAGCATAAAGTGTAAAGCCTGGGGTGCCTAATGAGTGAGCTAACTCA | |
| | CATTAATTGCGTTGCGCTCACTGCCCGCTTTCCAGTCGGGAAACCTGTCGTGCCAGCTGC | |
| | ATTAATGAATCGGCCAACGCGCGGGGAGAGGCGGTTTGCGTATTGGCTAGAGCAGCTTGC | |
| | CAACATGGTGGAGCACGACACTCTCGTCTACTCCAAGAATATCAAAGATACAGTCTCAGA | |
| | AGACCAAAGGGCTATTGAGACTTTTCAACAAAGGGTAATATCGGGAAACCTCCTCGGATT | |
| | CCATTGCCCAGCTATCTGTCACTTCATCAAAAGGACAGTAGAAAAGGAAGGTGGCACCTA | |
| | CAAATGCCATCATTGCGATAAAGGAAAGGCTATCGTTCAAGATGCCTCTGCCGACAGTGG | |
| | TCCCAAAGATGGACCCCCACCCACGAGGAGCATCGTGGAAAAAGAAGACGTTCCAACCAC | |
| | GTCTTCAAAGCAAGTGGATTGATGTGATATCTCCACTGACGTAAGGGATGACGCACAATC | |
| | CCACTATCCTTCGCAAGACCTTCCTCTATATAAGGAAGTTCATTTCATTTGGAGAGGACA | |
| | CGCTGGAATTCTAGTATACTAAACCATGAAAAAGCCTGAACTCACCGCGACGTCTGTCGA | |
| | GAAGTTTCTGATCGAAAAGTTCGACAGCGTCTCCGACCTGATGCAGCTCTCGGAGGGCGA | |
| | AGAATCTCGTGCTTTCAGCTTCGATGTAGGAGGGCGTGGATATGTCCTGCGGGTAAATAG | |
| | CTGCGCCGATGGTTTCTACAAAGATCGTTATGTTTATCGGCACTTTGCATCGGCCGCGCT | |
| | CCCGATTCCGGAAGTGCTTGACATTGGGGAATTCAGCGAGAGCCTGACCTATTGCATCTC | |
| | CCGCCGTGCACAGGGTGTCACGTTGCAAGACCTGCCTGAAACCGAACTGCCCGCTGTTCT | |
| | GCAGCCGGTCGCGGAGGCCATGGATGCGATCGCTGCGGCCGATCTTAGCCAGACGAGCGG | |
| | GTTCGGCCCATTCGGACCGCAAGGAATCGGTCAATACACTACATGGCGTGATTTCATATG | |
| | CGCGATTGCTGATCCCCATGTGTATCACTGGCAAACTGTGATGGACGACACCGTCAGTGC | |
| | GTCCGTCGCGCAGGCTCTCGATGAGCTGATGCTTTGGGCCGAGGACTGCCCCGAAGTCCG | |
| | GCACCTCGTGCACGCGGATTTCGGCTCCAACAATGTCCTGACGGACAATGGCCGCATAAC | |
| | AGCGGTCATTGACTGGAGCGAGGCGATGTTCGGGGATTCCCAATACGAGGTCGCCAACAT | |
| | CTTCTTCTGGAGGCCGTGGTTGGCTTGTATGGAGCAGCAGACGCGCTACTTCGAGCGGAG | |
| | GCATCCGGAGCTTGCAGGATCGCCGCGGCTCCGGGCGTATATGCTCCGCATTGGTCTTGA | |
| | CCAACTCTATCAGAGCTTGGTTGACGGCAATTTCGATGATGCAGCTTGGGCGCAGGGTCG | |
| | ATGCGACGCAATCGTCCGATCCGGAGCCGGGACTGTCGGGCGTACACAAATCGCCCGCAG | |
| | AAGCGCGGCCGTCTGGACCGATGGCTGTGTAGAAGTACTCGCCGATAGTGGAAACCGACG | |
| | CCCCAGCACTCGTCCGAGGGCAAAGGAATAGGCGGGACTCTGGGGTTCGAAATGACCGAC | |
| | CAAGCGACGCCCAACCTGCCATCACGAGATTTCGATTCCACCGCCGCCTTCTATGAAAGG | |
| | TTGGGCTTCGGAATCGTTTTCCGGGACGCCGGCTGGATGATCCTCCAGCGCGGGGATCTC | |
| | ATGCTGGAGTTCTTCGCCCACGGGATCTCTGCGGAACAGGCGGTCGAAGGTGCCGATATC | |
| | ATTACGACAGCAACGGCCGACAAGCACAACGCCACGATCCTGAGCGACAATATGATCGGG | |
| | CCCCGATCGTTCAAACATTTGGCAATAAAGTTTCTTAAGATTGAATCCTGTTGCCGGTCT | |
| | TGCGATGATTATCATATAATTTCTGTTGAATTACGTTAAGCATGTAATAATTAACATGTA | |
| | ATGCATGACGTTATTTATGAGATGGGTTTTTATGATTAGAGTCCCGCAATTATACATTTA | |
| | ATACGCGATAGAAAACAAAATATAGCGCGCAAACTAGGATAAATTATCGCGCGCGGTGTC | |
| | ATCTATGTTACTAGATCtcttccgcttcctcgctcactgactcgctgcgctcggtcgttc | |
| | ggctgcggcgagcggtatcagctcactcaaaggcggtaatacggttatccacagaatcag | |
| | gggataacgcaggaaagaacatgtgagcaaaaggccagcaaaaggccaggaaccgtaaaa | |
| | aggccgcgttgctggcgtttttccataggctccgcccccctgacgagcatcacaaaaatc | |
| | gacgctcaagtcagaggtggcgaaacccgacaggactataaagataccaggcgtttcccc | |
| | ctggaagctccctcgtgcgctctcctgttccgaccctgccgcttaccggatacctgtccg | |
| | cctttctcccttcgggaagcgtggcgctttctcatagctcacgctgtaggtatctcagtt | |
| | cggtgtaggtcgttcgctccaagctgggctgtgtgcacgaaccccccgttcagcccgacc | |
| | gctgcgccttatccggtaactatcgtcttgagtccaacccggtaagacacgacttatcgc | |
| | cactggcagcagccactggtaacaggattagcagagcgaggtatgtaggcggtgctacag | |
| | agttcttgaagtggtggcctaactacggctacactagaagaacagtatttggtatctgcg | |
| | ctctgctgaagccagttaccttcggaaaaagagttggtagctcttgatccggcaaacaaa | |
| | ccaccgctggtagcggtggtttttttgtttgcaagcagcagattacgcgcagaaaaaaag | |
| | gatctcaagaagatcctttgatcttttctacggggtctgacgctcagtggaacgaaaact | |
| | cacgttaagggattttggtcatgagattatcaaaaaggatcttcacctagatccttttaa | |
| | attaaaaatgaagttttaaatcaatctaaagtatatatgagtaaacttggtctgacagtt | |
| | accaatgcttaatcagtgaggcacctatctcagcgatctgtctatttcgttcatccatag | |
| | ttgcctgactccccgtcgtgtagataactacgatacgggagggcttaccatctggcccca | |
| | gtgctgcaatgataccgcgagacccacgctcaccggctccagatttatcagcaataaacc | |
| | agccagccggaagggccgagcgcagaagtggtcctgcaactttatccgcctccatccagt | |
| | ctattaattgttgccgggaagctagagtaagtagttcgccagttaatagtttgcgcaacg | |
| | ttgttgccattgctacaggcatcgtggtgtcacgctcgtcgtttggtatggcttcattca | |
| | gctccggttcccaacgatcaaggcgagttacatgatcccccatgttgtgcaaaaaagcgg | |
| | ttagctccttcggtcctccgatcgttgtcagaagtaagttggccgcagtgttatcactca | |
| | tggttatggcagcactgcataattctcttactgtcatgccatccgtaagatgcttttctg | |
| | tgactggtgagtactcaaccaagtcattctgagaatagtgtatgcggcgaccgagttgct | |
| | cttgcccggcgtcaatacgggataataccgcgccacatagcagaactttaaaagtgctca | |
| | tcattggaaaacgttcttcggggcgaaaactctcaaggatcttaccgctgttgagatcca | |
| | gttcgatgtaacccactcgtgcacccaactgatcttcagcatcttttactttcaccagcg | |
| | tttctgggtgagcaaaaacaggaaggcaaaatgccgcaaaaaagggaataagggcgacac | |
| | ggaaatgttgaatactcatactcttcctttttcaatattattgaagcatttatcagggtt | |
| | attgtctcatgagcggatacatatttgaatgtatttagaaaaataaacaaataggggttc | |
| | cgcgcacatttccccgaaaagtgccacctgacgtctaagaaaccattattatcatgacat | |
| | taacctataaaaataggcgtatcacgaggccctttcgtctcgcgcgtttcggtgatgacg | |
| | gtgaaaacctctgacacatgcagctcccggagacggtcacagcttgtctgtaagcggatg | |
| | ccgggagcagacaagcccgtcagggcgcgtcagcgggtgttggcgggtgtcgggggg | |
| | ttaa | |
| pUC | AAGCTTGCATGCCTGCAGGTCCCCAGATTAGCCTTTTCAATTTCAGAAAGAATGCTAACC | 50 |
| | CACAGATGGTTAGAGAGGCTTACGCAGCAGGTCTCATCAAGACGATCTACCCGAGCAATA | |
| | ATCTCCAGGAAATCAAATACCTTCCCAAGAAGGTTAAAGATGCAGTCAAAAGATTCAGGA | |
| | CTAACTGCATCAAGAACACAGAGAAAGATATATTTCTCAAGATCAGAAGTACTATTCCAG | |
| | TATGGACGATTCAAGGCTTGCTTCACAAACCAAGGCAAGTAATAGAGATTGGAGTCTCTA | |
| | AAAAGGTAGTTCCCACTGAATCAAAGGCCATGGAGTCAAAGATTCAAATAGAGGACCTAA | |
| | CAGAACTCGCCGTAAAGACTGGCGAACAGTTCATACAGAGTCTCTTACGACTCAATGACA | |
| | AGAAGAAAATCTTCGTCAACATGGTGGAGCACGACACACTTGTCTACTCCAAAAATATCA | |
| | AAGATACAGTCTCAGAAGACCAAAGGGCAATTGAGACTTTTCAACAAAGGGTAATATCCG | |
| | GAAACCTCCTCGGATTCCATTGCCCAGCTATCTGTCACTTTATTGTGAAGATAGTGGAAA | |
| | AGGAAGGTGGCTCCTACAAATGCCATCATTGCGATAAAGGAAAGGCCATCGTTGAAGATG | |
| | CCTCTGCCGACAGTGGTCCCAAAGATGGACCCCCACCCACGAGGAGCATCGTGGAAAAAG | |
| | AAGACGTTCCAACCACGTCTTCAAAGCAAGTGGATTGATGTGATATCTCCACTGACGTAA | |
| | GGGATGACGCACAATCCCACTATCCTTCGCAAGACCCTTCCTCTATATAAGGAAGTTCAT | |
| | TTCATTTGGAGAGAACACGGGGGACTCTAGATACATCACAATCACACAAAACTAACAAAA | |
| | GATCAAAAGCAAGTTCTTCACTGTTGATACATATGCCCGTCGATTAGCAAGTTACTACCG | |
| | GGTCGACAATTCTGAATCAACAACTCTCCTGGCGCACCATCGTCGGCTACAGCCTCGGGA | |
| | ATTGCTACCGAGCTCTTATGAAGATGAAGATGAAATATTTGGTETGTCAAATAAAAAGCT | |
| | AGCTTGTGTGCTTAAGTTTGTGTTTTTTTCTTGGCTTGTTGTGTTATGAATTTGTGGCTT | |
| | TTTCTAATATTAAATGAATGTAAGATCTCATTATAATGAATAAACAAATGTTTCTATAAT | |
| | CCATTGTGAATGTTTTGTTGGATCTCTTCGCATATAACTACTGTATGTGCTATGGTATGG | |
| | ACTATGGAATATGATTAAAGATAAGGAATTGGTACCTGCGGCCGCTGGATCCCGGGAATT | |
| | CGTAATCATGGTCATAGCTGTTTCCTGTGTGAAATTGTTATCCGCTCACAATTCCACACA | |
| | ACATACGAGCCGGAAGCATAAAGTGTAAAGCCTGGGGTGCCTAATGAGTGAGCTAACTCA | |
| | CATTAATTGCGTTGCGCTCACTGCCCGCTTTCCAGTCGGGAAACCTGTCGTGCCAGCTGC | |
| | ATTAATGAATCGGCCAACGCGCGGGGAGAGGCGGTTTGCGTATTGGgcgctcttccgctt | |
| | cctcgctcactgactcgctgcgctcggtcgttcggctgcggcgagcggtatcagctcact | |
| | caaaggcggtaatacggttatccacagaatcaggggataacgcaggaaagaacatgtgag | |
| | caaaaggccagcaaaaggccaggaaccgtaaaaaggccgcgttgctggcgtttttccata | |
| | ggctccgcccccctgacgagcatcacaaaaatcgacgctcaagtcagaggtggcgaaacc | |
| | cgacaggactataaagataccaggcgtttccccctggaagctccctcgtgcgctctcctg | |
| | ttccgaccctgccgcttaccggatacctgtccgcctttctcccttcgggaagcgtggcgc | |
| | tttctcatagctcacgctgtaggtatctcagttcggtgtaggtcgttcgctccaagctgg | |
| | gctgtgtgcacgaaccccccgttcagcccgaccgctgcgccttatccggtaactatcgtc | |
| | ttgagtccaacccggtaagacacgacttatcgccactggcagcagccactggtaacagga | |
| | ttagcagagcgaggtatgtaggcggtgctacagagttcttgaagtggtggcctaactacg | |
| | gctacactagaagaacagtatttggtatctgcgctctgctgaagccagttaccttcggaa | |
| | aaagagttggtagctcttgatccggcaaacaaaccaccgctggtagcggtggtttttttg | |
| | tttgcaagcagcagattacgcgcagaaaaaaaggatctcaagaagatcctttgatctttt | |
| | ctacggggtctgacgctcagtggaacgaaaactcacgttaagggattttggtcatgagat | |
| | tatcaaaaaggatcttcacctagatccttttaaattaaaaatgaagttttaaatcaatct | |
| | aaagtatatatgagtaaacttggtctgacagttaccaatgcttaatcagtgaggcaccta | |
| | tctcagcgatctgtctatttcgttcatccatagttgcctgactccccgtcgtgtagataa | |
| | ctacgatacgggagggcttaccatctggccccagtgctgcaatgataccgcgagacccac | |
| | gctcaccggctccagatttatcagcaataaaccagccagccggaagggccgagcgcagaa | |
| | gtggtcctgcaactttatccgcctccatccagtctattaattgttgccgggaagctagag | |
| | taagtagttcgccagttaatagtttgcgcaacgttgttgccattgctacaggcatcgtgg | |
| | tgtcacgctcgtcgtttggtatggcttcattcagctccggttcccaacgatcaaggcgag | |
| | ttacatgatcccccatgttgtgcaaaaaagcggttagctccttcggtcctccgatcgttg | |
| | tcagaagtaagttggccgcagtgttatcactcatggttatggcagcactgcataattctc | |
| | ttactgtcatgccatccgtaagatgcttttctgtgactggtgagtactcaaccaagtcat | |
| | tctgagaatagtgtatgcggcgaccgagttgctcttgcccggcgtcaatacgggataata | |
| | ccgcgccacatagcagaactttaaaagtgctcatcattggaaaacgttcttcggggcgaa | |
| | aactctcaaggatcttaccgctgttgagatccagttcgatgtaacccactcgtgcaccca | |
| | actgatcttcagcatcttttactttcaccagcgtttctgggtgagcaaaaacaggaaggc | |
| | aaaatgccgcaaaaaagggaataagggcgacacggaaatgttgaatactcatactcttcc | |
| | tttttcaatattattgaagcatttatcagggttattgtctcatgagcggatacatatttg | |
| | aatgtatttagaaaaataaacaaataggggttccgcgcacatttccccgaaaagtgccac | |
| | ctgacgtctaagaaaccattattatcatgacattaacctataaaaataggcgtatcacga | |
| | ggccctttcgtctcgcgcgtttcggtgatgacggtgaaaacctctgacacatgcagctcc | |
| | cggagacggtcacagcttgtctgtaagcggatgccgggagcagacaagcccgtcagggcg | |
| | cgtcagcgggtgttggcgggtgtcggggctggcttaa | |
| pUC-GFP- | AAGCTTGCATGCCTGCAGGTCCCCAGATTAGCCTTTTCAATTTCAGAAAGAATGCTAACC | 51 |
| HygroR | CACAGATGGTTAGAGAGGCTTACGCAGCAGGTCTCATCAAGACGATCTACCCGAGCAATA | |
| | ATCTCCAGGAAATCAAATACCTTCCCAAGAAGGTTAAAGATGCAGTCAAAAGATTCAGGA | |
| | CTAACTGCATCAAGAACACAGAGAAAGATATATTTCTCAAGATCAGAAGTACTATTCCAG | |
| | TATGGACGATTCAAGGCTTGCTTCACAAACCAAGGCAAGTAATAGAGATTGGAGTCTCTA | |
| | AAAAGGTAGTTCCCACTGAATCAAAGGCCATGGAGTCAAAGATTCAAATAGAGGACCTAA | |
| | CAGAACTCGCCGTAAAGACTGGCGAACAGTTCATACAGAGTCTCTTACGACTCAATGACA | |
| | AGAAGAAAATCTTCGTCAACATGGTGGAGCACGACACACTTGTCTACTCCAAAAATATCA | |
| | AAGATACAGTCTCAGAAGACCAAAGGGCAATTGAGACTTTTCAACAAAGGGTAATATCCG | |
| | GAAACCTCCTCGGATTCCATTGCCCAGCTATCTGTCACTTTATTGTGAAGATAGTGGAAA | |
| | AGGAAGGTGGCTCCTACAAATGCCATCATTGCGATAAAGGAAAGGCCATCGTTGAAGATG | |
| | CCTCTGCCGACAGTGGTCCCAAAGATGGACCCCCACCCACGAGGAGCATCGTGGAAAAAG | |
| | AAGACGTTCCAACCACGTCTTCAAAGCAAGTGGATTGATGTGATATCTCCACTGACGTAA | |
| | GGGATGACGCACAATCCCACTATCCTTCGCAAGACCCTTCCTCTATATAAGGAAGTTCAT | |
| | TTCATTTGGAGAGAACACGGGGGACTCTAGATACATCACAATCACACAAAACTAACAAAA | |
| | GATCAAAAGCAAGTTCTTCACTGTTGATACATatggtgagcaagggcgaggagctgttca | |
| | ccggggtggtgcccatcctggtcgagctggacggcgacgtaaacggccacaagttcagcg | |
| | tgtctggcgagggcgagggcgatgccacctacggcaagctgaccctgaagttcatctgca | |
| | ccaccggcaagctgcccgtgccctggcccaccctcgtgaccaccctgacctacggcgtgc | |
| | agtgcttcagccgctaccccgaccacatgaagcagcacgacttcttcaagtccgccatgc | |
| | ccgaaggctacgtccaggagcgcaccatcttcttcaaggacgacggcaactacaagaccc | |
| | gcgccgaggtgaagttcgagggcgacaccctggtgaaccgcatcgagctgaagggcatcg | |
| | acttcaaggaggacggcaacatcctggggcacaagctggagtacaactacaacagccaca | |
| | acgtctatatcatggccgacaagcagaagaacggcatcaaggcgaacttcaagatccgcc | |
| | acaacatcgaggacggcagcgtgcagctcgccgaccactaccagcagaacacccccatcg | |
| | gcgacggccccgtgctgctgcccgacaaccactacctgagcacccagtccgccctgagca | |
| | aagaccccaacgagaagcgcgatcacatggtcctgctggagttcgtgaccgccgccggga | |
| | tcactctcggcatggacgagctgtacaagtaaGTCGACAATTCTGAATCAACAACTCTCC | |
| | TGGCGCACCATCGTCGGCTACAGCCTCGGGAATTGCTACCGAGCTCTTATGAAGATGAAG | |
| | ATGAAATATTTGGTGTGTCAAATAAAAAGCTAGCTTGTGTGCTTAAGTTTGTGTTTTTTT | |
| | CTTGGCTTGTTGTGTTATGAATTTGTGGCTTTTTCTAATATTAAATGAATGTAAGATCTC | |
| | ATTATAATGAATAAACAAATGTTTCTATAATCCATTGTGAATGTTTTGTTGGATCTCTTC | |
| | GCATATAACTACTGTATGTGCTATGGTATGGACTATGGAATATGATTAAAGATAAGGAAT | |
| | TGGTACCTGCGGCCGCTGGATCCCGGGAATTCGTAATCATGGTCATAGCTGTTTCCTGTG | |
| | TGAAATTGTTATCCGCTCACAATTCCACACAACATACGAGCCGGAAGCATAAAGTGTAAA | |
| | GCCTGGGGTGCCTAATGAGTGAGCTAACTCACATTAATTGCGTTGCGCTCACTGCCCGCT | |
| | TTCCAGTCGGGAAACCTGTCGTGCCAGCTGCATTAATGAATCGGCCAACGCGCGGGGAGA | |
| | GGCGGTTTGCGTATTGGCTAGAGCAGCTTGCCAACATGGTGGAGCACGACACTCTCGTCT | |
| | ACTCCAAGAATATCAAAGATACAGTCTCAGAAGACCAAAGGGCTATTGAGACTTTTCAAC | |
| | AAAGGGTAATATCGGGAAACCTCCTCGGATTCCATTGCCCAGCTATCTGTCACTTCATCA | |
| | AAAGGACAGTAGAAAAGGAAGGTGGCACCTACAAATGCCATCATTGCGATAAAGGAAAGG | |
| | CTATCGTTCAAGATGCCTCTGCCGACAGTGGTCCCAAAGATGGACCCCCACCCACGAGGA | |
| | GCATCGTGGAAAAAGAAGACGTTCCAACCACGTCTTCAAAGCAAGTGGATTGATGTGATA | |
| | TCTCCACTGACGTAAGGGATGACGCACAATCCCACTATCCTTCGCAAGACCTTCCTCTAT | |
| | ATAAGGAAGTTCATTTCATTTGGAGAGGACACGCTGGAATTCTAGTATACTAAACCATGA | |
| | AAAAGCCTGAACTCACCGCGACGTCTGTCGAGAAGTTTCTGATCGAAAAGTTCGACAGCG | |
| | TCTCCGACCTGATGCAGCTCTCGGAGGGCGAAGAATCTCGTGCTTTCAGCTTCGATGTAG | |
| | GAGGGCGTGGATATGTCCTGCGGGTAAATAGCTGCGCCGATGGTTTCTACAAAGATCGTT | |
| | ATGTTTATCGGCACTTTGCATCGGCCGCGCTCCCGATTCCGGAAGTGCTTGACATTGGGG | |
| | AATTCAGCGAGAGCCTGACCTATTGCATCTCCCGCCGTGCACAGGGTGTCACGTTGCAAG | |
| | ACCTGCCTGAAACCGAACTGCCCGCTGTTCTGCAGCCGGTCGCGGAGGCCATGGATGCGA | |
| | TCGCTGCGGCCGATCTTAGCCAGACGAGCGGGTTCGGCCCATTCGGACCGCAAGGAATCG | |
| | GTCAATACACTACATGGCGTGATTTCATATGCGCGATTGCTGATCCCCATGTGTATCACT | |
| | GGCAAACTGTGATGGACGACACCGTCAGTGCGTCCGTCGCGCAGGCTCTCGATGAGCTGA | |
| | TGCTTTGGGCCGAGGACTGCCCCGAAGTCCGGCACCTCGTGCACGCGGATTTCGGCTCCA | |
| | ACAATGTCCTGACGGACAATGGCCGCATAACAGCGGTCATTGACTGGAGCGAGGCGATGT | |
| | TCGGGGATTCCCAATACGAGGTCGCCAACATCTTCTTCTGGAGGCCGTGGTTGGCTTGTA | |
| | TGGAGCAGCAGACGCGCTACTTCGAGCGGAGGCATCCGGAGCTTGCAGGATCGCCGCGGC | |
| | TCCGGGCGTATATGCTCCGCATTGGTCTTGACCAACTCTATCAGAGCTTGGTTGACGGCA | |
| | ATTTCGATGATGCAGCTTGGGCGCAGGGTCGATGCGACGCAATCGTCCGATCCGGAGCCG | |
| | GGACTGTCGGGCGTACACAAATCGCCCGCAGAAGCGCGGCCGTCTGGACCGATGGCTGTG | |
| | TAGAAGTACTCGCCGATAGTGGAAACCGACGCCCCAGCACTCGTCCGAGGGCAAAGGAAT | |
| | AGGCGGGACTCTGGGGTTCGAAATGACCGACCAAGCGACGCCCAACCTGCCATCACGAGA | |
| | TTTCGATTCCACCGCCGCCTTCTATGAAAGGTTGGGCTTCGGAATCGTTTTCCGGGACGC | |
| | CGGCTGGATGATCCTCCAGCGCGGGGATCTCATGCTGGAGTTCTTCGCCCACGGGATCTC | |
| | TGCGGAACAGGCGGTCGAAGGTGCCGATATCATTACGACAGCAACGGCCGACAAGCACAA | |
| | CGCCACGATCCTGAGCGACAATATGATCGGGCCCCGATCGTTCAAACATTTGGCAATAAA | |
| | GTTTCTTAAGATTGAATCCTGTTGCCGGTCTTGCGATGATTATCATATAATTTCTGTTGA | |
| | ATTACGTTAAGCATGTAATAATTAACATGTAATGCATGACGTTATTTATGAGATGGGTTT | |
| | TTATGATTAGAGTCCCGCAATTATACATTTAATACGCGATAGAAAACAAAATATAGCGCG | |
| | CAAACTAGGATAAATTATCGCGCGCGGTGTCATCTATGTTACTAGATCtcttccgcttcc | |
| | tcgctcactgactcgctgcgctcggtcgttcggctgcggcgagcggtatcagctcactca | |
| | aaggcggtaatacggttatccacagaatcaggggataacgcaggaaagaacatgtgagca | |
| | aaaggccagcaaaaggccaggaaccgtaaaaaggccgcgttgctggcgtttttccatagg | |
| | ctccgcccccctgacgagcatcacaaaaatcgacgctcaagtcagaggtggcgaaacccg | |
| | acaggactataaagataccaggcgtttccccctggaagctccctcgtgcgctctcctgtt | |
| | ccgaccctgccgcttaccggatacctgtccgcctttctcccttcgggaagcgtggcgctt | |
| | tctcatagctcacgctgtaggtatctcagttcggtgtaggtcgttcgctccaagctgggc | |
| | tgtgtgcacgaaccccccgttcagcccgaccgctgcgccttatccggtaactatcgtctt | |
| | gagtccaacccggtaagacacgacttatcgccactggcagcagccactggtaacaggatt | |
| | agcagagcgaggtatgtaggcggtgctacagagttcttgaagtggtggcctaactacggc | |
| | tacactagaagaacagtatttggtatctgcgctctgctgaagccagttaccttcggaaaa | |
| | agagttggtagctcttgatccggcaaacaaaccaccgctggtagcggtggtttttttgtt | |
| | tgcaagcagcagattacgcgcagaaaaaaaggatctcaagaagatcctttgatcttttct | |
| | acggggtctgacgctcagtggaacgaaaactcacgttaagggattttggtcatgagatta | |
| | tcaaaaaggatcttcacctagatccttttaaattaaaaatgaagttttaaatcaatctaa | |
| | agtatatatgagtaaacttggtctgacagttaccaatgcttaatcagtgaggcacctatc | |
| | tcagcgatctgtctatttcgttcatccatagttgcctgactccccgtcgtgtagataact | |
| | acgatacgggagggcttaccatctggccccagtgctgcaatgataccgcgagacccacgc | |
| | tcaccggctccagatttatcagcaataaaccagccagccggaagggccgagcgcagaagt | |
| | ggtcctgcaactttatccgcctccatccagtctattaattgttgccgggaagctagagta | |
| | agtagttcgccagttaatagtttgcgcaacgttgttgccattgctacaggcatcgtggtg | |
| | tcacgctcgtcgtttggtatggcttcattcagctccggttcccaacgatcaaggcgagtt | |
| | acatgatcccccatgttgtgcaaaaaagcggttagctccttcggtcctccgatcgttgtc | |
| | agaagtaagttggccgcagtgttatcactcatggttatggcagcactgcataattctctt | |
| | actgtcatgccatccgtaagatgcttttctgtgactggtgagtactcaaccaagtcattc | |
| | tgagaatagtgtatgcggcgaccgagttgctcttgcccggcgtcaatacgggataatacc | |
| | gcgccacatagcagaactttaaaagtgctcatcattggaaaacgttcttcggggcgaaaa | |
| | ctctcaaggatcttaccgctgttgagatccagttcgatgtaacccactcgtgcacccaac | |
| | tgatcttcagcatcttttactttcaccagcgtttctgggtgagcaaaaacaggaaggcaa | |
| | aatgccgcaaaaaagggaataagggcgacacggaaatgttgaatactcatactcttcctt | |
| | tttcaatattattgaagcatttatcagggttattgtctcatgagcggatacatatttgaa | |
| | tgtatttagaaaaataaacaaataggggttccgcgcacatttccccgaaaagtgccacct | |
| | gacgtctaagaaaccattattatcatgacattaacctataaaaataggcgtatcacgagg | |
| | ccctttcgtctcgcgcgtttcggtgatgacggtgaaaacctctgacacatgcagctcccg | |
| | gagacggtcacagcttgtctgtaagcggatgccgggagcagacaagcccgtcagggcgcg | |
| | tcagcgggtgttggcgggtgtcggggctggcttaa | |
| pUC-GFP | AAGCTTGCATGCCTGCAGGTCCCCAGATTAGCCTTTTCAATTTCAGAAAGAATGCTAACC | 52 |
| | CACAGATGGTTAGAGAGGCTTACGCAGCAGGTCTCATCAAGACGATCTACCCGAGCAATA | |
| | ATCTCCAGGAAATCAAATACCTTCCCAAGAAGGTTAAAGATGCAGTCAAAAGATTCAGGA | |
| | CTAACTGCATCAAGAACACAGAGAAAGATATATTTCTCAAGATCAGAAGTACTATTCCAG | |
| | TATGGACGATTCAAGGCTTGCTTCACAAACCAAGGCAAGTAATAGAGATTGGAGTCTCTA | |
| | AAAAGGTAGTTCCCACTGAATCAAAGGCCATGGAGTCAAAGATTCAAATAGAGGACCTAA | |
| | CAGAACTCGCCGTAAAGACTGGCGAACAGTTCATACAGAGTCTCTTACGACTCAATGACA | |
| | AGAAGAAAATCTTCGTCAACATGGTGGAGCACGACACACTTGTCTACTCCAAAAATATCA | |
| | AAGATACAGTCTCAGAAGACCAAAGGGCAATTGAGACTTTTCAACAAAGGGTAATATCCG | |
| | GAAACCTCCTCGGATTCCATTGCCCAGCTATCTGTCACTTTATTGTGAAGATAGTGGAAA | |
| | AGGAAGGTGGCTCCTACAAATGCCATCATTGCGATAAAGGAAAGGCCATCGTTGAAGATG | |
| | CCTCTGCCGACAGTGGTCCCAAAGATGGACCCCCACCCACGAGGAGCATCGTGGAAAAAG | |
| | AAGACGTTCCAACCACGTCTTCAAAGCAAGTGGATTGATGTGATATCTCCACTGACGTAA | |
| | GGGATGACGCACAATCCCACTATCCTTCGCAAGACCCTTCCTCTATATAAGGAAGTTCAT | |
| | TTCATTTGGAGAGAACACGGGGGACTCTAGATACATCACAATCACACAAAACTAACAAAA | |
| | GATCAAAAGCAAGTTCTTCACTGTTGATACATatggtgagcaagggcgaggagctgttca | |
| | ccggggtggtgcccatcctggtcgagctggacggcgacgtaaacggccacaagttcagcg | |
| | tgtctggcgagggcgagggcgatgccacctacggcaagctgaccctgaagttcatctgca | |
| | ccaccggcaagctgcccgtgccctggcccaccctcgtgaccaccctgacctacggcgtgc | |
| | agtgcttcagccgctaccccgaccacatgaagcagcacgacttcttcaagtccgccatgc | |
| | ccgaaggctacgtccaggagcgcaccatcttcttcaaggacgacggcaactacaagaccc | |
| | gcgccgaggtgaagttcgagggcgacaccctggtgaaccgcatcgagctgaagggcatcg | |
| | acttcaaggaggacggcaacatcctggggcacaagctggagtacaactacaacagccaca | |
| | acgtctatatcatggccgacaagcagaagaacggcatcaaggcgaacttcaagatccgcc | |
| | acaacatcgaggacggcagcgtgcagctcgccgaccactaccagcagaacacccccatcg | |
| | gcgacggccccgtgctgctgcccgacaaccactacctgagcacccagtccgccctgagca | |
| | aagaccccaacgagaagcgcgatcacatggtcctgctggagttcgtgaccgccgccggga | |
| | tcactctcggcatggacgagctgtacaagtaaGTCGACAATTCTGAATCAACAACTCTCC | |
| | TGGCGCACCATCGTCGGCTACAGCCTCGGGAATTGCTACCGAGCTCTTATGAAGATGAAG | |
| | ATGAAATATTTGGTGTGTCAAATAAAAAGCTAGCTTGTGTGCTTAAGTTTGTGTTTTTTT | |
| | CTTGGCTTGTTGTGTTATGAATTTGTGGCTTTTTCTAATATTAAATGAATGTAAGATCTC | |
| | ATTATAATGAATAAACAAATGTTTCTATAATCCATTGTGAATGTTTTGTTGGATCTCTTC | |
| | GCATATAACTACTGTATGTGCTATGGTATGGACTATGGAATATGATTAAAGATAAGGAAT | |
| | TGGTACCTGCGGCCGCTGGATCCCGGGAATTCGTAATCATGGTCATAGCTGTTTCCTGTG | |
| | TGAAATTGTTATCCGCTCACAATTCCACACAACATACGAGCCGGAAGCATAAAGTGTAAA | |
| | GCCTGGGGTGCCTAATGAGTGAGCTAACTCACATTAATTGCGTTGCGCTCACTGCCCGCT | |
| | TTCCAGTCGGGAAACCTGTCGTGCCAGCTGCATTAATGAATCGGCCAACGCGCGGGGAGA | |
| | GGCGGTTTGCGTATTGGgcgctcttccgcttcctcgctcactgactcgctgcgctcggtc | |
| | gttcggctgcggcgagcggtatcagctcactcaaaggcggtaatacggttatccacagaa | |
| | tcaggggataacgcaggaaagaacatgtgagcaaaaggccagcaaaaggccaggaaccgt | |
| | aaaaaggccgcgttgctggcgtttttccataggctccgcccccctgacgagcatcacaaa | |
| | aatcgacgctcaagtcagaggtggcgaaacccgacaggactataaagataccaggcgttt | |
| | ccccctggaagctccctcgtgcgctctcctgttccgaccctgccgcttaccggatacctg | |
| | tccgcctttctcccttcgggaagcgtggcgctttctcatagctcacgctgtaggtatctc | |
| | agttcggtgtaggtcgttcgctccaagctgggctgtgtgcacgaaccccccgttcagccc | |
| | gaccgctgcgccttatccggtaactatcgtcttgagtccaacccggtaagacacgactta | |
| | tcgccactggcagcagccactggtaacaggattagcagagcgaggtatgtaggcggtgct | |
| | acagagttcttgaagtggtggcctaactacggctacactagaagaacagtatttggtatc | |
| | tgcgctctgctgaagccagttaccttcggaaaaagagttggtagctcttgatccggcaaa | |
| | caaaccaccgctggtagcggtggtttttttgtttgcaagcagcagattacgcgcagaaaa | |
| | aaaggatctcaagaagatcctttgatcttttctacggggtctgacgctcagtggaacgaa | |
| | aactcacgttaagggattttggtcatgagattatcaaaaaggatcttcacctagatcctt | |
| | ttaaattaaaaatgaagttttaaatcaatctaaagtatatatgagtaaacttggtctgac | |
| | agttaccaatgcttaatcagtgaggcacctatctcagcgatctgtctatttcgttcatcc | |
| | atagttgcctgactccccgtcgtgtagataactacgatacgggagggcttaccatctggc | |
| | cccagtgctgcaatgataccgcgagacccacgctcaccggctccagatttatcagcaata | |
| | aaccagccagccggaagggccgagcgcagaagtggtcctgcaactttatccgcctccatc | |
| | cagtctattaattgttgccgggaagctagagtaagtagttcgccagttaatagtttgcgc | |
| | aacgttgttgccattgctacaggcatcgtggtgtcacgctcgtcgtttggtatggcttca | |
| | ttcagctccggttcccaacgatcaaggcgagttacatgatcccccatgttgtgcaaaaaa | |
| | gcggttagctccttcggtcctccgatcgttgtcagaagtaagttggccgcagtgttatca | |
| | ctcatggttatggcagcactgcataattctcttactgtcatgccatccgtaagatgcttt | |
| | tctgtgactggtgagtactcaaccaagtcattctgagaatagtgtatgcggcgaccgagt | |
| | tgctcttgcccggcgtcaatacgggataataccgcgccacatagcagaactttaaaagtg | |
| | ctcatcattggaaaacgttcttcggggcgaaaactctcaaggatcttaccgctgttgaga | |
| | tccagttcgatgtaacccactcgtgcacccaactgatcttcagcatcttttactttcacc | |
| | agcgtttctgggtgagcaaaaacaggaaggcaaaatgccgcaaaaaagggaataagggcg | |
| | acacggaaatgttgaatactcatactcttcctttttcaatattattgaagcatttatcag | |
| | ggttattgtctcatgagcggatacatatttgaatgtatttagaaaaataaacaaataggg | |
| | gttccgcgcacatttccccgaaaagtgccacctgacgtctaagaaaccattattatcatg | |
| | acattaacctataaaaataggcgtatcacgaggccctttcgtctcgcgcgtttcggtgat | |
| | gacggtgaaaacctctgacacatgcagctcccggagacggtcacagcttgtctgtaagcg | |
| | gatgccgggagcagacaagcccgtcagggcgcgtcagcgggtgttggcgggtgtcggggc | |
| | tggcttaa | |
| GFP | atggtgagcaagggcgaggagctgttcaccggggggtgcccatcctggtcgagctggac | 53 |
| | ggcgacgtaaacggccacaagttcagcgtgtctggcgagggcgagggcgatgccacctac | |
| | ggcaagctgaccctgaagttcatctgcaccaccggcaagctgcccgtgccctggcccacc | |
| | ctcgtgaccaccctgacctacggcgtgcagtgcttcagccgctaccccgaccacatgaag | |
| | cagcacgacttcttcaagtccgccatgcccgaaggctacgtccaggagcgcaccatcttc | |
| | ttcaaggacgacggcaactacaagacccgcgccgaggtgaagttcgagggcgacaccctg | |
| | gtgaaccgcatcgagctgaagggcatcgacttcaaggaggacggcaacatcctggggcac | |
| | aagctggagtacaactacaacagccacaacgtctatatcatggccgacaagcagaagaac | |
| | ggcatcaaggcgaacttcaagatccgccacaacatcgaggacggcagcgtgcagctcgcc | |
| | gaccactaccagcagaacacccccatcggcgacggccccgtgctgctgcccgacaaccac | |
| | tacctgagcacccagtccgccctgagcaaagaccccaacgagaagcgcgatcacatggtc | |
| | ctgctggagttcgtgaccgccgccgggatcactctcggcatggacgagctgtacaagtaa | |
| pUc- | AAGCTTGCATGCCTGCAGGTCCCCAGATTAGCCTTTTCAATTTCAGAAAGAATGCTAACC | 54 |
| AtEGL3- | CACAGATGGTTAGAGAGGCTTACGCAGCAGGTCTCATCAAGACGATCTACCCGAGCAATA | |
| HygroR | ATCTCCAGGAAATCAAATACCTTCCCAAGAAGGTTAAAGATGCAGTCAAAAGATTCAGGA | |
| | CTAACTGCATCAAGAACACAGAGAAAGATATATTTCTCAAGATCAGAAGTACTATTCCAG | |
| | TATGGACGATTCAAGGCTTGCTTCACAAACCAAGGCAAGTAATAGAGATTGGAGTCTCTA | |
| | AAAAGGTAGTTCCCACTGAATCAAAGGCCATGGAGTCAAAGATTCAAATAGAGGACCTAA | |
| | CAGAACTCGCCGTAAAGACTGGCGAACAGTTCATACAGAGTCTCTTACGACTCAATGACA | |
| | AGAAGAAAATCTTCGTCAACATGGTGGAGCACGACACACTTGTCTACTCCAAAAATATCA | |
| | AAGATACAGTCTCAGAAGACCAAAGGGCAATTGAGACTTTTCAACAAAGGGTAATATCCG | |
| | GAAACCTCCTCGGATTCCATTGCCCAGCTATCTGTCACTTTATTGTGAAGATAGTGGAAA | |
| | AGGAAGGTGGCTCCTACAAATGCCATCATTGCGATAAAGGAAAGGCCATCGTTGAAGATG | |
| | CCTCTGCCGACAGTGGTCCCAAAGATGGACCCCCACCCACGAGGAGCATCGTGGAAAAAG | |
| | AAGACGTTCCAACCACGTCTTCAAAGCAAGTGGATTGATGTGATATCTCCACTGACGTAA | |
| | GGGATGACGCACAATCCCACTATCCTTCGCAAGACCCTTCCTCTATATAAGGAAGTTCAT | |
| | TTCATTTGGAGAGAACACGGGGGACTCTAGATACATCACAATCACACAAAACTAACAAAA | |
| | GATCAAAAGCAAGTTCTTCACTGTTGATACATatggcaaccggagaaaacagaacggtgc | |
| | cggacaatctaaagaaacagctcgcagtttcagttcgaaacattcaatggagttatggaa | |
| | tcttctggtctgtctctgcttctcaaccaggagtgttggagtggggagatggatattaca | |
| | atggagacataaagacaaggaagacgattcaagcagcagaagtcaaaattgaccagttag | |
| | gtcttgagagaagtgagcagcttagagagctttatgaatctctctccctcgctgaatcct | |
| | cagcttccggtagctctcaggtcactagacgagcttccgccgccgctctctcaccggagg | |
| | acctcaccgacaccgagtggtactacttagtatgcatgtctttcgtcttcaacatcggtg | |
| | aaggaatccccggaggagcgttatccaatggagaaccaatatggctttgtaacgctgaaa | |
| | ccgccgatagcaaagtcttcactcgttctcttctagctaaaagtgcttcgcttcagacag | |
| | tggtttgcttcccgtttcttggaggagtccttgagattggcacgaccgaacatattaaag | |
| | aggacatgaacgtgatacaaagtgttaagacgttgttccttgaagctcctccatatacta | |
| | caatatcgacaagatcagactatcaagaaatttttgatcccttaagtgacgataaataca | |
| | ctccggtgtttataaccgaagcttttccaacaacttctactagcgggtttgagcaagaac | |
| | ctgaggatcatgattcgttcatcaacgatggtggtgcgtctcaggtacaaagctggcagt | |
| | ttgtgggtgaagaaatcagtaactgcattcaccaatcgttaaattcaagcgattgcgttt | |
| | cccaaacgtttgttggaacaaccgggagacttgcttgcgatccaaggaagagtaggattc | |
| | aacggttaggtcagattcaagaacagagtaaccatgtaaatatggacgacgatgttcatt | |
| | accaaggcgtgatatcgacgattttcaaaacaacgcatcagctaatactcggaccgcagt | |
| | ttcagaacttcgataagcggtctagcttcacaaggtggaagcgatcatcatctgtgaaaa | |
| | cattgggagagaaatcgcagaagatgataaagaagatactcttcgaggttcctttgatga | |
| | acaagaaagaagagttgttaccggacacaccagaggaaaccgggaaccatgccttgtccg | |
| | agaagaaacgccgcgagaaattgaatgaacggtttatgacattgagatcaatcattccct | |
| | caattagtaagattgataaagtgtcgattcttgatgatacaattgagtatcttcaagatt | |
| | tacagaaacgggttcaagagttggaatcttgtagagaatctgctgatacagagacacgga | |
| | taacgatgatgaagaggaagaaaccggatgatgaggaggaaagagcatcagcgaattgta | |
| | tgaacagcaaaaggaaggggagtgatgtgaatgtaggagaagatgaaccagctgatatcg | |
| | gttatgctggtctaacggataacttaaggatcagttcattaggtaacgaggtggttattg | |
| | agcttagatgcgcttggagagaagggatattgcttgagataatggatgtgattagtgatc | |
| | tcaacttggattctcactcggttcagtcgtcaaccggagacggtttactctgcttaactg | |
| | tcaattgcaagcataaagggacaaaaatagcaacaacaggaatgatccaagaggcacttc | |
| | aaagggttgcatggatatgttaaGTCGACAATTCTGAATCAACAACTCTCCTGGCGCACC | |
| | ATCGTCGGCTACAGCCTCGGGAATTGCTACCGAGCTCTTATGAAGATGAAGATGAAATAT | |
| | TTGGTGTGTCAAATAAAAAGCTAGCTTGTGTGCTTAAGTTTGTGTTTTTTTCTTGGCTTG | |
| | TTGTGTTATGAATTTGTGGCTTTTTCTAATATTAAATGAATGTAAGATCTCATTATAATG | |
| | AATAAACAAATGTTTCTATAATCCATTGTGAATGTTTTGTTGGATCTCTTCGCATATAAC | |
| | TACTGTATGTGCTATGGTATGGACTATGGAATATGATTAAAGATAAGGAATTGGTACCTG | |
| | CGGCCGCTGGATCCCGGGAATTCGTAATCATGGTCATAGCTGTTTCCTGTGTGAAATTGT | |
| | TATCCGCTCACAATTCCACACAACATACGAGCCGGAAGCATAAAGTGTAAAGCCTGGGGT | |
| | GCCTAATGAGTGAGCTAACTCACATTAATTGCGTTGCGCTCACTGCCCGCTTTCCAGTCG | |
| | GGAAACCTGTCGTGCCAGCTGCATTAATGAATCGGCCAACGCGCGGGGAGAGGCGGTTTG | |
| | CGTATTGGCTAGAGCAGCTTGCCAACATGGTGGAGCACGACACTCTCGTCTACTCCAAGA | |
| | ATATCAAAGATACAGTCTCAGAAGACCAAAGGGCTATTGAGACTTTTCAACAAAGGGTAA | |
| | TATCGGGAAACCTCCTCGGATTCCATTGCCCAGCTATCTGTCACTTCATCAAAAGGACAG | |
| | TAGAAAAGGAAGGTGGCACCTACAAATGCCATCATTGCGATAAAGGAAAGGCTATCGTTC | |
| | AAGATGCCTCTGCCGACAGTGGTCCCAAAGATGGACCCCCACCCACGAGGAGCATCGTGG | |
| | AAAAAGAAGACGTTCCAACCACGTCTTCAAAGCAAGTGGATTGATGTGATATCTCCACTG | |
| | ACGTAAGGGATGACGCACAATCCCACTATCCTTCGCAAGACCTTCCTCTATATAAGGAAG | |
| | TTCATTTCATTTGGAGAGGACACGCTGGAATTCTAGTATACTAAACCATGAAAAAGCCTG | |
| | AACTCACCGCGACGTCTGTCGAGAAGTTTCTGATCGAAAAGTTCGACAGCGTCTCCGACC | |
| | TGATGCAGCTCTCGGAGGGCGAAGAATCTCGTGCTTTCAGCTTCGATGTAGGAGGGCGTG | |
| | GATATGTCCTGCGGGTAAATAGCTGCGCCGATGGTTTCTACAAAGATCGTTATGTTTATC | |
| | GGCACTTTGCATCGGCCGCGCTCCCGATTCCGGAAGTGCTTGACATTGGGGAATTCAGCG | |
| | AGAGCCTGACCTATTGCATCTCCCGCCGTGCACAGGGTGTCACGTTGCAAGACCTGCCTG | |
| | AAACCGAACTGCCCGCTGTTCTGCAGCCGGTCGCGGAGGCCATGGATGCGATCGCTGCGG | |
| | CCGATCTTAGCCAGACGAGCGGGTTCGGCCCATTCGGACCGCAAGGAATCGGTCAATACA | |
| | CTACATGGCGTGATTTCATATGCGCGATTGCTGATCCCCATGTGTATCACTGGCAAACTG | |
| | TGATGGACGACACCGTCAGTGCGTCCGTCGCGCAGGCTCTCGATGAGCTGATGCTTTGGG | |
| | CCGAGGACTGCCCCGAAGTCCGGCACCTCGTGCACGCGGATTTCGGCTCCAACAATGTCC | |
| | TGACGGACAATGGCCGCATAACAGCGGTCATTGACTGGAGCGAGGCGATGTTCGGGGATT | |
| | CCCAATACGAGGTCGCCAACATCTTCTTCTGGAGGCCGTGGTTGGCTTGTATGGAGCAGC | |
| | AGACGCGCTACTTCGAGCGGAGGCATCCGGAGCTTGCAGGATCGCCGCGGCTCCGGGCGT | |
| | ATATGCTCCGCATTGGTCTTGACCAACTCTATCAGAGCTTGGTTGACGGCAATTTCGATG | |
| | ATGCAGCTTGGGCGCAGGGTCGATGCGACGCAATCGTCCGATCCGGAGCCGGGACTGTCG | |
| | GGCGTACACAAATCGCCCGCAGAAGCGCGGCCGTCTGGACCGATGGCTGTGTAGAAGTAC | |
| | TCGCCGATAGTGGAAACCGACGCCCCAGCACTCGTCCGAGGGCAAAGGAATAGGCGGGAC | |
| | TCTGGGGTTCGAAATGACCGACCAAGCGACGCCCAACCTGCCATCACGAGATTTCGATTC | |
| | CACCGCCGCCTTCTATGAAAGGTTGGGCTTCGGAATCGTTTTCCGGGACGCCGGCTGGAT | |
| | GATCCTCCAGCGCGGGGATCTCATGCTGGAGTTCTTCGCCCACGGGATCTCTGCGGAACA | |
| | GGCGGTCGAAGGTGCCGATATCATTACGACAGCAACGGCCGACAAGCACAACGCCACGAT | |
| | CCTGAGCGACAATATGATCGGGCCCCGATCGTTCAAACATTTGGCAATAAAGTTTCTTAA | |
| | GATTGAATCCTGTTGCCGGTCTTGCGATGATTATCATATAATTTCTGTTGAATTACGTTA | |
| | AGCATGTAATAATTAACATGTAATGCATGACGTTATTTATGAGATGGGTTTTTATGATTA | |
| | GAGTCCCGCAATTATACATTTAATACGCGATAGAAAACAAAATATAGCGCGCAAACTAGG | |
| | ATAAATTATCGCGCGCGGTGTCATCTATGTTACTAGATCtcttccgcttcctcgctcact | |
| | gactcgctgcgctcggtcgttcggctgcggcgagcggtatcagctcactcaaaggcggta | |
| | atacggttatccacagaatcaggggataacgcaggaaagaacatgtgagcaaaaggccag | |
| | caaaaggccaggaaccgtaaaaaggccgcgttgctggcgtttttccataggctccgcccc | |
| | cctgacgagcatcacaaaaatcgacgctcaagtcagaggtggcgaaacccgacaggacta | |
| | taaagataccaggcgtttccccctggaagctccctcgtgcgctctcctgttccgaccctg | |
| | ccgcttaccggatacctgtccgcctttctcccttcgggaagcgtggcgctttctcatagc | |
| | tcacgctgtaggtatctcagttcggtgtaggtcgttcgctccaagctgggctgtgtgcac | |
| | gaaccccccgttcagcccgaccgctgcgccttatccggtaactatcgtcttgagtccaac | |
| | ccggtaagacacgacttatcgccactggcagcagccactggtaacaggattagcagagcg | |
| | aggtatgtaggcggtgctacagagttcttgaagtggtggcctaactacggctacactaga | |
| | agaacagtatttggtatctgcgctctgctgaagccagttaccttcggaaaaagagttggt | |
| | agctcttgatccggcaaacaaaccaccgctggtagcggtggtttttttgtttgcaagcag | |
| | cagattacgcgcagaaaaaaaggatctcaagaagatcctttgatcttttctacggggtct | |
| | gacgctcagtggaacgaaaactcacgttaagggattttggtcatgagattatcaaaaagg | |
| | atcttcacctagatccttttaaattaaaaatgaagttttaaatcaatctaaagtatatat | |
| | gagtaaacttggtctgacagttaccaatgcttaatcagtgaggcacctatctcagcgatc | |
| | tgtctatttcgttcatccatagttgcctgactccccgtcgtgtagataactacgatacgg | |
| | gagggcttaccatctggccccagtgctgcaatgataccgcgagacccacgctcaccggct | |
| | ccagatttatcagcaataaaccagccagccggaagggccgagcgcagaagtggtcctgca | |
| | actttatccgcctccatccagtctattaattgttgccgggaagctagagtaagtagttcg | |
| | ccagttaatagtttgcgcaacgttgttgccattgctacaggcatcgtggtgtcacgctcg | |
| | tcgtttggtatggcttcattcagctccggttcccaacgatcaaggcgagttacatgatcc | |
| | cccatgttgtgcaaaaaagcggttagctccttcggtcctccgatcgttgtcagaagtaag | |
| | ttggccgcagtgttatcactcatggttatggcagcactgcataattctcttactgtcatg | |
| | ccatccgtaagatgcttttctgtgactggtgagtactcaaccaagtcattctgagaatag | |
| | tgtatgcggcgaccgagttgctcttgcccggcgtcaatacgggataataccgcgccacat | |
| | agcagaactttaaaagtgctcatcattggaaaacgttcttcggggcgaaaactctcaagg | |
| | atcttaccgctgttgagatccagttcgatgtaacccactcgtgcacccaactgatcttca | |
| | gcatcttttactttcaccagcgtttctgggtgagcaaaaacaggaaggcaaaatgccgca | |
| | aaaaagggaataagggcgacacggaaatgttgaatactcatactcttcctttttcaatat | |
| | tattgaagcatttatcagggttattgtctcatgagcggatacatatttgaatgtatttag | |
| | aaaaataaacaaataggggttccgcgcacatttccccgaaaagtgccacctgacgtctaa | |
| | gaaaccattattatcatgacattaacctataaaaataggcgtatcacgaggccctttcgt | |
| | ctcgcgcgtttcggtgatgacggtgaaaacctctgacacatgcagctcccggagacggtc | |
| | acagcttgtctgtaagcggatgccgggagcagacaagcccgtcagggcgcgtcagcgggt | |
| | gttggcgggtgtcggggctggtaa | |
| AtEGL3 | atggcaaccggagaaaacagaacggtgccggacaatctaaagaaacagctcgcagtttca | 55 |
| | gttcgaaacattcaatggagttatggaatcttctggtctgtctctgcttctcaaccagga | |
| | gtgttggagtggggagatggatattacaatggagacataaagacaaggaagacgattcaa | |
| | gcagcagaagtcaaaattgaccagttaggtcttgagagaagtgagcagcttagagagctt | |
| | tatgaatctctctccctcgctgaatcctcagcttccggtagctctcaggtcactagacga | |
| | gcttccgccgccgctctctcaccggaggacctcaccgacaccgagtggtactacttagta | |
| | tgcatgtctttcgtcttcaacatcggtgaaggaatccccggaggagcgttatccaatgga | |
| | gaaccaatatggctttgtaacgctgaaaccgccgatagcaaagtcttcactcgttctctt | |
| | ctagctaaaagtgcttcgcttcagacagtggtttgcttcccgtttcttggaggagtcctt | |
| | gagattggcacgaccgaacatattaaagaggacatgaacgtgatacaaagtgttaagacg | |
| | ttgttccttgaagctcctccatatactacaatatcgacaagatcagactatcaagaaatt | |
| | tttgatcccttaagtgacgataaatacactccggtgtttataaccgaagcttttccaaca | |
| | acttctactagcgggtttgagcaagaacctgaggatcatgattcgttcatcaacgatggt | |
| | ggtgcgtctcaggtacaaagctggcagtttgtgggtgaagaaatcagtaactgcattcac | |
| | caatcgttaaattcaagcgattgcgtttcccaaacgtttgttggaacaaccgggagactt | |
| | gcttgcgatccaaggaagagtaggattcaacggttaggtcagattcaagaacagagtaac | |
| | catgtaaatatggacgacgatgttcattaccaaggcgtgatatcgacgattttcaaaaca | |
| | acgcatcagctaatactcggaccgcagtttcagaacttcgataagcggtctagcttcaca | |
| | aggtggaagcgatcatcatctgtgaaaacattgggagagaaatcgcagaagatgataaag | |
| | aagatactcttcgaggttcctttgatgaacaagaaagaagagttgttaccggacacacca | |
| | gaggaaaccgggaaccatgccttgtccgagaagaaacgccgcgagaaattgaatgaacgg | |
| | tttatgacattgagatcaatcattccctcaattagtaagattgataaagtgtcgattctt | |
| | gatgatacaattgagtatcttcaagatttacagaaacgggttcaagagttggaatcttgt | |
| | agagaatctgctgatacagagacacggataacgatgatgaagaggaagaaaccggatgat | |
| | gaggaggaaagagcatcagcgaattgtatgaacagcaaaaggaaggggagtgatgtgaat | |
| | gtaggagaagatgaaccagctgatatcggttatgctggtctaacggataacttaaggatc | |
| | agttcattaggtaacgaggtggttattgagcttagatgcgcttggagagaagggatattg | |
| | cttgagataatggatgtgattagtgatctcaacttggattctcactcggttcagtcgtca | |
| | accggagacggtttactctgcttaactgtcaattgcaagcataaagggacaaaaatagca | |
| | acaacaggaatgatccaagaggcacttcaaagggttgcatggatatgttaa | |
| pUC- | AAGCTTGCATGCCTGCAGGTCCCCAGATTAGCCTTTTCAATTTCAGAAAGAATGCTAACC | 56 |
| Cas12a- | CACAGATGGTTAGAGAGGCTTACGCAGCAGGTCTCATCAAGACGATCTACCCGAGCAATA | |
| HygroR | ATCTCCAGGAAATCAAATACCTTCCCAAGAAGGTTAAAGATGCAGTCAAAAGATTCAGGA | |
| | CTAACTGCATCAAGAACACAGAGAAAGATATATTTCTCAAGATCAGAAGTACTATTCCAG | |
| | TATGGACGATTCAAGGCTTGCTTCACAAACCAAGGCAAGTAATAGAGATTGGAGTCTCTA | |
| | AAAAGGTAGTTCCCACTGAATCAAAGGCCATGGAGTCAAAGATTCAAATAGAGGACCTAA | |
| | CAGAACTCGCCGTAAAGACTGGCGAACAGTTCATACAGAGTCTCTTACGACTCAATGACA | |
| | AGAAGAAAATCTTCGTCAACATGGTGGAGCACGACACACTTGTCTACTCCAAAAATATCA | |
| | AAGATACAGTCTCAGAAGACCAAAGGGCAATTGAGACTTTTCAACAAAGGGTAATATCCG | |
| | GAAACCTCCTCGGATTCCATTGCCCAGCTATCTGTCACTTTATTGTGAAGATAGTGGAAA | |
| | AGGAAGGTGGCTCCTACAAATGCCATCATTGCGATAAAGGAAAGGCCATCGTTGAAGATG | |
| | CCTCTGCCGACAGTGGTCCCAAAGATGGACCCCCACCCACGAGGAGCATCGTGGAAAAAG | |
| | AAGACGTTCCAACCACGTCTTCAAAGCAAGTGGATTGATGTGATATCTCCACTGACGTAA | |
| | GGGATGACGCACAATCCCACTATCCTTCGCAAGACCCTTCCTCTATATAAGGAAGTTCAT | |
| | TTCATTTGGAGAGAACACGGGGGACTCTAGATACATCACAATCACACAAAACTAACAAAA | |
| | GATCAAAAGCAAGTTCTTCACTGTTGATACATATGGCTCCTAAGAAGAAGCGGAAGGTTG | |
| | GTATTCACGGGGTGCCTGCGGCTTCAAAGCTCGAGAAATTCACCAACTGTTATTCGTTGA | |
| | GCAAAACACTGCGGTTTAAAGCGATTCCAGTCGGCAAGACTCAAGAGAATATAGACAATA | |
| | AGCGGCTGTTGGTGGAAGATGAAAAGCGCGCGGAAGACTACAAAGGGGTGAAGAAGTTGT | |
| | TGGACAGATACTACCTCTCTTTTATCAATGATGTCTTGCACTCAATCAAATTGAAGAATC | |
| | TGAACAACTACATCTCCCTCTTCAGAAAGAAAACAAGGACAGAAAAGGAGAATAAGGAAC | |
| | TTGAAAATTTGGAGATCAATCTGAGGAAAGAGATCGCGAAAGCCTTTAAAGGCAACGAAG | |
| | GATACAAAAGTCTGTTCAAGAAGGATATAATTGAGACAATTTTGCCAGAGTTCCTCGATG | |
| | ACAAGGACGAGATTGCGCTGGTCAATTCGTTCAACGGATTCACAACAGCATTCACAGGCT | |
| | TCTTTGATAATCGGGAAAATATGTTCTCTGAGGAGGCAAAGTCCACTTCTATTGCGTTCA | |
| | GGTGTATCAATGAGAATCTCACTAGGTACATTTCCAACATGGATATCTTTGAGAAGGTTG | |
| | ACGCAATTTTTGACAAGCACGAAGTTCAGGAGATTAAGGAGAAGATCCTCAATTCCGATT | |
| | ATGACGTTGAGGACTTCTTCGAAGGTGAGTTTTTTAATTTCGTGCTCACTCAAGAGGGTA | |
| | TCGACGTGTATAATGCGATCATCGGTGGGTTCGTGACTGAGTCCGGTGAAAAGATTAAGG | |
| | GATTGAACGAGTATATCAACCTTTACAACCAAAAGACGAAACAGAAGCTGCCAAAGTTCA | |
| | AGCCTCTTTACAAACAGGTTCTTTCAGACCGCGAGTCACTCTCGTTCTATGGGGAGGGCT | |
| | ACACTTCGGATGAGGAAGTCCTGGAGGTGTTCAGGAATACTCTCAATAAGAATTCGGAGA | |
| | TTTTCTCTTCTATAAAAAAACTGGAAAAGTTGTTTAAGAATTTTGACGAATACTCTAGCG | |
| | CCGGCATATTTGTGAAAAACGGCCCGGCCATATCAACGATAAGTAAAGATATCTTCGGCG | |
| | AATGGAACGTGATCAGAGACAAATGGAACGCGGAGTATGACGATATTCACCTGAAGAAGA | |
| | AGGCTGTCGTAACGGAGAAGTACGAGGATGATCGCAGGAAAAGCTTCAAAAAGATCGGAA | |
| | GTTTCAGCCTGGAACAGTTGCAGGAGTATGCTGACGCCGATCTTAGCGTCGTCGAGAAGT | |
| | TGAAGGAGATAATCATCCAAAAGGTCGACGAGATATATAAAGTCTATGGATCAAGTGAAA | |
| | AACTGTTCGACGCCGACTTCGTTTTGGAGAAGTCCCTGAAGAAGAACGACGCTGTTGTTG | |
| | CCATTATGAAGGATCTGCTCGACAGCGTGAAGAGTTTCGAGAACTATATTAAGGCTTTTT | |
| | TCGGGGAGGGGAAGGAGACTAACAGAGATGAGTCCTTCTACGGAGACTTCGTCCTCGCGT | |
| | ACGATATACTCCTTAAGGTAGACCACATCTACGACGCAATCAGAAATTACGTGACACAAA | |
| | AGCCGTACAGCAAGGACAAGTTCAAACTCTACTTCCAGAACCCCCAGTTCATGGGCGGCT | |
| | GGGACAAGGACAAGGAAACGGATTACAGGGCTACGATCCTGAGGTATGGTTCAAAATACT | |
| | ACTTGGCGATTATGGACAAGAAGTACGCCAAGTGTCTCCAGAAGATTGACAAAGACGATG | |
| | TCAATGGCAATTATGAGAAGATCAACTACAAGCTGCTTCCGGGTCCGAACAAGATGCTCC | |
| | CAAAGGTTTTCTTCAGCAAGAAATGGATGGCCTACTATAACCCAAGCGAGGACATCCAGA | |
| | AGATTTATAAGAACGGTACGTTCAAGAAGGGCGACATGTTCAATCTTAACGACTGTCACA | |
| | AGCTGATCGACTTCTTCAAAGACTCAATTAGCCGGTACCCAAAGTGGTCTAACGCCTATG | |
| | ACTTCAACTTTTCGGAAACCGAGAAGTACAAGGATATAGCCGGATTTTATAGAGAGGTGG | |
| | AAGAGCAGGGCTACAAGGTGTCATTCGAGTCCGCCAGCAAGAAGGAAGTGGACAAGCTCG | |
| | TGGAAGAGGGTAAGCTCTACATGTTCCAGATTTATAATAAAGACTTTAGCGATAAGAGCC | |
| | ACGGGACACCTAATCTCCACACAATGTATTTCAAGCTGCTCTTCGACGAGAATAACCACG | |
| | GCCAAATCAGGTTGTCAGGAGGGGCTGAACTCTTCATGCGGCGCGCTAGCCTTAAGAAGG | |
| | AGGAGCTTGTAGTCCACCCTGCGAATAGTCCAATTGCGAATAAGAACCCGGACAATCCTA | |
| | AAAAGACTACAACATTGAGCTACGACGTGTACAAGGATAAGAGGTTTTCCGAGGATCAGT | |
| | ACGAGCTCCACATCCCGATTGCGATCAACAAGTGCCCAAAGAATATTTTCAAGATAAACA | |
| | CAGAGGTGCGTGTACTCCTGAAGCATGACGACAATCCTTACGTCATTGGGATTGATCGGG | |
| | GCGAGAGGAACCTCCTCTATATTGTGGTGGTGGACGGGAAGGGGAACATAGTCGAACAGT | |
| | ACTCCCTTAACGAAATAATTAACAATTTCAACGGCATCCGTATCAAGACCGACTACCATT | |
| | CGTTGCTGGACAAGAAGGAGAAGGAGAGATTTGAGGCGCGGCAAAATTGGACAAGTATCG | |
| | AGAACATCAAGGAACTCAAAGCAGGTTATATCTCTCAAGTTGTGCATAAGATATGCGAGC | |
| | TGGTTGAGAAGTATGACGCAGTGATCGCTCTTGAGGACCTCAACTCGGGCTTTAAGAATT | |
| | CTAGAGTTAAAGTGGAGAAGCAGGTCTATCAAAAGTTCGAGAAGATGCTTATAGATAAGC | |
| | TCAACTACATGGTCGATAAGAAATCGAACCCATGTGCCACCGGCGGCGCACTCAAAGGTT | |
| | ACCAAATAACAAACAAATTCGAGTCCTTCAAATCGATGAGTACTCAGAATGGGTTCATAT | |
| | TTTATATACCGGCGTGGCTTACGTCTAAGATCGACCCGTCAACTGGTTTTGTCAACCTGT | |
| | TGAAGACGAAATACACGTCCATTGCCGATTCGAAAAAGTTCATATCTAGTTTTGATCGTA | |
| | TTATGTACGTCCCAGAGGAAGATCTTTTCGAGTTTGCTCTCGACTACAAAAACTTTTCGC | |
| | GGACCGATGCGGATTACATTAAAAAATGGAAACTCTATTCGTACGGCAACAGAATCAGGA | |
| | TTTTTCGCAACCCTAAGAAGAATAACGTCTTTGATTGGGAGGAAGTTTGCTTGACTAGCG | |
| | CGTACAAGGAGCTCTTTAATAAGTATGGCATTAACTACCAACAGGGTGATATCAGAGCAC | |
| | TGCTTTGCGAACAATCTGACAAGGCTTTCTACTCATCCTTCATGGCTTTGATGAGCCTGA | |
| | TGCTCCAGATGAGAAATTCAATTACAGGCAGAACCGACGTGGATTTCTTGATCTCCCCGG | |
| | TTAAAAATTCTGATGGCATCTTTTACGATAGCAGGAACTATGAAGCGCAAGAGAATGCGA | |
| | TTCTGCCAAAAAATGCAGACGCCAACGGTGCCTATAACATCGCCAGGAAAGTCCTGTGGG | |
| | CGATCGGCCAGTTCAAAAAGGCCGAAGACGAAAAATTGGACAAGGTCAAAATCGCTATCA | |
| | GCAACAAAGAGTGGCTGGAGTATGCTCAGACATCCGTAAAGCATAAGCGTCCTGCTGCCA | |
| | CCAAAAAGGCCGGACAGGCTAAGAAAAAGAAGTGAGTCGACAATTCTGAATCAACAACTC | |
| | TCCTGGCGCACCATCGTCGGCTACAGCCTCGGGAATTGCTACCGAGCTCTTATGAAGATG | |
| | AAGATGAAATATTTGGTGTGTCAAATAAAAAGCTAGCTTGTGTGCTTAAGTTTGTGTTTT | |
| | TTTCTTGGCTTGTTGTGTTATGAATTTGTGGCTTTTTCTAATATTAAATGAATGTAAGAT | |
| | CTCATTATAATGAATAAACAAATGTTTCTATAATCCATTGTGAATGTTTTGTTGGATCTC | |
| | TTCGCATATAACTACTGTATGTGCTATGGTATGGACTATGGAATATGATTAAAGATAAGG | |
| | AATTGGTACCTGCGGCTTTTTTTCTTCTTCTTCGTTCATACAGTTTTTTTTTGTTTATCA | |
| | GCTTACATTTTCTTGAACCGTAGCTTTCGTTTTCTTCTTTTTAACTTTCCATTCGGAGTT | |
| | TTTGTATCTTGTTTCATAGTTTGTCCCAGGATTAGAATGATTAGGCATCGAACCTTCAAG | |
| | AATTTGATTGAATAAAACATCTTCATTCTTAAGATATGAAGATAATCTTCAAAAGGCCCC | |
| | TGGGAATCTGAAAGAAGAGAAGCAGGCCCATTTATATGGGAAAGAACAATAGTATTTCTT | |
| | ATATAGGCCCATTTAAGTTGAAAACAATCTTCAAAAGTCCCACATCGCTTAGATAAGAAA | |
| | ACGAAGCTGAGTTTATATACAGCTAGAGTCGAAGTAGTGATTGTAATTTCTACTAAGTGT | |
| | AGATnnnnnnnnnnnnnnnnnnnnnGGCCGGCATGGTCCCAGCCTCCTCGCTGGCGCCGG | |
| | CTGGGCAACATGCTTCGGCATGGCGAATGGGACTTTTTTTTCTAGACCCAGCTTTCTTGT | |
| | ACAAAGTTGGCATTACGCTGGATCCCGGGAATTCGTAATCATGGTCATAGCTGTTTCCTG | |
| | TGTGAAATTGTTATCCGCTCACAATTCCACACAACATACGAGCCGGAAGCATAAAGTGTA | |
| | AAGCCTGGGGTGCCTAATGAGTGAGCTAACTCACATTAATTGCGTTGCGCTCACTGCCCG | |
| | CTTTCCAGTCGGGAAACCTGTCGTGCCAGCTGCATTAATGAATCGGCCAACGCGCGGGGA | |
| | GAGGCGGTTTGCGTATTGGCTAGAGCAGCTTGCCAACATGGTGGAGCACGACACTCTCGT | |
| | CTACTCCAAGAATATCAAAGATACAGTCTCAGAAGACCAAAGGGCTATTGAGACTTTTCA | |
| | ACAAAGGGTAATATCGGGAAACCTCCTCGGATTCCATTGCCCAGCTATCTGTCACTTCAT | |
| | CAAAAGGACAGTAGAAAAGGAAGGTGGCACCTACAAATGCCATCATTGCGATAAAGGAAA | |
| | GGCTATCGTTCAAGATGCCTCTGCCGACAGTGGTCCCAAAGATGGACCCCCACCCACGAG | |
| | GAGCATCGTGGAAAAAGAAGACGTTCCAACCACGTCTTCAAAGCAAGTGGATTGATGTGA | |
| | TATCTCCACTGACGTAAGGGATGACGCACAATCCCACTATCCTTCGCAAGACCTTCCTCT | |
| | ATATAAGGAAGTTCATTTCATTTGGAGAGGACACGCTGGAATTCTAGTATACTAAACCAT | |
| | GAAAAAGCCTGAACTCACCGCGACGTCTGTCGAGAAGTTTCTGATCGAAAAGTTCGACAG | |
| | CGTCTCCGACCTGATGCAGCTCTCGGAGGGCGAAGAATCTCGTGCTTTCAGCTTCGATGT | |
| | AGGAGGGCGTGGATATGTCCTGCGGGTAAATAGCTGCGCCGATGGTTTCTACAAAGATCG | |
| | TTATGTTTATCGGCACTTTGCATCGGCCGCGCTCCCGATTCCGGAAGTGCTTGACATTGG | |
| | GGAATTCAGCGAGAGCCTGACCTATTGCATCTCCCGCCGTGCACAGGGTGTCACGTTGCA | |
| | AGACCTGCCTGAAACCGAACTGCCCGCTGTTCTGCAGCCGGTCGCGGAGGCCATGGATGC | |
| | GATCGCTGCGGCCGATCTTAGCCAGACGAGCGGGTTCGGCCCATTCGGACCGCAAGGAAT | |
| | CGGTCAATACACTACATGGCGTGATTTCATATGCGCGATTGCTGATCCCCATGTGTATCA | |
| | CTGGCAAACTGTGATGGACGACACCGTCAGTGCGTCCGTCGCGCAGGCTCTCGATGAGCT | |
| | GATGCTTTGGGCCGAGGACTGCCCCGAAGTCCGGCACCTCGTGCACGCGGATTTCGGCTC | |
| | CAACAATGTCCTGACGGACAATGGCCGCATAACAGCGGTCATTGACTGGAGCGAGGCGAT | |
| | GTTCGGGGATTCCCAATACGAGGTCGCCAACATCTTCTTCTGGAGGCCGTGGTTGGCTTG | |
| | TATGGAGCAGCAGACGCGCTACTTCGAGCGGAGGCATCCGGAGCTTGCAGGATCGCCGCG | |
| | GCTCCGGGCGTATATGCTCCGCATTGGTCTTGACCAACTCTATCAGAGCTTGGTTGACGG | |
| | CAATTTCGATGATGCAGCTTGGGCGCAGGGTCGATGCGACGCAATCGTCCGATCCGGAGC | |
| | CGGGACTGTCGGGCGTACACAAATCGCCCGCAGAAGCGCGGCCGTCTGGACCGATGGCTG | |
| | TGTAGAAGTACTCGCCGATAGTGGAAACCGACGCCCCAGCACTCGTCCGAGGGCAAAGGA | |
| | ATAGGCGGGACTCTGGGGTTCGAAATGACCGACCAAGCGACGCCCAACCTGCCATCACGA | |
| | GATTTCGATTCCACCGCCGCCTTCTATGAAAGGTTGGGCTTCGGAATCGTTTTCCGGGAC | |
| | GCCGGCTGGATGATCCTCCAGCGCGGGGATCTCATGCTGGAGTTCTTCGCCCACGGGATC | |
| | TCTGCGGAACAGGCGGTCGAAGGTGCCGATATCATTACGACAGCAACGGCCGACAAGCAC | |
| | AACGCCACGATCCTGAGCGACAATATGATCGGGCCCCGATCGTTCAAACATTTGGCAATA | |
| | AAGTTTCTTAAGATTGAATCCTGTTGCCGGTCTTGCGATGATTATCATATAATTTCTGTT | |
| | GAATTACGTTAAGCATGTAATAATTAACATGTAATGCATGACGTTATTTATGAGATGGGT | |
| | TTTTATGATTAGAGTCCCGCAATTATACATTTAATACGCGATAGAAAACAAAATATAGCG | |
| | CGCAAACTAGGATAAATTATCGCGCGCGGTGTCATCTATGTTACTAGATCtcttccgctt | |
| | cctcgctcactgactcgctgcgctcggtcgttcggctgcggcgagcggtatcagctcact | |
| | caaaggcggtaatacggttatccacagaatcaggggataacgcaggaaagaacatgtgag | |
| | caaaaggccagcaaaaggccaggaaccgtaaaaaggccgcgttgctggcgtttttccata | |
| | ggctccgcccccctgacgagcatcacaaaaatcgacgctcaagtcagaggtggcgaaacc | |
| | cgacaggactataaagataccaggcgtttccccctggaagctccctcgtgcgctctcctg | |
| | ttccgaccctgccgcttaccggatacctgtccgcctttctcccttcgggaagcgtggcgc | |
| | tttctcatagctcacgctgtaggtatctcagttcggtgtaggtcgttcgctccaagctgg | |
| | gctgtgtgcacgaaccccccgttcagcccgaccgctgcgccttatccggtaactatcgtc | |
| | ttgagtccaacccggtaagacacgacttatcgccactggcagcagccactggtaacagga | |
| | ttagcagagcgaggtatgtaggcggtgctacagagttcttgaagtggtggcctaactacg | |
| | gctacactagaagaacagtatttggtatctgcgctctgctgaagccagttaccttcggaa | |
| | aaagagttggtagctcttgatccggcaaacaaaccaccgctggtagcggtggtttttttg | |
| | tttgcaagcagcagattacgcgcagaaaaaaaggatctcaagaagatcctttgatctttt | |
| | ctacggggtctgacgctcagtggaacgaaaactcacgttaagggattttggtcatgagat | |
| | tatcaaaaaggatcttcacctagatccttttaaattaaaaatgaagttttaaatcaatct | |
| | aaagtatatatgagtaaacttggtctgacagttaccaatgcttaatcagtgaggcaccta | |
| | tctcagcgatctgtctatttcgttcatccatagttgcctgactccccgtcgtgtagataa | |
| | ctacgatacgggagggcttaccatctggccccagtgctgcaatgataccgcgagacccac | |
| | gctcaccggctccagatttatcagcaataaaccagccagccggaagggccgagcgcagaa | |
| | gtggtcctgcaactttatccgcctccatccagtctattaattgttgccgggaagctagag | |
| | taagtagttcgccagttaatagtttgcgcaacgttgttgccattgctacaggcatcgtgg | |
| | tgtcacgctcgtcgtttggtatggcttcattcagctccggttcccaacgatcaaggcgag | |
| | ttacatgatcccccatgttgtgcaaaaaagcggttagctccttcggtcctccgatcgttg | |
| | tcagaagtaagttggccgcagtgttatcactcatggttatggcagcactgcataattctc | |
| | ttactgtcatgccatccgtaagatgcttttctgtgactggtgagtactcaaccaagtcat | |
| | tctgagaatagtgtatgcggcgaccgagttgctcttgcccggcgtcaatacgggataata | |
| | ccgcgccacatagcagaactttaaaagtgctcatcattggaaaacgttcttcggggcgaa | |
| | aactctcaaggatcttaccgctgttgagatccagttcgatgtaacccactcgtgcaccca | |
| | actgatcttcagcatcttttactttcaccagcgtttctgggtgagcaaaaacaggaaggc | |
| | aaaatgccgcaaaaaagggaataagggcgacacggaaatgttgaatactcatactcttcc | |
| | tttttcaatattattgaagcatttatcagggttattgtctcatgagcggatacatatttg | |
| | aatgtatttagaaaaataaacaaataggggttccgcgcacatttccccgaaaagtgccac | |
| | ctgacgtctaagaaaccattattatcatgacattaacctataaaaataggcgtatcacga | |
| | ggccctttcgtctcgcgcgtttcggtgatgacggtgaaaacctctgacacatgcagctcc | |
| | cggagacggtcacagcttgtctgtaagcggatgccgggagcagacaagcccgtcagggcg | |
| | cgtcagcgggtgttggcgggtgtcggggctggcttaa | |
| LbCas12a- | ATGGCTCCTAAGAAGAAGCGGAAGGTTGGTATTCACGGGGTGCCTGCGGCTTCAAAGCTC | 57 |
| NLS | GAGAAATTCACCAACTGTTATTCGTTGAGCAAAACACTGCGGTTTAAAGCGATTCCAGTC | |
| | GGCAAGACTCAAGAGAATATAGACAATAAGCGGCTGTTGGTGGAAGATGAAAAGCGCGCG | |
| | GAAGACTACAAAGGGGTGAAGAAGTTGTTGGACAGATACTACCTCTCTTTTATCAATGAT | |
| | GTCTTGCACTCAATCAAATTGAAGAATCTGAACAACTACATCTCCCTCTTCAGAAAGAAA | |
| | ACAAGGACAGAAAAGGAGAATAAGGAACTTGAAAATTTGGAGATCAATCTGAGGAAAGAG | |
| | ATCGCGAAAGCCTTTAAAGGCAACGAAGGATACAAAAGTCTGTTCAAGAAGGATATAATT | |
| | GAGACAATTTTGCCAGAGTTCCTCGATGACAAGGACGAGATTGCGCTGGTCAATTCGTTC | |
| | AACGGATTCACAACAGCATTCACAGGCTTCTTTGATAATCGGGAAAATATGTTCTCTGAG | |
| | GAGGCAAAGTCCACTTCTATTGCGTTCAGGTGTATCAATGAGAATCTCACTAGGTACATT | |
| | TCCAACATGGATATCTTTGAGAAGGTTGACGCAATTTTTGACAAGCACGAAGTTCAGGAG | |
| | ATTAAGGAGAAGATCCTCAATTCCGATTATGACGTTGAGGACTTCTTCGAAGGTGAGTTT | |
| | TTTAATTTCGTGCTCACTCAAGAGGGTATCGACGTGTATAATGCGATCATCGGTGGGTTC | |
| | GTGACTGAGTCCGGTGAAAAGATTAAGGGATTGAACGAGTATATCAACCTTTACAACCAA | |
| | AAGACGAAACAGAAGCTGCCAAAGTTCAAGCCTCTTTACAAACAGGTTCTTTCAGACCGC | |
| | GAGTCACTCTCGTTCTATGGGGAGGGCTACACTTCGGATGAGGAAGTCCTGGAGGTGTTC | |
| | AGGAATACTCTCAATAAGAATTCGGAGATTTTCTCTTCTATAAAAAAACTGGAAAAGTTG | |
| | TTTAAGAATTTTGACGAATACTCTAGCGCCGGCATATTTGTGAAAAACGGCCCGGCCATA | |
| | TCAACGATAAGTAAAGATATCTTCGGCGAATGGAACGTGATCAGAGACAAATGGAACGCG | |
| | GAGTATGACGATATTCACCTGAAGAAGAAGGCTGTCGTAACGGAGAAGTACGAGGATGAT | |
| | CGCAGGAAAAGCTTCAAAAAGATCGGAAGTTTCAGCCTGGAACAGTTGCAGGAGTATGCT | |
| | GACGCCGATCTTAGCGTCGTCGAGAAGTTGAAGGAGATAATCATCCAAAAGGTCGACGAG | |
| | ATATATAAAGTCTATGGATCAAGTGAAAAACTGTTCGACGCCGACTTCGTTTTGGAGAAG | |
| | TCCCTGAAGAAGAACGACGCTGTTGTTGCCATTATGAAGGATCTGCTCGACAGCGTGAAG | |
| | AGTTTCGAGAACTATATTAAGGCTTTTTTCGGGGAGGGGAAGGAGACTAACAGAGATGAG | |
| | TCCTTCTACGGAGACTTCGTCCTCGCGTACGATATACTCCTTAAGGTAGACCACATCTAC | |
| | GACGCAATCAGAAATTACGTGACACAAAAGCCGTACAGCAAGGACAAGTTCAAACTCTAC | |
| | TTCCAGAACCCCCAGTTCATGGGCGGCTGGGACAAGGACAAGGAAACGGATTACAGGGCT | |
| | ACGATCCTGAGGTATGGTTCAAAATACTACTTGGCGATTATGGACAAGAAGTACGCCAAG | |
| | TGTCTCCAGAAGATTGACAAAGACGATGTCAATGGCAATTATGAGAAGATCAACTACAAG | |
| | CTGCTTCCGGGTCCGAACAAGATGCTCCCAAAGGTTTTCTTCAGCAAGAAATGGATGGCC | |
| | TACTATAACCCAAGCGAGGACATCCAGAAGATTTATAAGAACGGTACGTTCAAGAAGGGC | |
| | GACATGTTCAATCTTAACGACTGTCACAAGCTGATCGACTTCTTCAAAGACTCAATTAGC | |
| | CGGTACCCAAAGTGGTCTAACGCCTATGACTTCAACTTTTCGGAAACCGAGAAGTACAAG | |
| | GATATAGCCGGATTTTATAGAGAGGTGGAAGAGCAGGGCTACAAGGTGTCATTCGAGTCC | |
| | GCCAGCAAGAAGGAAGTGGACAAGCTCGTGGAAGAGGGTAAGCTCTACATGTTCCAGATT | |
| | TATAATAAAGACTTTAGCGATAAGAGCCACGGGACACCTAATCTCCACACAATGTATTTC | |
| | AAGCTGCTCTTCGACGAGAATAACCACGGCCAAATCAGGTTGTCAGGAGGGGCTGAACTC | |
| | TTCATGCGGCGCGCTAGCCTTAAGAAGGAGGAGCTTGTAGTCCACCCTGCGAATAGTCCA | |
| | ATTGCGAATAAGAACCCGGACAATCCTAAAAAGACTACAACATTGAGCTACGACGTGTAC | |
| | AAGGATAAGAGGTTTTCCGAGGATCAGTACGAGCTCCACATCCCGATTGCGATCAACAAG | |
| | TGCCCAAAGAATATTTTCAAGATAAACACAGAGGTGCGTGTACTCCTGAAGCATGACGAC | |
| | AATCCTTACGTCATTGGGATTGATCGGGGCGAGAGGAACCTCCTCTATATTGTGGTGGTG | |
| | GACGGGAAGGGGAACATAGTCGAACAGTACTCCCTTAACGAAATAATTAACAATTTCAAC | |
| | GGCATCCGTATCAAGACCGACTACCATTCGTTGCTGGACAAGAAGGAGAAGGAGAGATTT | |
| | GAGGCGCGGCAAAATTGGACAAGTATCGAGAACATCAAGGAACTCAAAGCAGGTTATATC | |
| | TCTCAAGTTGTGCATAAGATATGCGAGCTGGTTGAGAAGTATGACGCAGTGATCGCTCTT | |
| | GAGGACCTCAACTCGGGCTTTAAGAATTCTAGAGTTAAAGTGGAGAAGCAGGTCTATCAA | |
| | AAGTTCGAGAAGATGCTTATAGATAAGCTCAACTACATGGTCGATAAGAAATCGAACCCA | |
| | TGTGCCACCGGCGGCGCACTCAAAGGTTACCAAATAACAAACAAATTCGAGTCCTTCAAA | |
| | TCGATGAGTACTCAGAATGGGTTCATATTTTATATACCGGCGTGGCTTACGTCTAAGATC | |
| | GACCCGTCAACTGGTTTTGTCAACCTGTTGAAGACGAAATACACGTCCATTGCCGATTCG | |
| | AAAAAGTTCATATCTAGTTTTGATCGTATTATGTACGTCCCAGAGGAAGATCTTTTCGAG | |
| | TTTGCTCTCGACTACAAAAACTTTTCGCGGACCGATGCGGATTACATTAAAAAATGGAAA | |
| | CTCTATTCGTACGGCAACAGAATCAGGATTTTTCGCAACCCTAAGAAGAATAACGTCTTT | |
| | GATTGGGAGGAAGTTTGCTTGACTAGCGCGTACAAGGAGCTCTTTAATAAGTATGGCATT | |
| | AACTACCAACAGGGTGATATCAGAGCACTGCTTTGCGAACAATCTGACAAGGCTTTCTAC | |
| | TCATCCTTCATGGCTTTGATGAGCCTGATGCTCCAGATGAGAAATTCAATTACAGGCAGA | |
| | ACCGACGTGGATTTCTTGATCTCCCCGGTTAAAAATTCTGATGGCATCTTTTACGATAGC | |
| | AGGAACTATGAAGCGCAAGAGAATGCGATTCTGCCAAAAAATGCAGACGCCAACGGTGCC | |
| | TATAACATCGCCAGGAAAGTCCTGTGGGCGATCGGCCAGTTCAAAAAGGCCGAAGACGAA | |
| | AAATTGGACAAGGTCAAAATCGCTATCAGCAACAAAGAGTGGCTGGAGTATGCTCAGACA | |
| | TCCGTAAAGCATAAGCGTCCTGCTGCCACCAAAAAGGCCGGACAGGCTAAGAAAAAGAAG | |
| | TGA | |
| Cas12a | CTTTTTTTCTTCTTCTTCGTTCATACAGTTTTTTTTTGTTTATCAGCTTACATTTTCTTG | 58 |
| guideblock | AACCGTAGCTTTCGTTTTCTTCTTTTTAACTTTCCATTCGGAGTTTTTGTATCTTGTTTC | |
| | ATAGTTTGTCCCAGGATTAGAATGATTAGGCATCGAACCTTCAAGAATTTGATTGAATAA | |
| | AACATCTTCATTCTTAAGATATGAAGATAATCTTCAAAAGGCCCCTGGGAATCTGAAAGA | |
| | AGAGAAGCAGGCCCATTTATATGGGAAAGAACAATAGTATTTCTTATATAGGCCCATTTA | |
| | AGTTGAAAACAATCTTCAAAAGTCCCACATCGCTTAGATAAGAAAACGAAGCTGAGTTTA | |
| | TATACAGCTAGAGTCGAAGTAGTGATTGTAATTTCTACTAAGTGTAGATnnnnnnnnnnn | |
| | nnnnnnnnnnGGCCGGCATGGTCCCAGCCTCCTCGCTGGCGCCGGCTGGGCAACATGCTT | |
| | CGGCATGGCGAATGGGACTTTTTTTTCTAGACCCAGCTTTCTTGTACAAAGTTGGCATTA | |

### Example 3—CRISPR/Cas Gene Editing in Somatic Cannabis Plant Tissue

Efficacy of the above-described vectors for in vivo Cas9 gene editing was validated using a non-embryogenic callus cell line.

Transformation methods. Vectors described herein were introduced into cells using standard transformation methods, as follows: Spherical 1 μm gold particles (BioWorld Inc.) were coated with vector DNA using the 2.5M calcium chloride/0.1 M spermidine conjugation method with a ratio of 2 μg total plasmid DNA per mg of gold. Multiple vectors may have been mixed in equal ratios. For example, in some instances vectors pRI-Cas9-MP18-HygroR, pUC-Cas12a-LB2-HygroR and pUC-Cas12a-LB3-HygroR were mixed for multiplexed editing of CBDAS. In addition to editing vectors, vector pUC-GFP was added to provide a method for assessing transformation rate via transient GFP expression monitored via fluorescent microscopy. Callus to be transformed was grown in bioreactors then placed in a ring in the middle of petri plates containing solid media (1×MS salts and B5 vitamins (PhytoTech Labs), 2% glucose, 0.36% Gelzan, pH5.8) on the day of bombardment. Tissue was biolistically transformed using the PDS-1000/He™ Biolistic Particle Delivery System (Bio-Rad) using 1100 psi rupture disks at 6 cm target distance 1-2 times per plate. Explants were grown overnight at room temperature, transferred to 37 C for 2-3 days, then transferred to selection media.

Selection of clonal callus lines for in vivo editing. Callus was determined to grow at high levels of Kanamycin (300-400 mg/L) and was therefore not useable in these tissues. Hygromycin induced complete callus death in 5-10 mg/L and was chosen for selection.

Callus initiated from vegetative leaf of a CBD-dominant Cannabis genotype was grown in bioreactors under MS and 5 mg/L 6-benzyladenine (BA). The callus was biolistically transformed with pRI and pUC-Cas9 editing vectors (described above) containing various guide RNAs and placed under 10⁻²⁰ mg/L hygromycin selection. Hygromycin resistant clonal callus lines were produced under selection.

Validation of in vivo editing. Lines were screened for editing at CBDAS, CBCAS, R3-MYB1 and R3-MYB2 target genes using Sanger sequencing. Edited cell lines were obtained for each target gene. Some lines presented hemizygous or homozygous edits as insertions/deletions (indels) which were detected when compared to the sequence from the wild-type non-edited callus line.

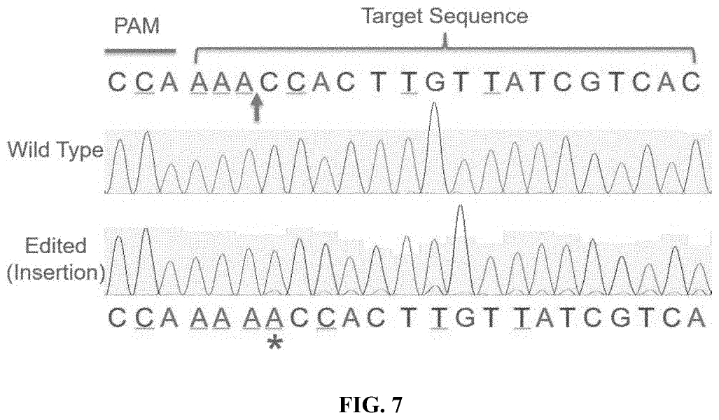

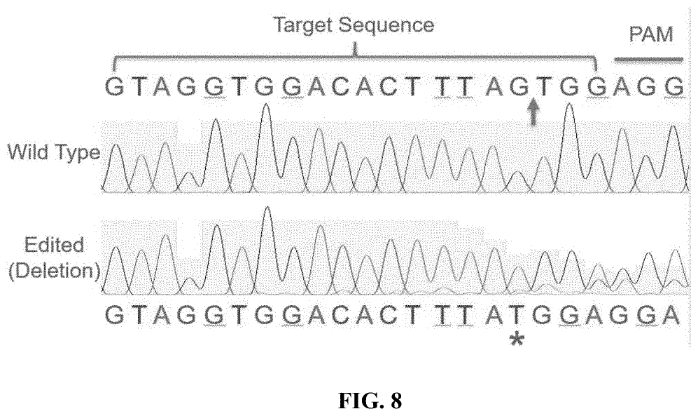

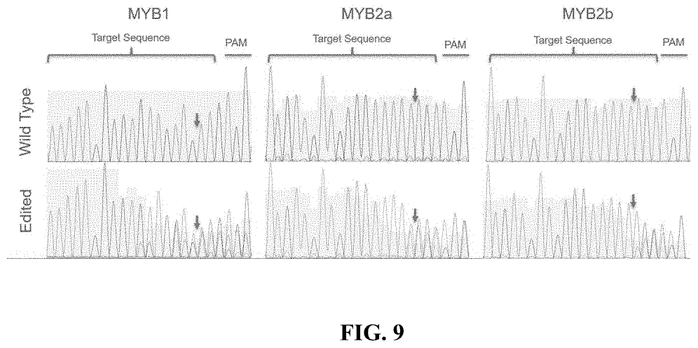

An exemplary result for Cas9 gene editing of the CBDAS gene is shown in FIG. 7. An exemplary result for Cas9 gene editing of the CBCAS gene is shown in FIG. 8. An exemplary result for Cas9 gene editing of the R3-MYB1 and R3-MYB2a/b genes is shown in FIG. 9. The results show insertions or deletions were obtained for each of the target genes.

### Example 4—Establishment of an Immortalized Embryogenic Line for Transgenesis and Gene Editing

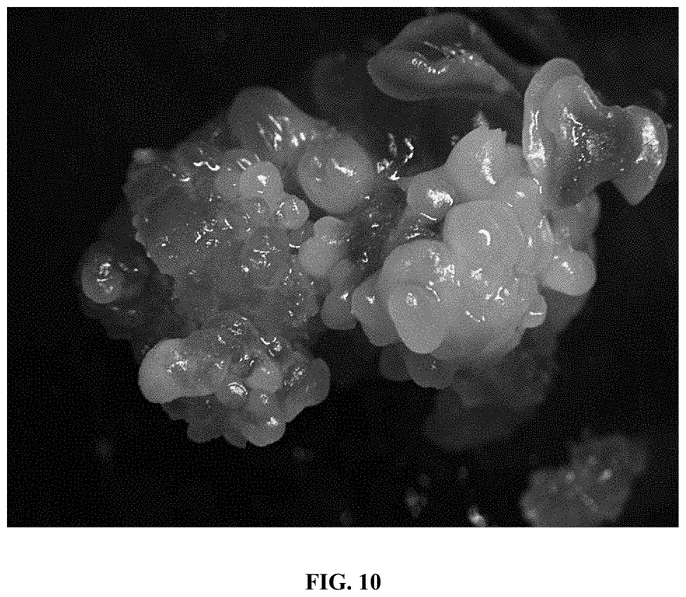

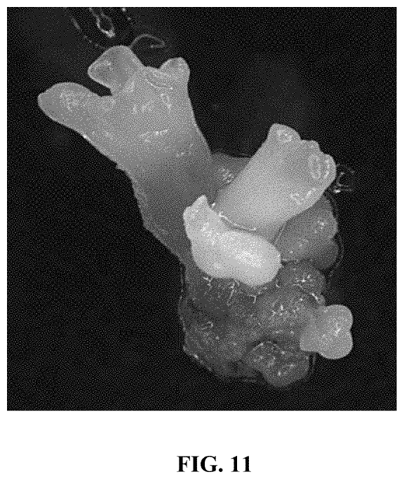

A founding male somatic embryogenic (SE) line from a single Cannabis genotype was established and immortalized by growing in temporary immersion bioreactors and subculturing every 1-2 months as necessary. This line was propagated as compact callus and has the ability to form somatic embryos. The line has been subcultured continually for over two years with no apparent loss in embryogenic capacity. Globular embryos in the male founder SE line are shown in FIG. 10. Heart and torpedo embryos in the male founder SE line are shown in FIG. 11.

### Example 5—Gene Editing in Male Founder SE Line

Transgenesis of immortalized SE lines. To establish transgenic immortalized SE lines, biolistic transformation of the wild type male founder SE line was conducted with Cas9 or Cas12a editing vectors (pRI and pUC) containing hygromycin resistance and either various guide RNA sequences or 35s:eGFP or 35s:AtEGL3 vectors, along with pUC-GFP encoding green fluorescent protein (GFP) for monitoring transformation efficacy via fluorescence microscopy. Transgenic lines with genomic integration of hygromycin resistance were selected on Murashige and Skoog (MS) plates plus 10-20 mg/L hygromycin. Once resistant lines had been established, they were grown indefinitely in RITA® temporary immersion bioreactors (TIBS) under 5-10 mg/L with no apparent loss in embryogenic capacity.

To detect genomic editing in Cas9 or Cas12a vector transformed lines, genomic DNA was extracted and target genes were amplified by PCR and Sanger sequenced. Edits were detected in all target genes tested, as follows: For Cas9 transformed lines, edits were detected in the following target genes: CBDAS, CBCAS, R3-MYB2a/b, R3-MYB3, R3-MYB4, and eIF4e. It is noted that R3-MYB1 was not found in the male founder SE line by gene-specific PCR; and for Cas12a transformed lines, edits were detected in the CBDAS target gene.

SE lines transformed with 35s:eGFP or 35s:AtEGL3 vectors were also analyzed using PCR to confirm genomic integration of the vectors. Genomic integration of 35s::AtEGL3 or 35s::eGFP were confirmed in the transgenic cell lines. Fluorescent microscopy was used to confirm functional expression of GFP in integrated 35s::eGFP lines.

### Example 6—PLANT Regeneration of Male Founder SE Line Edited at CBDAS and Clonal Analysis

Full plant regeneration of a male founder SE line edited at the CBDAS gene, “SE1-11G”, was successfully achieved by somatic embryo maturation and germination.

Embryo induction was carried out by osmotic shocking of bioreactor-grown SE callus on solid MS agar with IM sucrose for 24 hours or occurred spontaneously in bioreactors when grown with 5 mg/L 6-benzyladenine (BA). Induced embryos were then grown on solid MS agar media+20 mg/L hygromycin and continually subcultured every 3-4 weeks until embryos had matured, e.g., had formed a shoot pole with visible cotyledons and a root pole with visible root hairs. Mature embryos were germinated by removing them from attached callus tissue and placing the root pole into media of 2 MS agar.

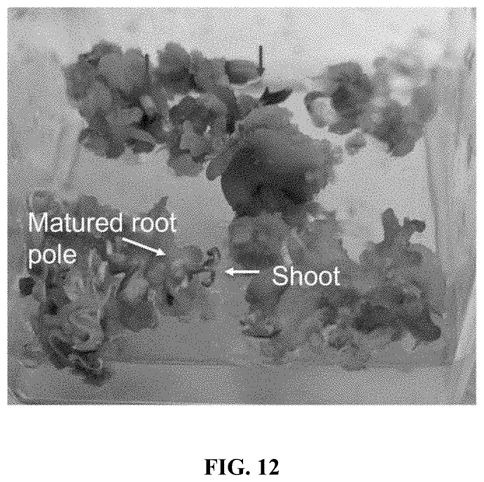

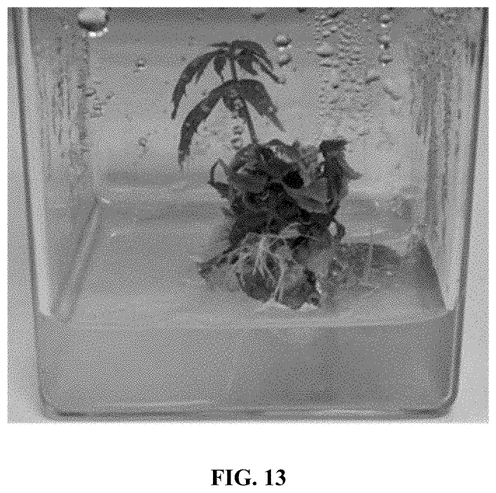

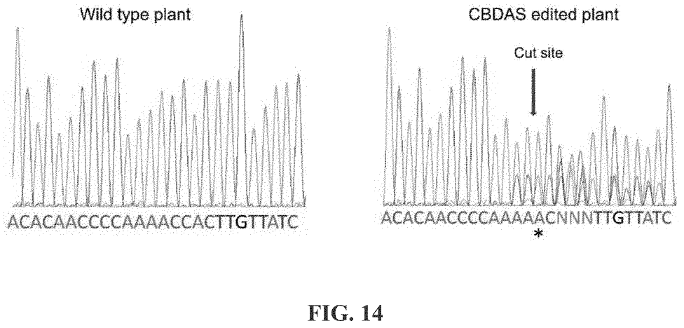

FIG. 12 shows mature embryos formed from CBDAS-edited male founder SE line SE1-11G. FIG. 13 shows a regenerating plant from CBDAS-edited male founder SE line SE1-11G. FIG. 14 shows Sanger Sequencing of the CBDAS gene amplified from genomic DNA of both wild type male SE founder line (left) and regenerating plantlet from CBDAS-edited male founder SE line SE1-11G (pictured in FIG. 13) (right) indicating multiple edited sequences present in the regenerating plantlet, including the insertion of an A nucleotide in the dominant peak trace.

Clonal analysis. Clonal analysis was performed to determine the sequences of edited alleles. First, the wild type CBDAS sequence from the male founder SE line (maleSEfounder_CBDAS) was determined by PCR amplification from callus genomic DNA (gDNA) using Q5 DNA polymerase (New England Bioloabs) and Sanger sequencing, using the following primers: CBDAS forward primer: 5′-CCCTGCTCCAATATATAAAGC-3′ (SEQ ID NO: 59); and CBDAS reverse primer: 5′-ATACACAGTACATCCGGAC-3′ (SEQ ID NO: 60). A single sequence was found without SNPs, having the following sequence:

maleSEfounder_CBDAS:
(SEQ ID NO: 61)
5′-ATGAAGTACTCAACATTCTCCTTTTGGTTTGTTT
GCAAGATAATATTTTTCTTTTTCTCATTCAATATCC
AAACTTCCATTGCTAATCCTCGAGAAAACTTCCTT
AAATGCTTCTCGCAATATATTCCCAATAATGCAAC
AAATCTAAAACTCGTATACACTCAAAACAACCCAT
TGTATATGTCTGTCCTAAATTCGACAATACACAAT
CTTAGATTCAGCTCTGACACAACCCCAAAACCACT
TGTTATCGTCACTCCTTCACATGTCTCTCATATCC
AAGGCACTATTCTATGCTCCAAGAAAGTTGGCTTG
CAGATTCGAACTCGAAGTGGTGGTCATGATTCTGA
GGGCATGTCCTACATATCTCAAGTCCCATTTGTTA
TAGTAGACTTGAGAAACATGCGTTCAATCAAAATA
GATGTTCATAGCCAAACTGCATGGGTTGAAGCCGG
AGCTACCCTTGGAGAAGTTTATTATTGGGTTAATG
AGAAAAATGAGAGTCTTAGTTTGGCTGCTGGGTAT
TGCCCTACTGTTTGCGCAGGTGGACACTTTGGTGG
AGGAGGCTATGGACCATTGATGAGAAGCTATGGCC
TCGCGGCTGATAATATCATTGATGCACACTTAGTC
AACGTTCATGGAAAAGTGCTAGATCGAAAATCTAT
GGGGGAAGATCTCTTTTGGGCTTTACGTGGTGGTG
GAGCAGAAAGCTTCGGAATCATTGTAGCATGGAAA
ATTAGACTGGTTGCTGTCCCAAAGTCTACTATGTT
TAGTGTTAAAAAGATCATGGAGATACATGAGCTTG
TCAAGTTAGTTAACAAATGGCAAAATATTGCTTAC
AAGTATGACAAAGATTTATTACTCATGACTCACTT
CATAACTAGGAACATTACAGATAATCAAGGGAAGA
ATAAGACAGCAATACACACTTACTTCTCTTCAGTT
TTCCTTGGTGGAGTGGATAGTCTAGTCGACTTGAT
GAACAAGAGTTTTCCTGAGTTGGGTATTAAAAAAA
CGGATTGCAGACAATTGAGCTGGATTGATACTATC
ATCTTCTATAGTGGTGTTGTAAATTACGACACTGA
TAATTTTAACAAGGAAATTTTGCTTGATAGATCCG
CTGGGCAGAACGGTGCTTTCAAGATTAAGTTAGAC
TACGTTAAGAAACCAATTCCAGAATCTGTATTTGT
CCAAATTTTGAAAAAATTATATGAAGAAGATATAG
GAGCTGGGATGTATGCGTTGTACCCTTACGGTGGT
ATAATGGATGAGATTTCTGAATCAGCAATTCCATT
CCCTCATCGAGCTGGAATCTTGTATGAGTTATGGT
ACATATGTAGCTGGGAGAAGCAAGAAGATAACGAA
AAGCATCTAAACTGGATTAGAAATATTTATAACTT
CATGACTCCTTATGTGTCCCAAAATCCAAGATTGG
CATATCTCAATTATAGAGACCTTGATATAGGAATA
AATGATCCCAAGAATCCAAATAATTACACACAAGC
ACGTATTTGGGGTGAGAAGTATTTTGGTAAAAATT
TTGACAGGCTAGTAAAAGTGAAAACCCTGGTTGAT
CCCAATAATTTTTTTAGAAACGAACAAAGCATCCC
ACCTCTTCCACGGCATCATCATTAA-3′

The maleSEfounder_CBDAS sequences encodes the following protein sequence:

(SEQ ID NO: 62)
MKYSTFSFWFVCKIIFFFFSFNIQTSIANPRENFL
KCFSQYIPNNATNLKLVYTQNNPLYMSVLNSTIHN
LRFSSDTTPKPLVIVTPSHVSHIQGTILCSKKVGL
QIRTRSGGHDSEGMSYISQVPFVIVDLRNMRSIKI
DVHSQTAWVEAGATLGEVYYWVNEKNESLSLAAGY
CPTVCAGGHFGGGGYGPLMRSYGLAADNIIDAHLV
NVHGKVLDRKSMGEDLFWALRGGGAESFGIIVAWK
IRLVAVPKSTMFSVKKIMEIHELVKLVNKWQNIAY
KYDKDLLLMTHFITRNITDNQGKNKTAIHTYFSSV
FLGGVDSLVDLMNKSFPELGIKKTDCRQLSWIDTH
IFYSGVVNYDTDNFNKEILLDRSAGQNGAFKIKLD
YVKKPIPESVFVQILKKLYEEDIGAGMYALYPYGG
IMDEISESAIPFPHRAGILYELWYICSWEKQEDNE
KHLNWIRNIYNFMTPYVSQNPRLAYLNYRDLDIGI
NDPKNPNNYTQARIWGEKYFGKNFDRLVKVKTLVD
PNNFFRNEQSIPPLPRHHH

The maleSEfounder_CBDAS sequence contains a novel SNP 1201A in the CBDAS coding sequence that was not found in any deposited nucleotide sequence on NCBI by BLAST search. The 1201A SNP produces a novel amino acid change (401K) in the CBDAS protein sequence which was also not found in any deposited amino acid sequence on NCBI by BLAST search.

CBDAS sequences from edited plantlet “11G” (which is the plantlet from CBDAS-edited male founder SE line SE1-11G, shown in FIGS. 13 and 14) were PCR amplified using Q5 polymerase and the following primers: CBDASfMCS1 forward primer: 5′-AGCAAGTTCTTCACTGTTGATACATCCCTGCTCCAATATATAAAGC-3′ (SEQ ID NO: 63); and CBDASrMCS1 reverse primer: 5′-GAGTTGTTGATTCAGAATTGTCGACATACACAGTACATCCGGAC-3′ (SEQ ID NO: 64).

Amplicons were cloned into MCS1 of vector pUC and transformed into competent E. coli. 11 individual colonies were selected, DNA isolated therefrom and Sanger sequenced. Three CBDAS sequences (11G1-3) were found, each containing a unique edit at the cut site for MP18, as follows:

11G1_CBDAS:
(SEQ ID NO: 65)
5′-
ATGAAGTACTCAACATTCTCCTTTTGGTTTGTTTG
CAAGATAATATTTTTCTTTTTCTCATTCAATATCC
AAACTTCCATTGCTAATCCTCGAGAAAACTTCCTT
AAATGCTTCTCGCAATATATTCCCAATAATGCAAC
AAATCTAAAACTCGTATACACTCAAAACAACCCAT
TGTATATGTCTGTCCTAAATTCGACAATACACAAT
CTTAGATTCAGCTCTGACACAACCCCAAAAaCCA
CTTGTTATCGTCACTCCTTCACATGTCTCTCA
TATCCAAGGCACTATTCTATGCTCCAAGAAAGTTG
GCTTGCAGATTCGAACTCGAAGTGGTGGTCATGAT
TCTGAGGGCATGTCCTACATATCTCAAGTCCCATT
TGTTATAGTAGACTTGAGAAACATGCGTTCAATCA
AAATAGATGTTCATAGCCAAACTGCATGGGTTGAA
GCCGGAGCTACCCTTGGAGAAGTTTATTATTGGGT
TAATGAGAAAAATGAGAGTCTTAGTTTGGCTGCTG
GGTATTGCCCTACTGTTTGCGCAGGTGGACACTTT
GGTGGAGGAGGCTATGGACCATTGATGAGAAGCTA
TGGCCTCGCGGCTGATAATATCATTGATGCACACT
TAGTCAACGTTCATGGAAAAGTGCTAGATCGAAAA
TCTATGGGGGAAGATCTCTTTTGGGCTTTACGTGG
TGGTGGAGCAGAAAGCTTCGGAATCATTGTAGCAT
GGAAAATTAGACTGGTTGCTGTCCCAAAGTCTACT
ATGTTTAGTGTTAAAAAGATCATGGAGATACATGA
GCTTGTCAAGTTAGTTAACAAATGGCAAAATATTG
CTTACAAGTATGACAAAGATTTATTACTCATGACT
CACTTCATAACTAGGAACATTACAGATAATCAAGG
GAAGAATAAGACAGCAATACACACTTACTTCTCTT
CAGTTTTCCTTGGTGGAGTGGATAGTCTAGTCGAC
TTGATGAACAAGAGTTTTCCTGAGTTGGGTATTAA
AAAAACGGATTGCAGACAATTGAGCTGGATTGATA
CTATCATCTTCTATAGTGGTGTTGTAAATTACGAC
ACTGATAATTTTAACAAGGAAATTTTGCTTGATAG
ATCCGCTGGGCAGAACGGTGCTTTCAAGATTAAGT
TAGACTACGTTAAGAAACCAATTCCAGAATCTGTA
TTTGTCCAAATTTTGAAAAAATTATATGAAGAAGA
TATAGGAGCTGGGATGTATGCGTTGTACCCTTACG
GTGGTATAATGGATGAGATTTCTGAATCAGCAATT
CCATTCCCTCATCGAGCTGGAATCTTGTATGAGTT
ATGGTACATATGTAGCTGGGAGAAGCAAGAAGATA
ACGAAAAGCATCTAAACTGGATTAGAAATATTTAT
AACTTCATGACTCCTTATGTGTCCCAAAATCCAAG
ATTGGCATATCTCAATTATAGAGACCTTGATATAG
GAATAAATGATCCCAAGAATCCAAATAATTACACA
CAAGCACGTATTTGGGGTGAGAAGTATTTTGGTAA
AAATTTTGACAGGCTAGTAAAAGTGAAAACCCTGG
TTGATCCCAATAATTTTTTTAGAAACGAACAAAGC
ATCCCACCTCTTCCACGGCATCATCATTAA-3′
11G2_CBDAS:
(SEQ ID NO: 66)
5′-ATGAAGTACTCAACATTCTCCTTTTGGTTTGTT
TGCAAGATAATATTTTTCTTTTTCTCATTCAATATC
CAAACTTCCATTGCTAATCCTCGAGAAAACTTCCTT
AAATGCTTCTCGCAATATATTCCCAATAATGCAAC
AAATCTAAAACTCGTATACACTCAAAACAACCCAT
TGTATATGTCTGTCCTAAATTCGACAATACACAAT
CTTAGATTCAGCTCTGACACAACCCCAACCACTTG
TTATCGTCACTCCTTCACATGTCTCTCATATCCAA
GGCACTATTCTATGCTCCAAGAAAGTTGGCTTGCA
GATTCGAACTCGAAGTGGTGGTCATGATTCTGAGG
GCATGTCCTACATATCTCAAGTCCCATTTGTTATA
GTAGACTTGAGAAACATGCGTTCAATCAAAATAGA
TGTTCATAGCCAAACTGCATGGGTTGAAGCCGGAG
CTACCCTTGGAGAAGTTTATTATTGGGTTAATGAG
AAAAATGAGAGTCTTAGTTTGGCTGCTGGGTATTG
CCCTACTGTTTGCGCAGGTGGACACTTTGGTGGAG
GAGGCTATGGACCATTGATGAGAAGCTATGGCCTC
GCGGCTGATAATATCATTGATGCACACTTAGTCAA
CGTTCATGGAAAAGTGCTAGATCGAAAATCTATGG
GGGAAGATCTCTTTTGGGCTTTACGTGGTGGTGGA
GCAGAAAGCTTCGGAATCATTGTAGCATGGAAAAT
TAGACTGGTTGCTGTCCCAAAGTCTACTATGTTTA
GTGTTAAAAAGATCATGGAGATACATGAGCTTGTC
AAGTTAGTTAACAAATGGCAAAATATTGCTTACAA
GTATGACAAAGATTTATTACTCATGACTCACTTCA
TAACTAGGAACATTACAGATAATCAAGGGAAGAAT
AAGACAGCAATACACACTTACTTCTCTTCAGTTTT
CCTTGGTGGAGTGGATAGTCTAGTCGACTTGATGA
ACAAGAGTTTTCCTGAGTTGGGTATTAAAAAAACG
GATTGCAGACAATTGAGCTGGATTGATACTATCAT
CTTCTATAGTGGTGTTGTAAATTACGACACTGATA
ATTTTAACAAGGAAATTTTGCTTGATAGATCCGCT
GGGCAGAACGGTGCTTTCAAGATTAAGTTAGACTA
CGTTAAGAAACCAATTCCAGAATCTGTATTTGTCC
AAATTTTGAAAAAATTATATGAAGAAGATATAGGA
GCTGGGATGTATGCGTTGTACCCTTACGGTGGTAT
AATGGATGAGATTTCTGAATCAGCAATTCCATTCC
CTCATCGAGCTGGAATCTTGTATGAGTTATGGTAC
ATATGTAGCTGGGAGAAGCAAGAAGATAACGAAAA
GCATCTAAACTGGATTAGAAATATTTATAACTTCA
TGACTCCTTATGTGTCCCAAAATCCAAGATTGGCA
TATCTCAATTATAGAGACCTTGATATAGGAATAAA
TGATCCCAAGAATCCAAATAATTACACACAAGCAC
GTATTTGGGGTGAGAAGTATTTTGGTAAAAATTTT
GACAGGCTAGTAAAAGTGAAAACCCTGGTTGATCC
CAATAATTTTTTTAGAAACGAACAAAGCATCCCAC
CTCTTCCACGGCATCATCATTAA-3′
11G3 CBDAS:
(SEQ ID NO: 67)
5′-ATGAAGTACTCAACATTCTCCTTTTGGTTTGTT
TGCAAGATAATATTTTTCTTTTTCTCATTCAATATC
CAAACTTCCATTGCTAATCCTCGAGAAAACTTCCTT
AAATGCTTCTCGCAATATATTCCCAATAATGCAAC
AAATCTAAAACTCGTATACACTCAAAACAACCCAT
TGTATATGTCTGTCCTAAATTCGACAATACACAAT
CTTAGATTCAGCTCTGACACAACCCCAAAATTGTT
ATCGTCACTCCTTCACATGTCTCTCATATCCAAGG
CACTATTCTATGCTCCAAGAAAGTTGGCTTGCAGA
TTCGAACTCGAAGTGGTGGTCATGATTCTGAGGGC
ATGTCCTACATATCTCAAGTCCCATTTGTTATAGT
AGACTTGAGAAACATGCGTTCAATCAAAATAGATG
TTCATAGCCAAACTGCATGGGTTGAAGCCGGAGCT
ACCCTTGGAGAAGTTTATTATTGGGTTAATGAGAA
AAATGAGAGTCTTAGTTTGGCTGCTGGGTATTGCC
CTACTGTTTGCGCAGGTGGACACTTTGGTGGAGGA
GGCTATGGACCATTGATGAGAAGCTATGGCCTCGC
GGCTGATAATATCATTGATGCACACTTAGTCAACG
TTCATGGAAAAGTGCTAGATCGAAAATCTATGGGG
GAAGATCTCTTTTGGGCTTTACGTGGTGGTGGAGC
AGAAAGCTTCGGAATCATTGTAGCATGGAAAATTA
GACTGGTTGCTGTCCCAAAGTCTACTATGTTTAGT
GTTAAAAAGATCATGGAGATACATGAGCTTGTCAA
GTTAGTTAACAAATGGCAAAATATTGCTTACAAGT
ATGACAAAGATTTATTACTCATGACTCACTTCATA
ACTAGGAACATTACAGATAATCAAGGGAAGAATAA
GACAGCAATACACACTTACTTCTCTTCAGTTTTCC
TTGGTGGAGTGGATAGTCTAGTCGACTTGATGAAC
AAGAGTTTTCCTGAGTTGGGTATTAAAAAAACGGA
TTGCAGACAATTGAGCTGGATTGATACTATCATCT
TCTATAGTGGTGTTGTAAATTACGACACTGATAAT
TTTAACAAGGAAATTTTGCTTGATAGATCCGCTGG
GCAGAACGGTGCTTTCAAGATTAAGTTAGACTACG
TTAAGAAACCAATTCCAGAATCTGTATTTGTCCAA
ATTTTGAAAAAATTATATGAAGAAGATATAGGAGC
TGGGATGTATGCGTTGTACCCTTACGGTGGTATAA
TGGATGAGATTTCTGAATCAGCAATTCCATTCCCT
CATCGAGCTGGAATCTTGTATGAGTTATGGTACAT
ATGTAGCTGGGAGAAGCAAGAAGATAACGAAAAGC
ATCTAAACTGGATTAGAAATATTTATAACTTCATG
ACTCCTTATGTGTCCCAAAATCCAAGATTGGCATA
TCTCAATTATAGAGACCTTGATATAGGAATAAATG
ATCCCAAGAATCCAAATAATTACACACAAGCACGT
ATTTGGGGTGAGAAGTATTTTGGTAAAAATTTTGA
CAGGCTAGTAAAAGTGAAAACCCTGGTTGATCCCA
ATAATTTTTTTAGAAACGAACAAAGCATCCCACCT
CTTCCACGGCATCATCATTAA-3′

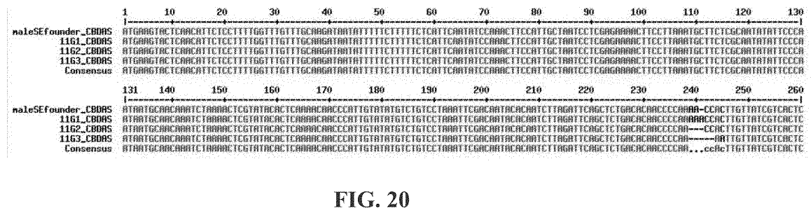

An alignment of the wild type CBDAS and the three edited allele nucleotide sequences (11G1_CBDAS, 11G2_CBDAS, 11G3_CBDAS) is presented in FIG. 20. 11G1 contained an A insertion at position 240. 11G2 contained a 2-nucleotide deletion beginning at position 238. 11G3 contained a 4-nucleotide deletion beginning at position 241. Overlay of these three edited alleles accounts for the observed nucleotide pattern presented in FIG. 14 for the edited plantlet. Further, since more than two alleles were found and cannabis is a diploid genotype, these results suggest that CBDAS is duplicated and homozygous in the male founder SE lines with a total of 4 CBDAS gene copies containing identical coding sequences. The weight of peaks in the chromatogram in FIG. 14, combined with the prevalence of 11G1 being present in approximately half (7/11) of the cloned sequences, further suggests that two copies of 11G1 were present in addition to a single copy of 11G2 and 11G3 in the regenerated plantlet. Regardless of copy number, no wild type allele was found in the edited plantlet using clonal sequence analysis, suggesting complete editing of all CBDAS alleles present that were amplified by the primer sets used here.

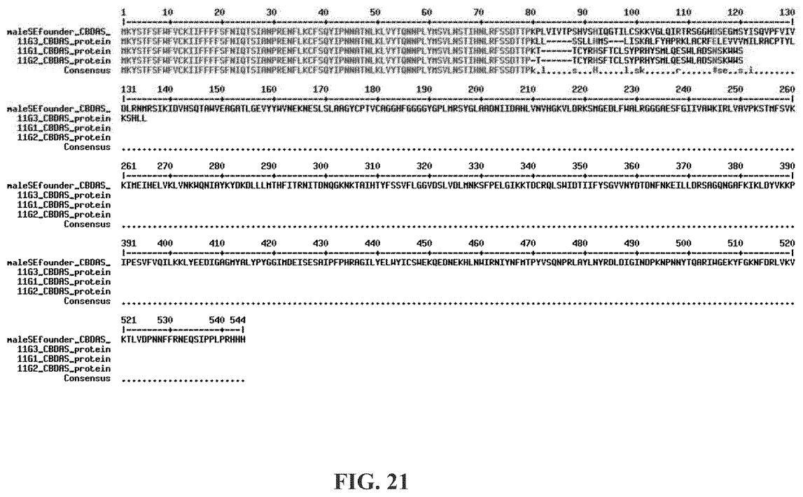

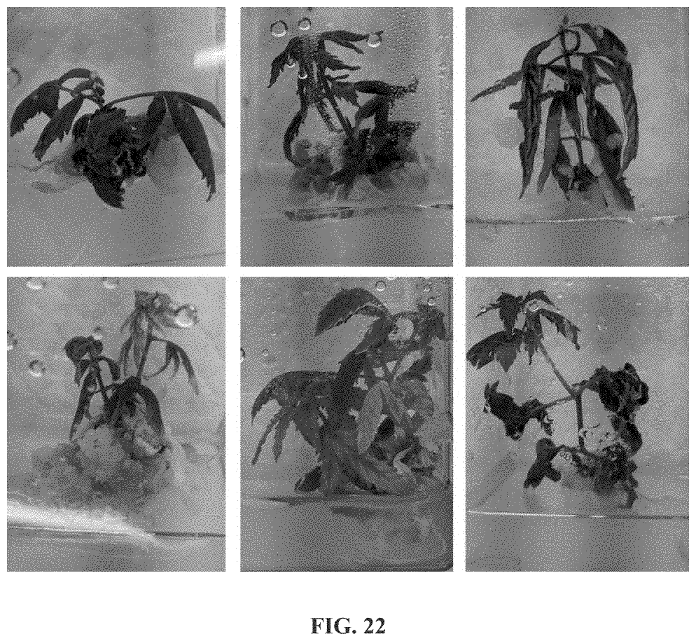

Translation of the coding sequences for CBDAS 11G1, 11G2, and 11G3 showed that all three alleles had frameshifts beginning at amino acid positions 80 or 81 leading to early stop codons, and presumably inactive protein products. An alignment of the wild type CBDAS amino acid sequence from the male founder SE line with the amino acid sequences for the three edited 11G alleles (11G1_CBDAS, 11G2_CBDAS, 11G3_CBDAS) is presented in FIG. 21. The sequences are as follows:

11G1_CBDAS_protein:
(SEQ ID NO: 68)
MKYSTFSFWFVCKIIFFFFSFNIQTSIANPRENFL
KCFSQYIPNNATNLKLVYTQNNPLYMSVLNSTIHN
LRFSSDTTPKTTCYRHSFTCLSYPRHYSMLQESWL
ADSNSKWWS.
11G2_CBDAS_protein:
(SEQ ID NO: 69)
MKYSTFSFWFVCKIIFFFFSFNIQTSIANPRENFL
KCFSQYIPNNATNLKLVYTQNNPLYMSVLNSTIHN
LRFSSDTTPTTCYRHSFTCLSYPRHYSMLQESWLA
DSNSKWWS.
11G3_CBDAS_protein:
(SEQ ID NO: 70)
MKYSTFSFWFVCKIIFFFFSFNIQTSIANPRENFL
KCFSQYIPNNATNLKLVYTQNNPLYMSVLNSTIHN
LRFSSDTTPKLLSSLLHMSLISKALFYAPRKLACR
FELEVVVMILRACPTYLKSHLL.

### Example 7—Establishment of Female Embryogenic Lines from Male SE Founder Progeny

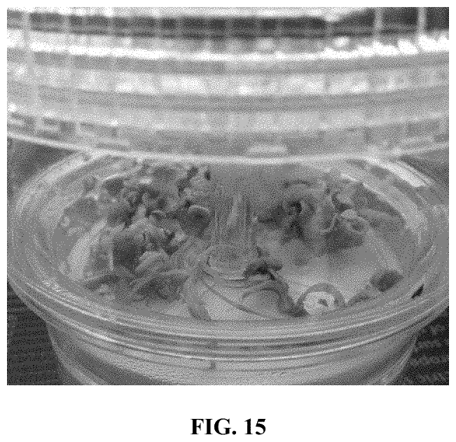

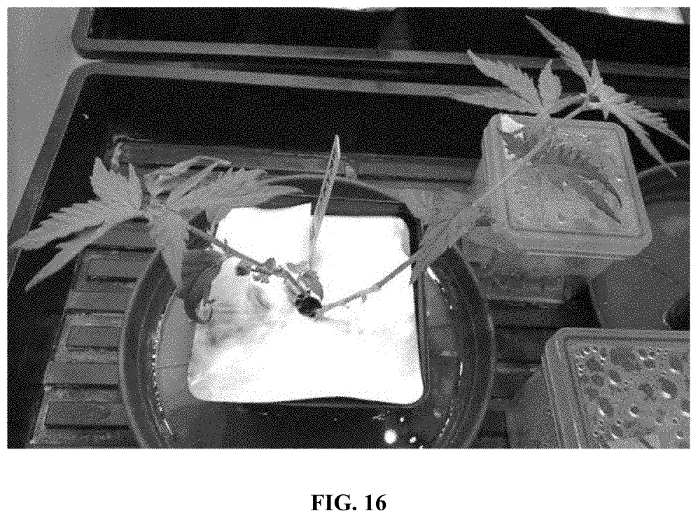

Shoots of the male founder SE lines developed spontaneously in bioreactors when grown under 5 mg/L BA. Shoots were transferred to MS agar boxes with indole-3-butyric acid (IBA) to induce rooting. Rooted shoots were hardened and full plants were established. The full plants produced anthers and pollen, which were applied to a female flowering CBD-dominant hemp strain. FIG. 15 shows shoots of the wild type male founder SE line in a bioreactor. FIG. 16 shows a hardened male founder SE line plant.

F1 seeds were produced and 4 seeds were germinated and established and confirmed as female. Plants were flowered and selfed using silver thiosulphate sprays. Seeds were collected from the S1 of F1 plants and cotyledons of seeds were induced for SEs. Three new female embryogenic lines (named SE2a, SE4c, and SE10b) were induced and grown in bioreactors under 5 mg/L BA. When moved to hormone-free media these SEs developed shoots that were further clonally propagated.

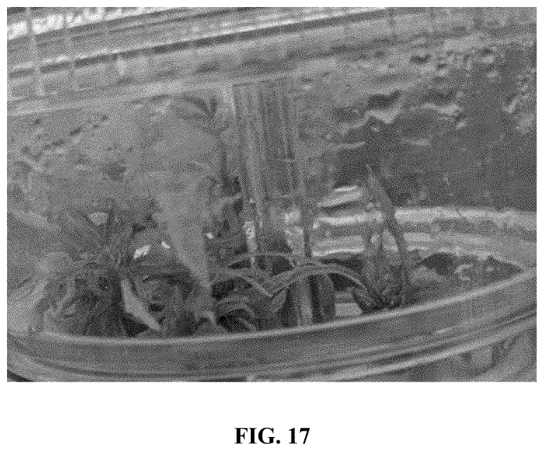

FIG. 17 shows shoots of a female derived SE line (line SE2a) grown in a bioreactor.

### Example 8—Transformation and Selection of Derived SE Lines

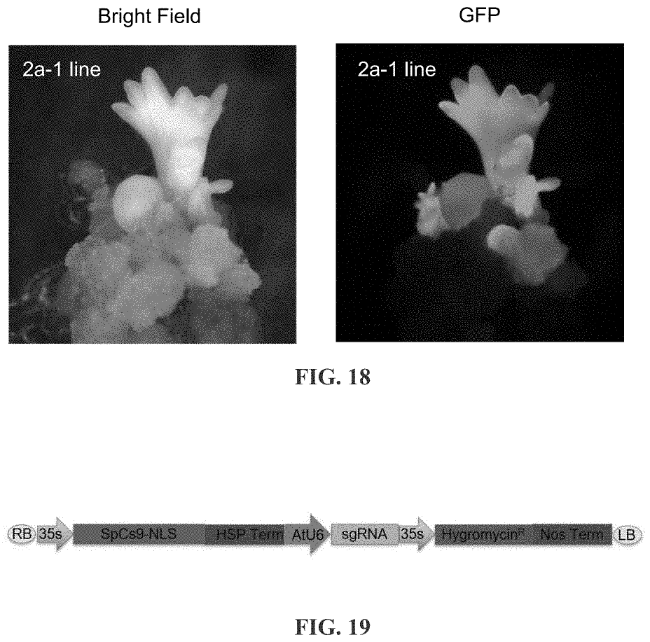

Female derived SE line SE2a was biolistically transformed with a GFP-containing vector pUC-GFP-HygroR and selected under 20 mg/L hygromycin. A GFP-positive line SE2a-1 was identified under fluorescent microscopy and monitored during development. GFP-positive SEs developed when grown on MS plates under 5 mg/L BA. FIG. 18 shows bright field microscopy (left) and fluorescent microscopy (right) of GFP-positive SEs from line SE2a-1.

### Example 9—Flowering and Pollen Production in Plants of Male Founder SE Line Edited at CBDAS

Several SE1-11G somatic embryos were regenerated into rooted plantlets in tissue culture as described above. Further examples of regenerated plantlets are shown in FIG. 22.

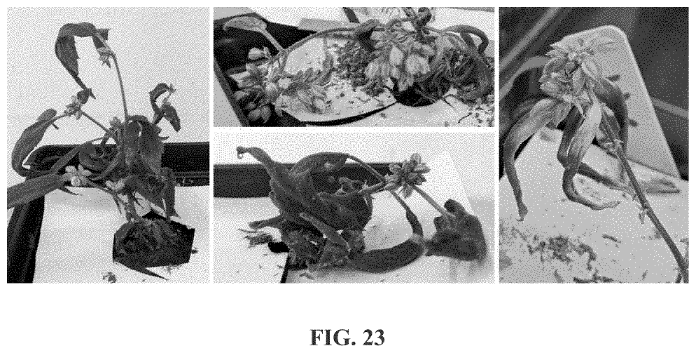

Once plantlets had established sufficient roots in tissue culture, they were transferred to rockwool (Grodan) and hardened. All hardened plantlets spontaneously formed anthers under 18:6 light:dark conditions. Examples of hardened SE-11G plantlets with anthers are shown in FIG. 23.

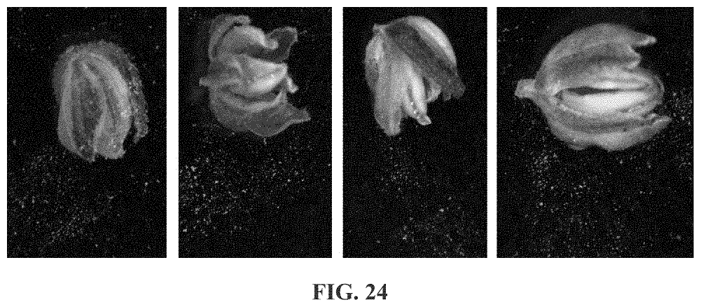

Anthers of hardened flowering SE1-11G plants produced and released pollen. Mature anthers were removed from flowering plants and gently agitated and placed on petri dishes, then imaged with a stereomicroscope against a black background to visualize pollen. Examples of anthers and released pollen from SE1-11G plants are shown in FIG. 24. Pollen grains can be seen as small yellow structures around the anthers.

### Example 10—Establishment of Female Derived SE Lines

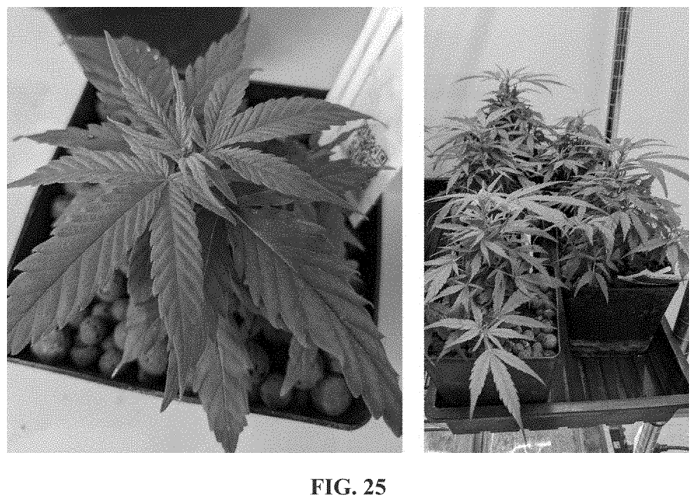

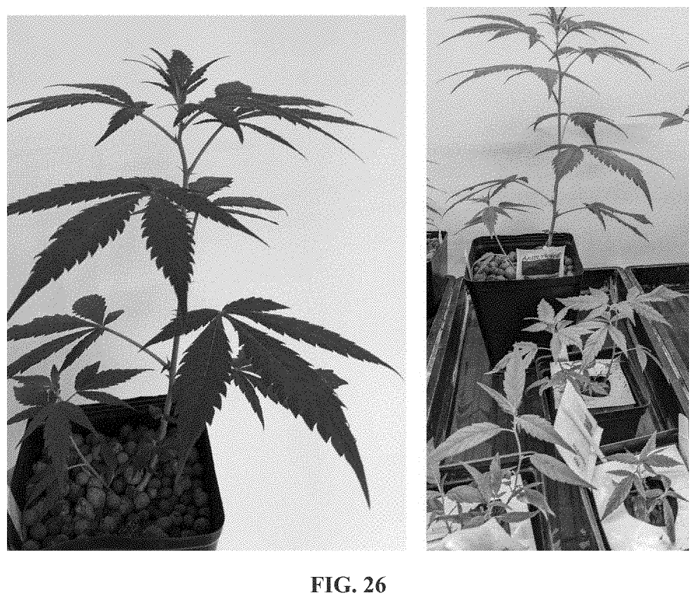

Shoots of female derived SE lines SE2a and SE4c that were clonally propagated in bioreactors were transferred to MS agar boxes with indole-3-butyric acid (IBA) to induce rooting. Rooted plants were hardened and full plants were established. FIG. 25 shows established vegetative plants of female derived line SE2a. FIG. 26 shows established vegetative plants of female derived line SE4c. Vegetative SE2a and SE4c plants were further clonally propagated.

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

### REFERENCES

Ausubel, et al., Short Protocols in Molecular Biology, 3rd ed., Wiley & Sons, 1995.
Belhaj, K., Chaparro-Garcia, A., Kamoun, S., Patron, N. J., and Nekrasov, V. (2015). Editing plant genomes with CRISPR/Cas9. Curr. Opin. Biotechnol. 32:76-84.
Bortesi, L., and Fischer, R. (2015). The CRISPR/Cas9 system for plant genome editing and beyond. Biotechnol. Adv. 33:41-52.
Burgess, D. J. (2013). Technology: a CRISPR genome-editing tool. Nat. Rev. Genet. 14:80. doi: 10.1038/nrg3409.
Cong, L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science 339:819-823.
Di Marzo, V., & Piscitelli, F. (2015). The Endocannabinoid System and its Modulation by Phytocannabinoids. In Neurotherapeutics. https://doi.org/10.1007/s13311-015-0374-6.
Doudna, J. A. and Charpentier, E. (2014) Genome editing. The new frontier of genome engineering with CRISPR-Cas9. Science 346(6213):1258096. doi: 10.1126/science.1258096.
ElSohly, Mahmoud A., Mohamed M. Radwan, Waseem Gul, Suman Chandra, and Ahmed Galal. “Phytochemistry of Cannabis sativa L.” Phytocannabinoids (2017): 1-36.
Fagerlund, R. D., Staals, R. H., and Fineran, P. C. (2015). The Cpf1 CRISPR-Cas protein expands genome-editing tools. Genome Biol. 16:251. doi: 10.1186/s13059-015-0824-9.
Farnsworth, Norman R. “Pharmacognosy and Chemistry of “Cannabis Sativa”.” Journal of the American Pharmaceutical Association 9, no. 8 (1969): 410.
Fonfara, I., Richter, H., Bratovic, M., Le Rhun, A., and Charpentier, E. (2016). The CRISPR-associated DNA-cleaving enzyme Cpf1 also processes precursor CRISPR RNA. Nature 532, 517-521. doi: 10.1038/nature17945.
Fournier, G., Richez-Dumanois, C., Duvezin, J., Mathieu, J. P., & Paris, M. (1987). Identification of a new chemotype in Cannabis sativa: Cannabigerol-dominant plants, biogenetic and agronomic prospects. PlantaMedica. https://doi.org/10.1055/s-2006-962705
Jiang, F., Zhou, K., Ma, L., Gressel, S., and Doudna, J. A. (2015). STRUCTURAL BIOLOGY. A Cas9-guide RNA complex preorganized for target DNA recognition. Science 348:1477-1481.
Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337, 816-21. doi: 10.1126/science.1225829. Epub 2012 Jun. 28.
Kaya, H., Mikami, M., Endo, A., Endo, M., and Toki, S. (2016). Highly specific targeted mutagenesis in plants using Staphylococcus aureus Cas9 Sci. Rep, 6:26871 doi: 10.1038/srep26871.
Kumar, V., and Jain, M. (2015). The CRISPR-Cas system for plant genome editing: advances and opportunities. J. Exp. Bot. 66:47-57.
Kumar, S., AlAbed, D., Worden, A., Novak, S., Wu, H., Ausmus, C., Beck, M., Robinson, H., Minicks, T., Hemingway, D., et al. (2015). A modular gene targeting system for sequential transgene stacking in plants. J. Biotechnol. 207:12-20.
Kuromori, T., Wada, T., Kamiya, A., Yuguchi, M., Yokouchi, T., Imura, Y., Takabe, H., Sakurai, T., Akiyama, K., Hirayama, T., et al. (2006). A trial of phenome analysis using 4000 Ds-insertional mutants in gene coding regions of Arabidopsis. Plant J. 47:640-651.
Lloyd, A., Plaisier, C. L., Carroll, D., and Drews, G. N. (2005). Targeted mutagenesis using zinc-finger nucleases in Arabidopsis. Proc. Natl. Acad. Sci. USA 102:2232-2237.
Ma, X., Zhu, Q., Chen, Y, and Liu, Y-G. (2016). CRISPR/Cas9 Platforms for Genome Editing in Plants: Developments and Applications. Molecular Plant 9, 961-974. doi: 10.1016/j.molp.2016.04.009.
McPartland, J. M., & Guy, G. W. (2017). Models of Cannabis Taxonomy, Cultural Bias, and Conflicts between Scientific and Vernacular Names. Botanical Review. https://doi.org/10.1007/s12229-017-9187-0.
McPartland, J. M., & Small, E. (2020). A classification of endangered high-THC Cannabis (Cannabis sativa subsp. indica) domesticates and their wild relatives. PhytoKeys. https://doi.org/10.3897/PHYTOKEYS.144.46700.
Nakade, S., Yamamoto, T., and Sakuma, T. (2017). Cas9, Cpf1 and C2c1/2/3-What's next? Bioengineered 8, 265-273. doi: 10.1080/21655979.2017.1282018.
Osakabe, Y., and Osakabe, K. (2015). Genome editing with engineered nucleases in plants. Plant Cell Physiol. 56:389-400.
Puchta, H., and Fauser, F. (2014). Synthetic nucleases for genome engineering in plants: prospects for a bright future. Plant J. 78:727-741.
Schultes, R. E., Schultes, R. E., Klein, W. M., Plowman, T., & Lockwood, T. E. (1974). Cannabis: an example of taxonomic neglect. Botanical Museum Leaflets, Harvard University.
Shan, Q., and Gao, C. (2015). Research progress of genome editing and derivative technologies in plants. Hereditas (Beijing) 37:953-973.
Small, Ernest. “Interfertility and chromosomal uniformity in Cannabis.” Canadian Journal of Botany 50, no. 9 (1972): 1947-1949.
Small, E., & Beckstead, H. D. (1973). Cannabinoid phenotypes in Cannabis sativa. Nature. https://doi.org/10.1038/245147a0.
Small, Ernest, and Arthur Cronquist. “A practical and natural taxonomy for Cannabis.” Taxon (1976): 405-435.
Sternberg, S. H., Redding, S., Jinek, M., Greene, E. C., and Doudna, J. A. (2014). DNA interrogation by the CRISPR RNA-guided endonuclease Cas9. Nature 507:62-67.
Symington, L. S., and Gautier, J. (2011). Double-strand break end resection and repair pathway choice. Annu. Rev. Genet. 45:247-271.
Turnbull, J. W., Johnston, E. L., and Clark, G. F. (2021). Evaluating the social and ecological effectiveness of partially protected marine areas. Conserv. Biol. 35, 921-932. doi: 10.1111/cobi.13677.
Voytas, D. F. (2013). Plant genome engineering with sequence-specific nucleases. Annu. Rev. Plant Biol. 64:327-350.
Wu, C., Li, X., Yuan, W., Chen, G., Kilian, A., Li, J., Xu, C., Li, X., Zhou, D. X., Wang, S., et al. (2003). Development of enhancer trap lines for functional analysis of the rice genome. Plant J. 35:418-427.
Xie, K., and Yang, Y. (2013). RNA-guided genome editing in plants using a CRISPR-Cas system. Mol. Plant 6:1975-1983.
Wu, Y., Yuan, Q, Zhu, Y., Gao, X., and Yin, Z. (2020) Improving FnCas12a genome editing by exonuclease fusion. CRISPR J. 3, 503-511. doi: 10.1089/crispr.2020.0073.

What is claimed is:

1. A method for genetically editing a target gene in the genome of cells from a Cannabis plant, the method comprising:
(a) introducing one or more exogenous nucleic acid having gene-editing activity into the cells;
(b) optionally, cultivating the cells under conditions allowing gene-editing of the target gene in the genome of said cells; and
(c) optionally, selecting cells which have been gene-edited by the gene-editing activity of the one or more exogenous nucleic acid molecule.

2. The method of claim 1, wherein the one or more exogenous nucleic acid molecule is a DNA.

3. The method of claim 2, wherein the DNA is an expression vector.

4. The method of claim 1, wherein the one or more exogenous nucleic acid molecule is an RNA.

5. The method of claim 1, wherein the one or more exogenous nucleic acid molecule encodes a CRISPR endonuclease.

6. The method of claim 5, wherein the CRISPR endonuclease is a Cas enzyme, optionally selected from Cas9, Cas12, Cas12a, Cas13a, Cpf1, Csm1, CasX, and CasY.

7. The method of claim 6, wherein the Cas enzyme is Cas9, optionally selected from SpCas9, SaCas9, SauriCas9, StCas9, NmCas9, and FnCas9.

8. The method of claim 7, wherein the Cas9 is SpCas9-NLS.

9. The method of claim 6, wherein the Cas enzyme is Cas12, optionally Cas12a, optionally selected from AsCas12a, FnCas12a, and LbCas12a.

10. The method of claim 9, wherein the Cas12 is LbCas12a-NLS.

11. The method of claim 1, wherein the one or more exogenous nucleic acid molecule encodes a guide RNA, optionally selected from a crRNA, a tracrRNA, an sgRNA, and combinations thereof.

12. The method of claim 4, wherein the one or more exogenous nucleic acid molecule is a guide RNA, optionally selected from a crRNA, a tracrRNA, an sgRNA, and combinations thereof.

13. The method of claim 11, wherein the guide RNA comprises or consists of the sequence set forth in any one of SEQ ID NOs: 1-42, or has at least, greater than or about 75%, 80%, 85%, 90%, 95%, 96%, 97%, 98% or 99% sequence identity thereto.

14. The method of claim 13, wherein the guide RNA has the sequence set forth in any one of SEQ ID NOs: 1-42.

15. The method of claim 11, wherein the guide RNA binds the target gene in the genome of the cells, wherein the target gene is CBDAS, CBCAS, R3-MYB1, R3-MYB2, R3-MYB3, R3-MYB4, or eIF4e.

16. The method of claim 1, wherein the target gene is CBDAS, CBCAS, R3-MYB1, R3-MYB2, R3-MYB3, R3-MYB4, eIF4e, or a combination thereof.

17. The method of claim 1, wherein the one or more exogenous nucleic acid molecule comprises a first exogenous nucleic acid molecule and a second exogenous nucleic acid molecule, the first exogenous nucleic acid molecule encoding the CRISPR endonuclease as defined in claim 6, and the second exogenous nucleic acid molecule encoding the guide RNA as defined in claim 11.

18. The method of claim 1, wherein said step of introducing one or more exogenous nucleic acid having gene-editing activity into the cells comprises transformation, optionally transformation via a DNA virus, via an RNA virus, via protoplasts, via T-DNA delivery or via particle bombardment.

19. The method of claim 18, wherein said transformation comprises transformation with an Agrobacterium carrying said one or more exogenous nucleic acid molecule.

20. The method of claim 18, wherein said transformation comprises transformation via biolistic transformation.


SEQUENCE LISTING (70 sequences)

SEQ ID NO: 1 - DNA, 20 bp, synthetic construct
  source 1..20: mol_type = other DNA
gaaggagtgacaataacgag

SEQ ID NO: 2 - DNA, 20 bp, Synthetic construct
  source 1..20: mol_type = other DNA
gtaggtggacactttagtgg

SEQ ID NO: 3 - DNA, 20 bp, synthetic construct
  source 1..20: mol_type = other DNA
gaaggagtgacgataacaag

SEQ ID NO: 4 - DNA, 20 bp, synthetic construct
  source 1..20: mol_type = other DNA
gcaggtggacactttggtgg

SEQ ID NO: 5 - DNA, 20 bp, synthetic construct
  source 1..20: mol_type = other DNA
gttttctcgaggattagcaa

SEQ ID NO: 6 - DNA, 20 bp, synthetic construct
  source 1..20: mol_type = other DNA
ccttggagaagtttattatt

SEQ ID NO: 7 - DNA, 20 bp, synthetic construct
  source 1..20: mol_type = other DNA
ggtggtggagcagaaagctt

SEQ ID NO: 8 - DNA, 20 bp, synthetic construct
  source 1..20: mol_type = other DNA
gtgacgataacaagtggttt

SEQ ID NO: 9 - DNA, 20 bp, synthetic construct
  source 1..20: mol_type = other DNA
aacttctccaagggtagctc

SEQ ID NO: 10 - DNA, 20 bp, synthetic construct
  source 1..20: mol_type = other DNA
gttgaagccggagctaccct

SEQ ID NO: 11 - DNA, 20 bp, synthetic construct
  source 1..20: mol_type = other DNA
cccttggagaagtttattat

SEQ ID NO: 12 - DNA, 20 bp, synthetic construct
  source 1..20: mol_type = other DNA
ccaataataaacttctccaa

SEQ ID NO: 13 - DNA, 20 bp, synthetic construct
  source 1..20: mol_type = other DNA
aattaggcgtgaagtagttg

SEQ ID NO: 14 - DNA, 20 bp, synthetic construct
  source 1..20: mol_type = other DNA
tgaaggtggtcacttattgc

SEQ ID NO: 15 - DNA, 20 bp, synthetic construct
  source 1..20: mol_type = other DNA
acttattgctggaagaattc

SEQ ID NO: 16 - DNA, 20 bp, synthetic construct
  source 1..20: mol_type = other DNA
cttattgctggaagaattcc

SEQ ID NO: 17 - DNA, 20 bp, synthetic construct
  source 1..20: mol_type = other DNA
atgaagaagcccttattact

SEQ ID NO: 18 - DNA, 20 bp, synthetic construct
  source 1..20: mol_type = other DNA
attaacggaagaataagaag

SEQ ID NO: 19 - DNA, 20 bp, synthetic construct
  source 1..20: mol_type = other DNA
tattcttccgttaattcaac

SEQ ID NO: 20 - DNA, 20 bp, synthetic construct
  source 1..20: mol_type = other DNA
taggatgtttaatcttgtgg

SEQ ID NO: 21 - DNA, 20 bp, synthetic construct
  source 1..20: mol_type = other DNA
acaccaccccaattcgtcgc

SEQ ID NO: 22 - DNA, 20 bp, synthetic construct
  source 1..20: mol_type = other DNA
tatagaatgttttcactcgt

SEQ ID NO: 23 - DNA, 20 bp, synthetic construct
  source 1..20: mol_type = other DNA
tgggttcttcattatttcct

SEQ ID NO: 24 - DNA, 20 bp, synthetic construct
  source 1..20: mol_type = other DNA
tttagaatgtacaagctcgt

SEQ ID NO: 25 - DNA, 20 bp, synthetic construct
  source 1..20: mol_type = other DNA
aaggtccaagaatgttcgag

SEQ ID NO: 26 - DNA, 20 bp, synthetic construct
  source 1..20: mol_type = other DNA
ggaagggttatcgaaccaaa

SEQ ID NO: 27 - DNA, 20 bp, synthetic construct
  source 1..20: mol_type = other DNA
ggtgaggttgatggagtaag

SEQ ID NO: 28 - DNA, 23 bp, synthetic construct
  source 1..23: mol_type = other DNA
ttgcattattgggaatatattgc

SEQ ID NO: 29 - DNA, 23 bp, synthetic construct
  source 1..23: mol_type = other DNA
attgaacgcatgtttctcaagtc

SEQ ID NO: 30 - DNA, 23 bp, synthetic construct
  source 1..23: mol_type = other DNA
tcattaacccaataataaacttc

SEQ ID NO: 31 - DNA, 23 bp, synthetic construct
  source 1..23: mol_type = other DNA
ctggattgttaggaatatattcc

SEQ ID NO: 32 - DNA, 23 bp, synthetic construct
  source 1..23: mol_type = other DNA
tcctacatatctcaagtcccatt

SEQ ID NO: 33 - DNA, 23 bp, synthetic construct
  source 1..23: mol_type = other DNA
accgtatgcatgtttctcaagtc

SEQ ID NO: 34 - DNA, 23 bp, synthetic construct
  source 1..23: mol_type = other DNA
tcatacttgtaagcaatattttg

SEQ ID NO: 35 - DNA, 23 bp, synthetic construct
  source 1..23: mol_type = other DNA
cttggtggagtggatagtctagt

SEQ ID NO: 36 - DNA, 23 bp, synthetic construct
  source 1..23: mol_type = other DNA
ctgagttgggtattaaaaaaacg

SEQ ID NO: 37 - DNA, 23 bp, synthetic construct
  source 1..23: mol_type = other DNA
tccaattagattgaacattctga

SEQ ID NO: 38 - DNA, 23 bp, synthetic construct
  source 1..23: mol_type = other DNA
ctctgctgttcttcccggaattc

SEQ ID NO: 39 - DNA, 23 bp, synthetic construct
  source 1..23: mol_type = other DNA
atacccaatacttctcaatttcc

SEQ ID NO: 40 - DNA, 23 bp, synthetic construct
  source 1..23: mol_type = other DNA
agcttgatttttcagaagatgaa

SEQ ID NO: 41 - DNA, 23 bp, synthetic construct
  source 1..23: mol_type = other DNA
agaagatgaagaagcccttatta

SEQ ID NO: 42 - DNA, 23 bp, synthetic construct
  source 1..23: mol_type = other DNA
ccccacaagattaaacatcctag

SEQ ID NO: 43 - DNA, 10853 bp, synthetic construct
  source 1..10853: mol_type = other DNA
gttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcctggcgaaagggggatgtgctgcaaggcgattaagttgggtaacgccagggttttcccagtcacgacgttgtaaaacgacggccagtgccaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatacatatgcccgtcgattagcaagttactaccgggtcgacaattctgaatcaacaactctcctggcgcaccatcgtcggctacagcctcgggaattgctaccgagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattggtacctgcggccgctggatcccgggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctggaattctagtatactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaatttgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccacaagctagctttctgagccgccgattttcctcctcgagttggatgaactcgccgagttcatcgtcaactgaaacagacacggccggattctgtgagacaggttgaaccgcagctctcttccattgataataggtctgaacggaaatacccacgatcttaacggcgtccttcaaggttgcgccgccagcgacctgagcttcgatttgaccgatcttctccagtttttctcggttgctgaggccgcgggttttcggcttcacggatttgaacgatcccgtgcgggctgtttcggctggtgctttctttgctcttctacctctaggagcagccggctcaacttcggcagcagcagtaccgtccggcggattctggatctcttcgtcagccattaatcgtcctctgtgtgggttattgctttgtctgccagctcgatccaagagtcaacgtttgtgcctagggcagtaaataggcagtgctccgcgactacatgcctcggccggcaaaataccgccgcatgtagagcaggctctccttcacgatcaacgatcggcatggggccttcgtgcttgttgagtaatgttatcgctcccatcagagcacgcttggtactccgggaatcggatggtctgtcgatcatccaaaaaacgctcatgttttcaacctattaggtctgtggtcagctgaccacagaccatcctgctccatactcgctaattctagccaaaccgcaacgtcccctgcccgctagccttcaagagcgccattatcatcgggccaagtgaaaacttcccgagttcgctccgccgtgtcagatctcggagatagcccccaggcgaattgatgaagttcgctcgctccaaaatgcacgccatcgctgctgccgcattctccggtcccattgcctcacacgcgtcttggtaagccgacgggctgacccccagcatagaccgaaccaccaccgcagccgacatgaggtcacgccagctagcaaccgcaccgctcggcccataattgccaatggtcgggcacgctttcaggatcatcccgagggggaacgcttttatcggctcgctccttgcccggtctatttcactcggcttagcgccctgctccttttcagagcgaggttcaagttcattaacggattcgggttttgagttctgtatgtgctgctcgctctgggcagcattggtgctattattttctgaattgtctctaatttccaaccggttgattatctcttcctggagcatccacatctcttcgagaattgactctacatcagcaagcgtcggggcgcgtgggattctacccacaagttccacatagacttcctcgacagcttgccagtcgccctccgctccctcttccatagctgccgtaattagcttccgaacgtcccgtcggcaaatcgtcagactttctttggccatcctgaatgctgctcgatcggccatcacctgctgtgccatcatcgctagctcttcggaccgcgcgagaagcggagacaaatcgaagccaaacgcgcgctcgatctgaccagcgccatccttacgagcgtaacgctttccgttggcgctatccttccggacgatcaagcctgactccacgagcatggcgatgtgcctacgcaaagtcgcgccagccatcccatgcgcccgaagggcaagctgagcattcgacgggaagacgatcagctgtgcctcctgacgcaactccgtttccgggtgaaagctcaatagcgcatcaaggacggcaagactgttggactggattccaagtagttccatggccgcggacgcgtccctaaagaccttccacttgtccgctgtcttgccttgtttgatatcggccagcgccgtctggcgccgcacaagcgcaagcgtcattggccgccgcccgaatggcgtcgttacacttcctgtctgcatcatctttcacctttcagcaggcaaaggaaatcagctcaccaaaacggcgctaaaaactcttgacgaggattcgaggaaatgcgattctgttcgcgctagagagacagaagggcttccgcgacggcgacgttgagggggctcttttcttttgcggtttactctccccgtttccgttggttctcagcgtggtacgcttgatacagcgctggcacatgatcgagcacgaaggtcgcaaaatcgggcgtcgccttcctgtcaatcgtgatttccagtttggccttgctctgcgtcacctgtgcaattctggtgccgtctggggtggccatgacctcgggaagtccacgcgcaacccgactgggcttcagactagcgatcaccgccttgaatcgttctgccgatggcagcgcttgaacttcctccgacatagcatatttagccacgtcggccggtgaagaaactttctcaatcagctcggcaagttgttgccaactcggccgtccaacaccaggagcggcaccaatagcatcggtcagttcagaggggagggcgtcaacgagcagaagcatcttggacaaattgctcttgtcgatcgacatcgcggcgatgacaatctctcgagaaaactgcctgttcaggcgatgtgcgaagcgcgccttttcgatgaaggtaagatcttcgcgcacattgttttcctgaccctgtgctacgaccacttgctcgtccgtcagttcgcgaacgaccgccctgaccggaagtccgagttctgaaacggcgcgtagccggcggtggccgaaggcaacctgatatcggcccggctggctcggatgcggtcgcacaaggattgggacttgctgtccttgttcccggatcgaagtaaggagcccgtcaatgtcccctcgcatacgatcctgcacgaaagacggttctattgacgaggcatccaactctatcactgcctgaccttcagcgagacgccgctcgatctcttcggcacggctaagacgatcgttttgctctcgcagtgcgttaccaatgttcgctgtgagcttcgttgccggatcgcgctccttccttgttacgccgaggagcggcatggagcggttctttgccgtcctattgtcggcgggcgacgtctcaggggcgtcagttgagacgccaaggatgtgcttccggctcatgtgggcctaccccatgcttttttgatcagtgtttcgatctcgtcgttgacggcgttcatcgcctccaaggctcgatcataggtcgagcgcgtgaacaggccacgctccacttcgaatagagtctggtttgtcaggccagcgtccgaaaccgcggtggttttaagcatcggaaaattgaggacattttcgccaaaaatcgaccgcagataacctaccatttggttctgtggtccgtcgctcggttcgaaacgggttatcagatagcgcatccaattaaacttgaacttggcgccagcattctcgatttcacgcaaaaggttcgatgtcattgccagaaactggttcatcgacatcacatccagcatctgcggatggaccgtgacaagaatggacgtcgccgcagtcaatgcggatagcgtgagatacccaagctggggagggcagtcgatgaccacgacgtcatagttatccgcgatatcttcaattacttggctgatgcgaccataaaagagcgtgtcgccctctttgcggttcatcagcgcgcgtggcgtatcgtgttcaaactccatcagctcaaggttaccaggaatcaggtggaggtcgggaatgtaagtccctcggacgactcgttcgattgccacctgctcatcatcataccttatagcgccgtagagcgtttcgttcgggccaacgtccgtctccggttggctcccaaagagtgcagaaaggctcgcttgaggatcgagatcaatggccaagactcgatatccgcgcatagcgaggtactgcgccagatgcgcggcggtggtggtcttacccgacccacctttgaaattcatcacagagataacctgaagctgctcgccgcctcgacgatgtggcaggtagcgccggttcccgcggccgacctgatccatatacttccgaatcacatggatatcttcaattgagaacattcgcctgccacctgggctcatgctaacattcaactctggcatctcagacgcggtctgccgtaaatatgactcgccaacgccgagcagcttggacgcctccgatggcccgaatgttcgaatacccttctcggaatgcggcgggaaaaccttaagatgatgtgcttgaagttggctcgagagggcatcggcatgacgctccatcaaggccgtcaaccctacaactacaggcgctgcttttaggacagacttcgccatctcaaacccattccttgccagtggcgatatttttcgcgaaactggaaaagttccgccgctggcaattagcgccgattctgctgtttgggcaagagcttttaggttaacagaaggttaacgccctcaggtcgaaaaactccacccaactgttatttgtatttatttccaatgccttagagagattgccatttgaatatgttcatgtattgttttagtgataatcctacaatcgtaacccaaaaagaggtcgccctctgcgcgccgtcgtccaatataggcgaagtcacccttgcgactcaggcggattctaccttgtaggatcgagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctca

SEQ ID NO: 44 - DNA, 1471 bp, synthetic construct
  source 1..1471: mol_type = other DNA
caacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctggaattctagtatactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 45 - DNA, 15488 bp, synthetic construct
  source 1..15488: mol_type = other DNA
gttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcctggcgaaagggggatgtgctgcaaggcgattaagttgggtaacgccagggttttcccagtcacgacgttgtaaaacgacggccagtgccaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatacatatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgagtcgacaattctgaatcaacaactctcctggcgcaccatcgtcggctacagcctcgggaattgctaccgagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattggtacctgcggctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgnnnnnnnnnnnnnnnnnnnngttttagagctagaaatagcaagttaaaataaggctagtccgttatcaacttgaaaaagtggcaccgagtcggtgctttttttctagacccagctttcttgtacaaagttggcattacgctggatcccgggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctggaattctagtatactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaatttgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccacaagctagctttctgagccgccgattttcctcctcgagttggatgaactcgccgagttcatcgtcaactgaaacagacacggccggattctgtgagacaggttgaaccgcagctctcttccattgataataggtctgaacggaaatacccacgatcttaacggcgtccttcaaggttgcgccgccagcgacctgagcttcgatttgaccgatcttctccagtttttctcggttgctgaggccgcgggttttcggcttcacggatttgaacgatcccgtgcgggctgtttcggctggtgctttctttgctcttctacctctaggagcagccggctcaacttcggcagcagcagtaccgtccggcggattctggatctcttcgtcagccattaatcgtcctctgtgtgggttattgctttgtctgccagctcgatccaagagtcaacgtttgtgcctagggcagtaaataggcagtgctccgcgactacatgcctcggccggcaaaataccgccgcatgtagagcaggctctccttcacgatcaacgatcggcatggggccttcgtgcttgttgagtaatgttatcgctcccatcagagcacgcttggtactccgggaatcggatggtctgtcgatcatccaaaaaacgctcatgttttcaacctattaggtctgtggtcagctgaccacagaccatcctgctccatactcgctaattctagccaaaccgcaacgtcccctgcccgctagccttcaagagcgccattatcatcgggccaagtgaaaacttcccgagttcgctccgccgtgtcagatctcggagatagcccccaggcgaattgatgaagttcgctcgctccaaaatgcacgccatcgctgctgccgcattctccggtcccattgcctcacacgcgtcttggtaagccgacgggctgacccccagcatagaccgaaccaccaccgcagccgacatgaggtcacgccagctagcaaccgcaccgctcggcccataattgccaatggtcgggcacgctttcaggatcatcccgagggggaacgcttttatcggctcgctccttgcccggtctatttcactcggcttagcgccctgctccttttcagagcgaggttcaagttcattaacggattcgggttttgagttctgtatgtgctgctcgctctgggcagcattggtgctattattttctgaattgtctctaatttccaaccggttgattatctcttcctggagcatccacatctcttcgagaattgactctacatcagcaagcgtcggggcgcgtgggattctacccacaagttccacatagacttcctcgacagcttgccagtcgccctccgctccctcttccatagctgccgtaattagcttccgaacgtcccgtcggcaaatcgtcagactttctttggccatcctgaatgctgctcgatcggccatcacctgctgtgccatcatcgctagctcttcggaccgcgcgagaagcggagacaaatcgaagccaaacgcgcgctcgatctgaccagcgccatccttacgagcgtaacgctttccgttggcgctatccttccggacgatcaagcctgactccacgagcatggcgatgtgcctacgcaaagtcgcgccagccatcccatgcgcccgaagggcaagctgagcattcgacgggaagacgatcagctgtgcctcctgacgcaactccgtttccgggtgaaagctcaatagcgcatcaaggacggcaagactgttggactggattccaagtagttccatggccgcggacgcgtccctaaagaccttccacttgtccgctgtcttgccttgtttgatatcggccagcgccgtctggcgccgcacaagcgcaagcgtcattggccgccgcccgaatggcgtcgttacacttcctgtctgcatcatctttcacctttcagcaggcaaaggaaatcagctcaccaaaacggcgctaaaaactcttgacgaggattcgaggaaatgcgattctgttcgcgctagagagacagaagggcttccgcgacggcgacgttgagggggctcttttcttttgcggtttactctccccgtttccgttggttctcagcgtggtacgcttgatacagcgctggcacatgatcgagcacgaaggtcgcaaaatcgggcgtcgccttcctgtcaatcgtgatttccagtttggccttgctctgcgtcacctgtgcaattctggtgccgtctggggtggccatgacctcgggaagtccacgcgcaacccgactgggcttcagactagcgatcaccgccttgaatcgttctgccgatggcagcgcttgaacttcctccgacatagcatatttagccacgtcggccggtgaagaaactttctcaatcagctcggcaagttgttgccaactcggccgtccaacaccaggagcggcaccaatagcatcggtcagttcagaggggagggcgtcaacgagcagaagcatcttggacaaattgctcttgtcgatcgacatcgcggcgatgacaatctctcgagaaaactgcctgttcaggcgatgtgcgaagcgcgccttttcgatgaaggtaagatcttcgcgcacattgttttcctgaccctgtgctacgaccacttgctcgtccgtcagttcgcgaacgaccgccctgaccggaagtccgagttctgaaacggcgcgtagccggcggtggccgaaggcaacctgatatcggcccggctggctcggatgcggtcgcacaaggattgggacttgctgtccttgttcccggatcgaagtaaggagcccgtcaatgtcccctcgcatacgatcctgcacgaaagacggttctattgacgaggcatccaactctatcactgcctgaccttcagcgagacgccgctcgatctcttcggcacggctaagacgatcgttttgctctcgcagtgcgttaccaatgttcgctgtgagcttcgttgccggatcgcgctccttccttgttacgccgaggagcggcatggagcggttctttgccgtcctattgtcggcgggcgacgtctcaggggcgtcagttgagacgccaaggatgtgcttccggctcatgtgggcctaccccatgcttttttgatcagtgtttcgatctcgtcgttgacggcgttcatcgcctccaaggctcgatcataggtcgagcgcgtgaacaggccacgctccacttcgaatagagtctggtttgtcaggccagcgtccgaaaccgcggtggttttaagcatcggaaaattgaggacattttcgccaaaaatcgaccgcagataacctaccatttggttctgtggtccgtcgctcggttcgaaacgggttatcagatagcgcatccaattaaacttgaacttggcgccagcattctcgatttcacgcaaaaggttcgatgtcattgccagaaactggttcatcgacatcacatccagcatctgcggatggaccgtgacaagaatggacgtcgccgcagtcaatgcggatagcgtgagatacccaagctggggagggcagtcgatgaccacgacgtcatagttatccgcgatatcttcaattacttggctgatgcgaccataaaagagcgtgtcgccctctttgcggttcatcagcgcgcgtggcgtatcgtgttcaaactccatcagctcaaggttaccaggaatcaggtggaggtcgggaatgtaagtccctcggacgactcgttcgattgccacctgctcatcatcataccttatagcgccgtagagcgtttcgttcgggccaacgtccgtctccggttggctcccaaagagtgcagaaaggctcgcttgaggatcgagatcaatggccaagactcgatatccgcgcatagcgaggtactgcgccagatgcgcggcggtggtggtcttacccgacccacctttgaaattcatcacagagataacctgaagctgctcgccgcctcgacgatgtggcaggtagcgccggttcccgcggccgacctgatccatatacttccgaatcacatggatatcttcaattgagaacattcgcctgccacctgggctcatgctaacattcaactctggcatctcagacgcggtctgccgtaaatatgactcgccaacgccgagcagcttggacgcctccgatggcccgaatgttcgaatacccttctcggaatgcggcgggaaaaccttaagatgatgtgcttgaagttggctcgagagggcatcggcatgacgctccatcaaggccgtcaaccctacaactacaggcgctgcttttaggacagacttcgccatctcaaacccattccttgccagtggcgatatttttcgcgaaactggaaaagttccgccgctggcaattagcgccgattctgctgtttgggcaagagcttttaggttaacagaaggttaacgccctcaggtcgaaaaactccacccaactgttatttgtatttatttccaatgccttagagagattgccatttgaatatgttcatgtattgttttagtgataatcctacaatcgtaacccaaaaagaggtcgccctctgcgcgccgtcgtccaatataggcgaagtcacccttgcgactcaggcggattctaccttgtaggatcgagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctca

SEQ ID NO: 46 - DNA, 4140 bp, synthetic construct
  source 1..4140: mol_type = other DNA
atggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttga

SEQ ID NO: 47 - DNA, 525 bp, synthetic construct
  source 1..525: mol_type = other DNA
ctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgnnnnnnnnnnnnnnnnnnnngttttagagctagaaatagcaagttaaaataaggctagtccgttatcaacttgaaaaagtggcaccgagtcggtgctttttttctagacccagctttcttgtacaaagttggcatta

SEQ ID NO: 48 - DNA, 10339 bp, synthetic construct
  source 1..10339: mol_type = other DNA
aagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatacatatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgagtcgacaattctgaatcaacaactctcctggcgcaccatcgtcggctacagcctcgggaattgctaccgagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattggtacctgcggctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgnnnnnnnnnnnnnnnnnnnngttttagagctagaaatagcaagttaaaataaggctagtccgttatcaacttgaaaaagtggcaccgagtcggtgctttttttctagacccagctttcttgtacaaagttggcattacgctggatcccgggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctggaattctagtatactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaa

SEQ ID NO: 49 - DNA, 5704 bp, synthetic construct
  source 1..5704: mol_type = other DNA
aagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatacatatgcccgtcgattagcaagttactaccgggtcgacaattctgaatcaacaactctcctggcgcaccatcgtcggctacagcctcgggaattgctaccgagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattggtacctgcggccgctggatcccgggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctggaattctagtatactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaa

SEQ ID NO: 50 - DNA, 3697 bp, synthetic construct
  source 1..3697: mol_type = other DNA
aagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatacatatgcccgtcgattagcaagttactaccgggtcgacaattctgaatcaacaactctcctggcgcaccatcgtcggctacagcctcgggaattgctaccgagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattggtacctgcggccgctggatcccgggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaa

SEQ ID NO: 51 - DNA, 6395 bp, synthetic construct
  source 1..6395: mol_type = other DNA
aagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatacatatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaattctgaatcaacaactctcctggcgcaccatcgtcggctacagcctcgggaattgctaccgagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattggtacctgcggccgctggatcccgggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctggaattctagtatactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaa

SEQ ID NO: 52 - DNA, 4388 bp, synthetic construct
  source 1..4388: mol_type = other DNA
aagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatacatatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaattctgaatcaacaactctcctggcgcaccatcgtcggctacagcctcgggaattgctaccgagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattggtacctgcggccgctggatcccgggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaa

SEQ ID NO: 53 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 54 - DNA, 7466 bp, synthetic construct
  source 1..7466: mol_type = other DNA
aagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatacatatggcaaccggagaaaacagaacggtgccggacaatctaaagaaacagctcgcagtttcagttcgaaacattcaatggagttatggaatcttctggtctgtctctgcttctcaaccaggagtgttggagtggggagatggatattacaatggagacataaagacaaggaagacgattcaagcagcagaagtcaaaattgaccagttaggtcttgagagaagtgagcagcttagagagctttatgaatctctctccctcgctgaatcctcagcttccggtagctctcaggtcactagacgagcttccgccgccgctctctcaccggaggacctcaccgacaccgagtggtactacttagtatgcatgtctttcgtcttcaacatcggtgaaggaatccccggaggagcgttatccaatggagaaccaatatggctttgtaacgctgaaaccgccgatagcaaagtcttcactcgttctcttctagctaaaagtgcttcgcttcagacagtggtttgcttcccgtttcttggaggagtccttgagattggcacgaccgaacatattaaagaggacatgaacgtgatacaaagtgttaagacgttgttccttgaagctcctccatatactacaatatcgacaagatcagactatcaagaaatttttgatcccttaagtgacgataaatacactccggtgtttataaccgaagcttttccaacaacttctactagcgggtttgagcaagaacctgaggatcatgattcgttcatcaacgatggtggtgcgtctcaggtacaaagctggcagtttgtgggtgaagaaatcagtaactgcattcaccaatcgttaaattcaagcgattgcgtttcccaaacgtttgttggaacaaccgggagacttgcttgcgatccaaggaagagtaggattcaacggttaggtcagattcaagaacagagtaaccatgtaaatatggacgacgatgttcattaccaaggcgtgatatcgacgattttcaaaacaacgcatcagctaatactcggaccgcagtttcagaacttcgataagcggtctagcttcacaaggtggaagcgatcatcatctgtgaaaacattgggagagaaatcgcagaagatgataaagaagatactcttcgaggttcctttgatgaacaagaaagaagagttgttaccggacacaccagaggaaaccgggaaccatgccttgtccgagaagaaacgccgcgagaaattgaatgaacggtttatgacattgagatcaatcattccctcaattagtaagattgataaagtgtcgattcttgatgatacaattgagtatcttcaagatttacagaaacgggttcaagagttggaatcttgtagagaatctgctgatacagagacacggataacgatgatgaagaggaagaaaccggatgatgaggaggaaagagcatcagcgaattgtatgaacagcaaaaggaaggggagtgatgtgaatgtaggagaagatgaaccagctgatatcggttatgctggtctaacggataacttaaggatcagttcattaggtaacgaggtggttattgagcttagatgcgcttggagagaagggatattgcttgagataatggatgtgattagtgatctcaacttggattctcactcggttcagtcgtcaaccggagacggtttactctgcttaactgtcaattgcaagcataaagggacaaaaatagcaacaacaggaatgatccaagaggcacttcaaagggttgcatggatatgttaagtcgacaattctgaatcaacaactctcctggcgcaccatcgtcggctacagcctcgggaattgctaccgagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattggtacctgcggccgctggatcccgggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctggaattctagtatactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaa

SEQ ID NO: 55 - DNA, 1791 bp, synthetic construct
  source 1..1791: mol_type = other DNA
atggcaaccggagaaaacagaacggtgccggacaatctaaagaaacagctcgcagtttcagttcgaaacattcaatggagttatggaatcttctggtctgtctctgcttctcaaccaggagtgttggagtggggagatggatattacaatggagacataaagacaaggaagacgattcaagcagcagaagtcaaaattgaccagttaggtcttgagagaagtgagcagcttagagagctttatgaatctctctccctcgctgaatcctcagcttccggtagctctcaggtcactagacgagcttccgccgccgctctctcaccggaggacctcaccgacaccgagtggtactacttagtatgcatgtctttcgtcttcaacatcggtgaaggaatccccggaggagcgttatccaatggagaaccaatatggctttgtaacgctgaaaccgccgatagcaaagtcttcactcgttctcttctagctaaaagtgcttcgcttcagacagtggtttgcttcccgtttcttggaggagtccttgagattggcacgaccgaacatattaaagaggacatgaacgtgatacaaagtgttaagacgttgttccttgaagctcctccatatactacaatatcgacaagatcagactatcaagaaatttttgatcccttaagtgacgataaatacactccggtgtttataaccgaagcttttccaacaacttctactagcgggtttgagcaagaacctgaggatcatgattcgttcatcaacgatggtggtgcgtctcaggtacaaagctggcagtttgtgggtgaagaaatcagtaactgcattcaccaatcgttaaattcaagcgattgcgtttcccaaacgtttgttggaacaaccgggagacttgcttgcgatccaaggaagagtaggattcaacggttaggtcagattcaagaacagagtaaccatgtaaatatggacgacgatgttcattaccaaggcgtgatatcgacgattttcaaaacaacgcatcagctaatactcggaccgcagtttcagaacttcgataagcggtctagcttcacaaggtggaagcgatcatcatctgtgaaaacattgggagagaaatcgcagaagatgataaagaagatactcttcgaggttcctttgatgaacaagaaagaagagttgttaccggacacaccagaggaaaccgggaaccatgccttgtccgagaagaaacgccgcgagaaattgaatgaacggtttatgacattgagatcaatcattccctcaattagtaagattgataaagtgtcgattcttgatgatacaattgagtatcttcaagatttacagaaacgggttcaagagttggaatcttgtagagaatctgctgatacagagacacggataacgatgatgaagaggaagaaaccggatgatgaggaggaaagagcatcagcgaattgtatgaacagcaaaaggaaggggagtgatgtgaatgtaggagaagatgaaccagctgatatcggttatgctggtctaacggataacttaaggatcagttcattaggtaacgaggtggttattgagcttagatgcgcttggagagaagggatattgcttgagataatggatgtgattagtgatctcaacttggattctcactcggttcagtcgtcaaccggagacggtttactctgcttaactgtcaattgcaagcataaagggacaaaaatagcaacaacaggaatgatccaagaggcacttcaaagggttgcatggatatgttaa

SEQ ID NO: 56 - DNA, 9997 bp, synthetic construct
  source 1..9997: mol_type = other DNA
aagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatacatatggctcctaagaagaagcggaaggttggtattcacggggtgcctgcggcttcaaagctcgagaaattcaccaactgttattcgttgagcaaaacactgcggtttaaagcgattccagtcggcaagactcaagagaatatagacaataagcggctgttggtggaagatgaaaagcgcgcggaagactacaaaggggtgaagaagttgttggacagatactacctctcttttatcaatgatgtcttgcactcaatcaaattgaagaatctgaacaactacatctccctcttcagaaagaaaacaaggacagaaaaggagaataaggaacttgaaaatttggagatcaatctgaggaaagagatcgcgaaagcctttaaaggcaacgaaggatacaaaagtctgttcaagaaggatataattgagacaattttgccagagttcctcgatgacaaggacgagattgcgctggtcaattcgttcaacggattcacaacagcattcacaggcttctttgataatcgggaaaatatgttctctgaggaggcaaagtccacttctattgcgttcaggtgtatcaatgagaatctcactaggtacatttccaacatggatatctttgagaaggttgacgcaatttttgacaagcacgaagttcaggagattaaggagaagatcctcaattccgattatgacgttgaggacttcttcgaaggtgagttttttaatttcgtgctcactcaagagggtatcgacgtgtataatgcgatcatcggtgggttcgtgactgagtccggtgaaaagattaagggattgaacgagtatatcaacctttacaaccaaaagacgaaacagaagctgccaaagttcaagcctctttacaaacaggttctttcagaccgcgagtcactctcgttctatggggagggctacacttcggatgaggaagtcctggaggtgttcaggaatactctcaataagaattcggagattttctcttctataaaaaaactggaaaagttgtttaagaattttgacgaatactctagcgccggcatatttgtgaaaaacggcccggccatatcaacgataagtaaagatatcttcggcgaatggaacgtgatcagagacaaatggaacgcggagtatgacgatattcacctgaagaagaaggctgtcgtaacggagaagtacgaggatgatcgcaggaaaagcttcaaaaagatcggaagtttcagcctggaacagttgcaggagtatgctgacgccgatcttagcgtcgtcgagaagttgaaggagataatcatccaaaaggtcgacgagatatataaagtctatggatcaagtgaaaaactgttcgacgccgacttcgttttggagaagtccctgaagaagaacgacgctgttgttgccattatgaaggatctgctcgacagcgtgaagagtttcgagaactatattaaggcttttttcggggaggggaaggagactaacagagatgagtccttctacggagacttcgtcctcgcgtacgatatactccttaaggtagaccacatctacgacgcaatcagaaattacgtgacacaaaagccgtacagcaaggacaagttcaaactctacttccagaacccccagttcatgggcggctgggacaaggacaaggaaacggattacagggctacgatcctgaggtatggttcaaaatactacttggcgattatggacaagaagtacgccaagtgtctccagaagattgacaaagacgatgtcaatggcaattatgagaagatcaactacaagctgcttccgggtccgaacaagatgctcccaaaggttttcttcagcaagaaatggatggcctactataacccaagcgaggacatccagaagatttataagaacggtacgttcaagaagggcgacatgttcaatcttaacgactgtcacaagctgatcgacttcttcaaagactcaattagccggtacccaaagtggtctaacgcctatgacttcaacttttcggaaaccgagaagtacaaggatatagccggattttatagagaggtggaagagcagggctacaaggtgtcattcgagtccgccagcaagaaggaagtggacaagctcgtggaagagggtaagctctacatgttccagatttataataaagactttagcgataagagccacgggacacctaatctccacacaatgtatttcaagctgctcttcgacgagaataaccacggccaaatcaggttgtcaggaggggctgaactcttcatgcggcgcgctagccttaagaaggaggagcttgtagtccaccctgcgaatagtccaattgcgaataagaacccggacaatcctaaaaagactacaacattgagctacgacgtgtacaaggataagaggttttccgaggatcagtacgagctccacatcccgattgcgatcaacaagtgcccaaagaatattttcaagataaacacagaggtgcgtgtactcctgaagcatgacgacaatccttacgtcattgggattgatcggggcgagaggaacctcctctatattgtggtggtggacgggaaggggaacatagtcgaacagtactcccttaacgaaataattaacaatttcaacggcatccgtatcaagaccgactaccattcgttgctggacaagaaggagaaggagagatttgaggcgcggcaaaattggacaagtatcgagaacatcaaggaactcaaagcaggttatatctctcaagttgtgcataagatatgcgagctggttgagaagtatgacgcagtgatcgctcttgaggacctcaactcgggctttaagaattctagagttaaagtggagaagcaggtctatcaaaagttcgagaagatgcttatagataagctcaactacatggtcgataagaaatcgaacccatgtgccaccggcggcgcactcaaaggttaccaaataacaaacaaattcgagtccttcaaatcgatgagtactcagaatgggttcatattttatataccggcgtggcttacgtctaagatcgacccgtcaactggttttgtcaacctgttgaagacgaaatacacgtccattgccgattcgaaaaagttcatatctagttttgatcgtattatgtacgtcccagaggaagatcttttcgagtttgctctcgactacaaaaacttttcgcggaccgatgcggattacattaaaaaatggaaactctattcgtacggcaacagaatcaggatttttcgcaaccctaagaagaataacgtctttgattgggaggaagtttgcttgactagcgcgtacaaggagctctttaataagtatggcattaactaccaacagggtgatatcagagcactgctttgcgaacaatctgacaaggctttctactcatccttcatggctttgatgagcctgatgctccagatgagaaattcaattacaggcagaaccgacgtggatttcttgatctccccggttaaaaattctgatggcatcttttacgatagcaggaactatgaagcgcaagagaatgcgattctgccaaaaaatgcagacgccaacggtgcctataacatcgccaggaaagtcctgtgggcgatcggccagttcaaaaaggccgaagacgaaaaattggacaaggtcaaaatcgctatcagcaacaaagagtggctggagtatgctcagacatccgtaaagcataagcgtcctgctgccaccaaaaaggccggacaggctaagaaaaagaagtgagtcgacaattctgaatcaacaactctcctggcgcaccatcgtcggctacagcctcgggaattgctaccgagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattggtacctgcggctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtaatttctactaagtgtagatnnnnnnnnnnnnnnnnnnnnnggccggcatggtcccagcctcctcgctggcgccggctgggcaacatgcttcggcatggcgaatgggacttttttttctagacccagctttcttgtacaaagttggcattacgctggatcccgggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctggaattctagtatactaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaa

SEQ ID NO: 57 - DNA, 3783 bp, synthetic construct
  source 1..3783: mol_type = other DNA
atggctcctaagaagaagcggaaggttggtattcacggggtgcctgcggcttcaaagctcgagaaattcaccaactgttattcgttgagcaaaacactgcggtttaaagcgattccagtcggcaagactcaagagaatatagacaataagcggctgttggtggaagatgaaaagcgcgcggaagactacaaaggggtgaagaagttgttggacagatactacctctcttttatcaatgatgtcttgcactcaatcaaattgaagaatctgaacaactacatctccctcttcagaaagaaaacaaggacagaaaaggagaataaggaacttgaaaatttggagatcaatctgaggaaagagatcgcgaaagcctttaaaggcaacgaaggatacaaaagtctgttcaagaaggatataattgagacaattttgccagagttcctcgatgacaaggacgagattgcgctggtcaattcgttcaacggattcacaacagcattcacaggcttctttgataatcgggaaaatatgttctctgaggaggcaaagtccacttctattgcgttcaggtgtatcaatgagaatctcactaggtacatttccaacatggatatctttgagaaggttgacgcaatttttgacaagcacgaagttcaggagattaaggagaagatcctcaattccgattatgacgttgaggacttcttcgaaggtgagttttttaatttcgtgctcactcaagagggtatcgacgtgtataatgcgatcatcggtgggttcgtgactgagtccggtgaaaagattaagggattgaacgagtatatcaacctttacaaccaaaagacgaaacagaagctgccaaagttcaagcctctttacaaacaggttctttcagaccgcgagtcactctcgttctatggggagggctacacttcggatgaggaagtcctggaggtgttcaggaatactctcaataagaattcggagattttctcttctataaaaaaactggaaaagttgtttaagaattttgacgaatactctagcgccggcatatttgtgaaaaacggcccggccatatcaacgataagtaaagatatcttcggcgaatggaacgtgatcagagacaaatggaacgcggagtatgacgatattcacctgaagaagaaggctgtcgtaacggagaagtacgaggatgatcgcaggaaaagcttcaaaaagatcggaagtttcagcctggaacagttgcaggagtatgctgacgccgatcttagcgtcgtcgagaagttgaaggagataatcatccaaaaggtcgacgagatatataaagtctatggatcaagtgaaaaactgttcgacgccgacttcgttttggagaagtccctgaagaagaacgacgctgttgttgccattatgaaggatctgctcgacagcgtgaagagtttcgagaactatattaaggcttttttcggggaggggaaggagactaacagagatgagtccttctacggagacttcgtcctcgcgtacgatatactccttaaggtagaccacatctacgacgcaatcagaaattacgtgacacaaaagccgtacagcaaggacaagttcaaactctacttccagaacccccagttcatgggcggctgggacaaggacaaggaaacggattacagggctacgatcctgaggtatggttcaaaatactacttggcgattatggacaagaagtacgccaagtgtctccagaagattgacaaagacgatgtcaatggcaattatgagaagatcaactacaagctgcttccgggtccgaacaagatgctcccaaaggttttcttcagcaagaaatggatggcctactataacccaagcgaggacatccagaagatttataagaacggtacgttcaagaagggcgacatgttcaatcttaacgactgtcacaagctgatcgacttcttcaaagactcaattagccggtacccaaagtggtctaacgcctatgacttcaacttttcggaaaccgagaagtacaaggatatagccggattttatagagaggtggaagagcagggctacaaggtgtcattcgagtccgccagcaagaaggaagtggacaagctcgtggaagagggtaagctctacatgttccagatttataataaagactttagcgataagagccacgggacacctaatctccacacaatgtatttcaagctgctcttcgacgagaataaccacggccaaatcaggttgtcaggaggggctgaactcttcatgcggcgcgctagccttaagaaggaggagcttgtagtccaccctgcgaatagtccaattgcgaataagaacccggacaatcctaaaaagactacaacattgagctacgacgtgtacaaggataagaggttttccgaggatcagtacgagctccacatcccgattgcgatcaacaagtgcccaaagaatattttcaagataaacacagaggtgcgtgtactcctgaagcatgacgacaatccttacgtcattgggattgatcggggcgagaggaacctcctctatattgtggtggtggacgggaaggggaacatagtcgaacagtactcccttaacgaaataattaacaatttcaacggcatccgtatcaagaccgactaccattcgttgctggacaagaaggagaaggagagatttgaggcgcggcaaaattggacaagtatcgagaacatcaaggaactcaaagcaggttatatctctcaagttgtgcataagatatgcgagctggttgagaagtatgacgcagtgatcgctcttgaggacctcaactcgggctttaagaattctagagttaaagtggagaagcaggtctatcaaaagttcgagaagatgcttatagataagctcaactacatggtcgataagaaatcgaacccatgtgccaccggcggcgcactcaaaggttaccaaataacaaacaaattcgagtccttcaaatcgatgagtactcagaatgggttcatattttatataccggcgtggcttacgtctaagatcgacccgtcaactggttttgtcaacctgttgaagacgaaatacacgtccattgccgattcgaaaaagttcatatctagttttgatcgtattatgtacgtcccagaggaagatcttttcgagtttgctctcgactacaaaaacttttcgcggaccgatgcggattacattaaaaaatggaaactctattcgtacggcaacagaatcaggatttttcgcaaccctaagaagaataacgtctttgattgggaggaagtttgcttgactagcgcgtacaaggagctctttaataagtatggcattaactaccaacagggtgatatcagagcactgctttgcgaacaatctgacaaggctttctactcatccttcatggctttgatgagcctgatgctccagatgagaaattcaattacaggcagaaccgacgtggatttcttgatctccccggttaaaaattctgatggcatcttttacgatagcaggaactatgaagcgcaagagaatgcgattctgccaaaaaatgcagacgccaacggtgcctataacatcgccaggaaagtcctgtgggcgatcggccagttcaaaaaggccgaagacgaaaaattggacaaggtcaaaatcgctatcagcaacaaagagtggctggagtatgctcagacatccgtaaagcataagcgtcctgctgccaccaaaaaggccggacaggctaagaaaaagaagtga

SEQ ID NO: 58 - DNA, 540 bp, synthetic construct
  source 1..540: mol_type = other DNA
ctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtaatttctactaagtgtagatnnnnnnnnnnnnnnnnnnnnnggccggcatggtcccagcctcctcgctggcgccggctgggcaacatgcttcggcatggcgaatgggacttttttttctagacccagctttcttgtacaaagttggcatta

SEQ ID NO: 59 - RNA, 21 nt, synthetic construct
  source 1..21: mol_type = other RNA
ccctgctccaatatataaagc

SEQ ID NO: 60 - RNA, 19 nt, synthetic construct
  source 1..19: mol_type = other RNA
atacacagtacatccggac

SEQ ID NO: 61 - DNA, 1635 bp, Cannabis sp.
  source 1..1635: mol_type = genomic DNA
atgaagtactcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcagctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagagtcttagtttggctgctgggtattgccctactgtttgcgcaggtggacactttggtggaggaggctatggaccattgatgagaagctatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttgaaaaaattatatgaagaagatataggagctgggatgtatgcgttgtacccttacggtggtataatggatgagatttctgaatcagcaattccattccctcatcgagctggaatcttgtatgagttatggtacatatgtagctgggagaagcaagaagataacgaaaagcatctaaactggattagaaatatttataacttcatgactccttatgtgtcccaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataatttttttagaaacgaacaaagcatcccacctcttccacggcatcatcattaa

SEQ ID NO: 62 - protein, 544 aa, Cannabis sp.
MKYSTFSFWFVCKIIFFFFSFNIQTSIANPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILKKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHHH

SEQ ID NO: 63 - RNA, 46 nt, synthetic construct
  source 1..46: mol_type = other RNA
agcaagttcttcactgttgatacatccctgctccaatatataaagc

SEQ ID NO: 64 - RNA, 44 nt, synthetic construct
  source 1..44: mol_type = other RNA
gagttgttgattcagaattgtcgacatacacagtacatccggac

SEQ ID NO: 65 - DNA, 1636 bp, synthetic construct
  source 1..1636: mol_type = other DNA
atgaagtactcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcagctctgacacaaccccaaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagagtcttagtttggctgctgggtattgccctactgtttgcgcaggtggacactttggtggaggaggctatggaccattgatgagaagctatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttgaaaaaattatatgaagaagatataggagctgggatgtatgcgttgtacccttacggtggtataatggatgagatttctgaatcagcaattccattccctcatcgagctggaatcttgtatgagttatggtacatatgtagctgggagaagcaagaagataacgaaaagcatctaaactggattagaaatatttataacttcatgactccttatgtgtcccaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataatttttttagaaacgaacaaagcatcccacctcttccacggcatcatcattaa

SEQ ID NO: 66 - DNA, 1633 bp, synthetic construct
  source 1..1633: mol_type = other DNA
atgaagtactcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcagctctgacacaaccccaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagagtcttagtttggctgctgggtattgccctactgtttgcgcaggtggacactttggtggaggaggctatggaccattgatgagaagctatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttgaaaaaattatatgaagaagatataggagctgggatgtatgcgttgtacccttacggtggtataatggatgagatttctgaatcagcaattccattccctcatcgagctggaatcttgtatgagttatggtacatatgtagctgggagaagcaagaagataacgaaaagcatctaaactggattagaaatatttataacttcatgactccttatgtgtcccaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataatttttttagaaacgaacaaagcatcccacctcttccacggcatcatcattaa

SEQ ID NO: 67 - DNA, 1631 bp, synthetic construct
  source 1..1631: mol_type = other DNA
atgaagtactcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcagctctgacacaaccccaaaattgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagagtcttagtttggctgctgggtattgccctactgtttgcgcaggtggacactttggtggaggaggctatggaccattgatgagaagctatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttgaaaaaattatatgaagaagatataggagctgggatgtatgcgttgtacccttacggtggtataatggatgagatttctgaatcagcaattccattccctcatcgagctggaatcttgtatgagttatggtacatatgtagctgggagaagcaagaagataacgaaaagcatctaaactggattagaaatatttataacttcatgactccttatgtgtcccaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataatttttttagaaacgaacaaagcatcccacctcttccacggcatcatcattaa

SEQ ID NO: 68 - protein, 114 aa, synthetic construct
MKYSTFSFWFVCKIIFFFFSFNIQTSIANPRENFLKCFSQYIPNNATNLKLVYTQNNPLYMSVLNSTIHNLRFSSDTTPKTTCYRHSFTCLSYPRHYSMLQESWLADSNSKWWS

SEQ ID NO: 69 - protein, 113 aa, synthetic construct
MKYSTFSFWFVCKIIFFFFSFNIQTSIANPRENFLKCFSQYIPNNATNLKLVYTQNNPLYMSVLNSTIHNLRFSSDTTPTTCYRHSFTCLSYPRHYSMLQESWLADSNSKWWS

SEQ ID NO: 70 - protein, 127 aa, synthetic construct
MKYSTFSFWFVCKIIFFFFSFNIQTSIANPRENFLKCFSQYIPNNATNLKLVYTQNNPLYMSVLNSTIHNLRFSSDTTPKLLSSLLHMSLISKALFYAPRKLACRFELEVVVMILRACPTYLKSHLL